(19) United States
(12) Patent Application Publication
(10) Pub. No.: US 2023/0174999 A1
(43) Pub. Date: Jun. 8, 2023

(54) SYSTEMS AND METHODS FOR HIGH YIELDING RECOMBINANT MICROORGANISMS AND USES THEREOF

(71) Applicant: CLARA FOODS CO., South San Francisco, CA (US)

(72) Inventors: Frank Douglas IVEY, South San Francisco, CA (US); Joel Andrew KREPS, South San Francisco, CA (US); Charles Albert TINDELL, South San Francisco, CA (US); Weixi ZHONG, South San Francisco, CA (US)

(21) Appl. No.: 17/816,049

(22) Filed: Jul. 29, 2022

Related U.S. Application Data
(63) Continuation of application No. PCT/US2021/016658, filed on Feb. 4, 2021.
(60) Provisional application No. 62/970,052, filed on Feb. 4, 2020.

(51) Int. Cl. C12N 15/81; C07K 14/465; A23J 1/18

(52) U.S. Cl. CPC C12N 15/815; C07K 14/465; A23J 1/18; C12N 2830/002

(57) ABSTRACT

Provided are systems and methods for high-yield production of recombinant proteins in engineered microorganisms.

### CROSS-REFERENCE TO RELATED APPLICATIONS

This application is a continuation of International Patent Application PCT/US2021/016658, filed Feb. 4, 2021, which claims priority to U.S. Provisional Patent Application Ser. No. 62/970,052, filed Feb. 4, 2020; each of which is incorporated herein by reference in its entirety.

### SEQUENCE LISTING

The instant application contains a Sequence Listing which has been submitted in XML format via Patent Center and is hereby incorporated by reference in its entirety. Said XML copy, created on Jul. 19, 2022, is named 49160-719.301.xml and is 74,302 bytes in size.

### BACKGROUND

In industrial protein production, a goal towards cost reduction is to maximize expression of the protein product in the recombinant organism. Methylotrophic yeasts such as Pichia sp. are an important production system for proteins. Despite their widespread use, high yield expression, particularly for expression of heterologous animal-derived proteins remains a challenge. This hurdle is particularly apparent in larger scale fermentation settings. While increasing the number of integrated copies can lead to increases in protein expression, there appear to be limitations to the amount of transcript produced with increasing copy number (Aw and Polizzi; Microb Cell Fact. 2013; 12: 128).

There is a growing demand for animal-free proteins, particularly in food product-based ingredients. For example, an observable trend of preference for health-conscious fast food options has seen egg white demand at all-time highs in recent years. Aside from an increasingly health conscious consumer base, aversion to the inhumane aspects of the industrial hatchery may fuel acceptance and ultimately preference of animal-free egg white alternatives over factory-farmed eggs. Thus, there is a need for novel methods for high-yield industrial production of food proteins, e.g., alternative animal-free egg proteins.

### SUMMARY

The present invention addresses this need. The systems and methods provide high-titer expression of recombinant proteins in large scale production and are particularly useful for expressing heterologous animal derived proteins in a microbial host, such as food-based proteins.

Accordingly, the present disclosure provides an engineered host cell for expressing a heterologous protein, said engineering host cell may comprise at least three different expression cassettes integrated into the genome of the engineered host cell wherein; a first expression cassette may comprise a first promoter operably linked to a heterologous gene sequence encoding the heterologous protein; a second expression cassette may comprise a second promoter operably linked to a heterologous gene sequence encoding the heterologous protein; a third expression cassette may comprise a third promoter operably linked to a helper factor sequence. In some embodiments, a copy number ratio of the helper factor encoding sequence to the heterologous protein encoding sequence may be at least 1:10.

In some aspects, an engineered host cell for expressing a heterologous protein is provided herein wherein said engineering host cell may comprise at least three different expression cassettes integrated into the genome of the engineered host cell. In some cases, a first expression cassette may comprise a first promoter operably linked to a heterologous gene sequence encoding the heterologous protein; a second expression cassette may comprise a second promoter operably linked to a heterologous gene sequence encoding the heterologous protein; a third expression cassette may comprise a third promoter operably linked to a helper factor sequence; and a copy number ratio of the helper factor encoding sequence to the heterologous protein encoding sequence may be at most 1:2.

In some aspects, provided herein are methods of producing a recombinant heterologous protein in a host cell. The methods may comprise: transforming a plurality of plasmids into the host cell; wherein the plurality of plasmids may comprise at least three plasmids, each one of the at least three plasmids may comprise a different expression cassette; wherein each of the different expression cassettes may comprise a different promoter operably linked to a heterologous gene sequence. The method comprises allowing the integration of at least one copy of each of the at least three different expression cassettes into the host cell. In some cases, at least one of the at least three expression cassettes may comprise a promoter operably linked to a helper factor gene sequence. In some cases, a copy number ratio of the helper factor gene to the heterologous gene to may be at least 1:10.

In some aspects, provided herein are methods of producing a recombinant heterologous protein in a host cell. In some embodiments, the method may comprise: transforming a plurality of plasmids into the host cell; wherein the plurality of plasmids may comprise at least three plasmids, each one of the at least three plasmids may comprise a different expression cassette; wherein each of the different expression cassettes may comprise a different promoter operably linked to a heterologous gene sequence; allowing the integration of at least one copy of each of the at least three different expression cassettes into the host cell; wherein at least one of the at least three expression cassettes may comprise a promoter operably linked to a helper factor gene sequence. In some cases, a copy number ratio of the helper factor gene to the heterologous gene to may be at most 1:2.

In some embodiments, the methods may further comprise identifying the integrated expression cassettes. In some embodiments, the identifying may comprise sequencing the host cell genome. In some embodiments, the identifying may comprise determining the presence or absence of promoters operably linked to the heterologous gene sequence. In some embodiments, the method further may comprise transforming at least one plasmid may comprise one or more expression cassettes, wherein each of the expression cassettes comprise a promoter operably linked to the heterologous gene sequence, wherein the promoter was identified to be present in the host cell genome. In some embodiments, the method may further comprise transforming at least one plasmid may comprise one or more expression cassettes, wherein each of the expression cassettes comprise a promoter operably linked to the heterologous gene sequence, wherein the promoter was identified to be absent in the host cell genome.

In some embodiments, the copy number ratio of the helper factor encoding sequence to the heterologous protein encoding sequence may be at least 1:10, 1:9, 1:8, 1:7, 1:6, 1:5, 1:4 or 1:3. In some embodiments, the copy number ratio of the helper factor encoding sequence to the heterologous protein encoding sequence may be at most 1:9, 1:8, 1:7, 1:6, 1:5, 1:4, 1:3 or 1:2.

In some embodiments, the at least one promoter may be an inducible promoter. In some embodiments, all of the promoters are inducible promoters. In some embodiments, the inducible promoter may be a methanol inducible promoter. In some embodiments, each methanol inducible promoter may be independently selected from the group consisting of AOX1, AOX2, DAK2, DAS2, FDH1, FGH1, FLD1, and PEX11 or a methanol inducible fragment thereof.

In some embodiments, at least one promoter may be a constitutive promoter. In some embodiments, the constitutive promoters may be independently selected from the group consisting of GAP and GCW14.

In some embodiments, the host cell may comprise at least 2 copies of the first expression cassette. In some embodiments, the host cell may comprise at least 2 copies of the second expression cassette. In some embodiments, the host cell may comprise at least 1 copy of a fourth expression cassette may comprise a fourth promoter operably linked to the heterologous gene sequence. In some embodiments, the first cassette and the second cassette are integrated into the genome in the same 5′ to 3′ orientation. In some embodiments, the first cassette and the second cassette are integrated into the genome in an opposite 5′ to 3′ orientation.

In some embodiments, the host cell may comprise at least 2 copies of the helper factor encoding sequence in 1 or 2 expression cassettes. In some embodiments, the host cell may comprise at least 3, 4 or 5 copies of the helper factor encoding sequence in 1, 2, 3, 4, or 5 expression cassettes. In some embodiments, the host cell may comprise at least 3, 4, 5, 6, 7, 8, 9, 10, 11, 12, 13, 14, 15 or 16 copies of the heterologous encoding sequence in 1, 2, 3, 4, 5, 6, 7, 8, 9, 10, 11, 12, 13, 14, 15 or 16 expression cassettes.

In some embodiments, the heterologous protein may be a food-related protein. In some embodiments, the food-related protein may comprise an enzyme, a nutritive protein, a food ingredient or a food additive. In some embodiments, the food-related protein may be a pepsinogen protein. In some embodiments, the copy number ratio of the helper factor encoding sequence to pepsinogen encoding sequence may be from 1:2 to 1:5.

In some embodiments, the food-related protein may comprise an egg-white protein. In some embodiments, the egg-white protein may be ovomucoid. In some embodiments, a copy number ratio of the helper factor encoding sequence to ovomucoid encoding sequence may be from 1:3 to 1:6. In some embodiments, the egg-white protein may be ovalbumin. In some embodiments, a copy number ratio of the helper factor encoding sequence to ovalbumin encoding sequence may be from 1:3 to 1:8.

In some embodiments, the engineered host cell may be capable of producing at least about 5 g per liter of the heterologous protein under fermentation conditions. In some embodiments, the engineered host cell may be capable of producing at least about 10 g per liter of the heterologous protein under fermentation conditions. In some embodiments, the engineered host cell may be capable of producing at least about 20 g per liter of the heterologous protein under fermentation conditions.

In some embodiments, the at least one of the expression cassettes may comprise a secretion signal. In some embodiments, the at least one of the expression cassettes may comprise a terminator sequence. In some embodiments, each of the helper factor gene sequences encodes for a protein independently selected from the group consisting of HAC1, Serine/threonine protein kinase 2 (Kin2), squalene synthase (ERG9), protein disulfide isomerase 1 (PDI1), SSA1, SSA4, SSB1, SSE1, BiP, ER Membrane Protein Complex Subunit 1 (EMC1), YNL181W oxidoreductase, integral membrane protein zinc metalloprotease Ste24, 14-3-3 protein Bmh2 and ER oxidoreductin 1 (Ero1).

In some embodiments, the host cell may be engineered to favor non-homologous integration over homologous integration. In some cases, the host cell is selected based on a greater number of non-homologous integrations than homologous integrations. In some embodiments, the at least two of the expression cassettes may comprise the heterologous gene sequence integrate at different integration sites. In some embodiments, the host cell may be a yeast cell. In some embodiments, the yeast cell may be Pichia pastoris. In some embodiments, the copy number may be measured by sequencing the host cell genome.

In some aspects, provided herein are methods of producing a recombinant heterologous protein in a host cell wherein the method may comprise: transforming a first vehicle into the host cell; wherein said first vehicle may comprise one or more first expression cassettes; wherein each of the first expression cassettes may comprise at least a first promoter operably linked to a heterologous gene sequence encoding the heterologous protein; allowing random integration of the one or more first expression cassettes into the host cell. In some embodiments, the method comprises identifying the integration of the one or more first expression cassettes into the host cell; transforming a second vehicle into the host cell; wherein said second vehicle may comprise one or more second expression cassettes; wherein each of the second expression cassettes may comprise at least a second promoter operably linked to the heterologous gene sequence; wherein the second promoter may be different from the first promoter. In some embodiments, the method comprises allowing random integration of the one or more second expression cassettes into the host cell and wherein the host cell may be a yeast or filamentous fungi, and wherein the engineered cell may be capable of producing at least 5 g per liter of the heterologous protein under fermentation conditions.

In some embodiments, the method may comprise transformation of a plurality of vehicles in addition to the second vehicle, wherein each of the plurality of vehicles comprise one or more expression cassettes each may comprise one or more promoter driving expression of the heterologous gene sequence encoding the heterologous protein. In some embodiments, the one or more promoters comprise the first promoter, the second promoter or a combination thereof. In some embodiments, the one or more promoters comprise the first promoter, the second promoter, promoters other than the first or second promoter or a combination thereof.

In some embodiments, the identifying the integration of the one or more first expression cassettes comprise sequencing a nucleic acid obtained from the host cell. In some embodiments, the identifying the integration of the one or more first expression cassettes may comprise identifying the presence or absence of a resistance marker; wherein the first expression cassette or the first plasmid may comprise a sequence encoding the resistance marker.

In some embodiments, the heterologous protein may be secreted into the medium during fermentation and wherein the heterologous recombinant protein may be harvested from the fermentation media. In some embodiments, the first expression cassette and the second expression cassette are linear molecules, and where the first expression cassette and the second expression cassette comprise less than 700 bp at the 5′ end with homology to a native host cell genomic locus.

In some embodiments, the host cell may be engineered to favor non-homologous integration over homologous integration. In some cases, the host cell may be selected based on a greater number of non-homologous integrations than homologous integrations In some embodiments, the method further may comprise transformation of a helper vehicle into the host cell; wherein said helper vehicle may comprise one or more helper expression cassettes; wherein each of the helper expression cassettes may comprise at least one promoter operably linked to a gene sequence encoding a helper factor protein. In some embodiments, a promoter in the one or more helper expression cassettes may be the same as the first or second promoter. In some embodiments, a promoter in the one or more helper expression cassettes may be the different from the first or second promoter. In some embodiments, the vehicle may be a plasmid. In some embodiments, the vehicle may be a linearized plasmid.

In some aspects, described herein is an engineered cell for producing a recombinant food-related protein by fermentation, comprising: at least one first cassette comprising a first promoter operably linked to a first gene encoding a first heterologous protein; and at least one second cassette comprising a second promoter operably linked to a second gene encoding the first heterologous protein; where the first and second cassettes are integrated at or near the same genomic locus of a host cell locus to produce the engineered cell; wherein the genomic locus does not share significant sequence homology with any of the first promoter, second promoter, first gene or second gene, and wherein the host cell is a yeast or filamentous fungi, and wherein the engineered cell is capable of producing at least 5 g per liter of the heterologous protein under fermentation conditions. In some embodiments, the first and second promoters are different from one another.

In embodiments, the host cell is a methylotrophic organism. In embodiments, the host cell is Komagataella phaffii or Komagataella pastoris. In embodiments, the engineered cells can comprise 2-5 copies of the first expression cassette. In other embodiments, the engineered cell can comprise 2-5 copies of the second expression cassette. In embodiments, the first heterologous protein comprises an animal-derived protein sequence. In some embodiments, the animal-derived protein sequence encodes an egg white protein. In embodiments, the egg white protein is selected from ovomucoid (OVD), ovalbumin (OVA), ovotransferrin and lysozyme. In one aspect, the first heterologous protein comprises pepsinogen. In embodiments, the first cassette and the second cassette are integrated into the genome of the engineered cell in the same 5′ to 3′ orientation. In embodiments, the first cassette and the second cassette are integrated into the genome of the engineered cell in an opposite 5′ to 3′ orientation.

In embodiments, the engineered cell can further comprise at least one third cassette integrated into the genome. In embodiments, the third cassette comprises a third promoter operably linked to a third gene. In embodiments, the third gene encodes a helper factor. In embodiments, the third gene encodes the first heterologous protein. In embodiments, the third gene encodes the second heterologous protein. In embodiments, the third cassette is integrated in the genome of the engineered cell at an integration site different from that of the first cassette and second cassette. In another aspect, a third cassette is integrated at the same genomic locus as the first cassette and the second cassette.

In embodiments, the first promoter is an inducible promoter. In embodiments, the second promoter is an inducible promoter. In embodiments, the first promoter is a constitutive promoter. In embodiments, the second promoter is a constitutive promoter. In embodiments, the inducible promoter is methanol inducible. In embodiments, the methanol inducible promoter is selected from the group consisting of AOX1, AOX2, FDH, FLD1, PEX11, DAS and a methanol inducible fragment thereof. In embodiments, the constitutive promoter is selected from the group consisting of GAP, GCW14. In embodiments, a first secretion signal is operably linked to the protein encoded by the first gene. In embodiments, a second secretion signal is operably linked to the protein encoded by the second gene.

In embodiments, provided here is a method of fermenting high titers of a heterologous recombinant protein, comprising providing the engineered cell in a fermentation culture; growing the fermentation culture to a minimum density of 50 grams of dry cell weight for a protein concentration of at least 2 to 10 grams of protein per liter within 2 to 12 days of fermentation; and harvesting the heterologous recombinant protein, wherein the heterologous recombinant protein is encoded by the first gene and the second gene of the engineered cell. In embodiments, the titer of the recombinant protein reaches at least 4 g protein per 50 g of dry cell weight per liter within 2-12 days. In embodiments, the heterologous recombinant protein is secreted into the medium during fermentation, and the heterologous recombinant protein is harvested from the fermentation media.

In embodiments, the heterologous recombinant protein comprises an animal derived protein. In embodiments, the animal derived protein is a food-related protein, such as a food ingredient, a food component or an enzyme useful for food processing and production. In embodiments, the animal-derived protein comprises an egg white protein, and the produced egg white protein displays one or more functional characteristics of native whole egg or egg white. In embodiments, at least one of the functional characteristics of the produced egg white protein is equivalent to or improved over the functional characteristics of native whole egg or egg white. In embodiments, the one or more functional characteristics are selected from the group consisting of solubility, clarity, texture, foaming, whipping, seeping, gelling, clarification, coagulation, coating, crystallization control, drying, edible packaging film, finishing, flavor, fortification, freezability, gloss, humectancy, insulation, moisturizing, mouthfeel, pH stability, protein enrichment, richness, shelf life extension, structure, tenderization, texture, thickening, water-binding, oil-binding, browning, emulsification, nitrogen:carbon ratio and/or anti-microbial activity.

In some embodiments, the animal derived protein expressed using the cassettes and methods herein comprises an enzyme. In embodiments, the enzyme is used in food-related processes, for example the enzyme is trypsin, chymotrypsin, lysozyme, pepsin or a pre- or pre-pro-form thereof.

In embodiments, provided here is a method of producing an engineered cell, comprising: transforming the host cell with a nucleic acid composition comprising the first cassette and the second cassette, where the first cassette and the second cassette are not covalently linked in the nucleic acid composition. In embodiments, the first cassette and the second cassette are linear molecules, where the first cassette and the second cassette comprise less than 700 bp at the 5′ end with homology to a native host cell genomic locus. In embodiments, the host cell is engineered to favor non-homologous integration over homologous integration.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the generation of engineered OVD transformants by homologous vs ectopic integration. Two strains already expressing OVD were transformed with more copies of OVD using two plasmids, one containing 3 copies of OVD, one containing 6 copies of OVD and which were designed to target these copies to a specific locus. FIG. 1 compares the relative protein expression in engineered OVD strains by homologous vs ectopic expression. 5 to 6 single colonies from PCR-checked transformants of the strains CF14 and CF15 were rescreened. FIG. 1A and FIG. 1B show a summary of CF14 and CF15 rescreens from this experiment.

### DETAILED DESCRIPTION

Provided herein are biological systems and methods for high production of animal-derived proteins, such as animal-derived food-related proteins using regulatory control in engineered methylotrophic yeast cells such as Pichia sp. (also known as and referred to herein in the alternate as Komagataella sp.). The biological systems and methods described herein employ non-homologous integration of heterologous gene sequences and in some cases, use these integration sites to stack expression cassettes for heterologous gene expression. In some embodiments, the integrated heterologous sequence encodes for a food-based or food-related protein, such as one that is used as a food ingredient or during manufacturing to make a food product. The systems and methods herein provide high levels of gene expression, leading to high titers of protein expression (greater than 5 g/L) in fermentation conditions, particularly larger-scale fermentation conditions.

The following disclosure describes systems and methods of driving high expression of a heterologous protein in a host cell by combining (i) stable integration of a plurality of expression cassettes driven by a diverse set of promoters, each integration site carrying a plurality of copies of one or more expression cassettes; (ii) co-transformation of the multiple expression cassettes into a single site or in the vicinity of a single site in the genome of a host cell, preferably a Pichia pastoris host cell, using non-homologous recombination methods; and, optionally, (iii) removal of antibiotic or other selection markers post-integration of the expression cassettes in the host cell genome. The integration of expression cassettes with diverse promoters can overcome potential issues with multiple copy integration such as possible depletion of cognate transcription factors that are required for the expression of the cassettes and the potential for deletion of copies through recombination events or other host mechanisms.

The systems and methods provided herein are designed to promote integration at non-homologous sites in the host genome. Unlike some yeasts that favor homologous recombination, Pichia sp. favors non-homologous integration of heterologous sequences. Despite this favored mechanism, most expression systems for Pichia sp. utilize homologous integration of heterologous genes. Surprisingly, a comparison of engineered cells with different integration events at a smaller scale (such as a test tube or shake flask setting) showed nearly equivalent protein production levels, but when compared at larger scale fermentation production formats, higher levels of the desired heterologous protein expression were noted in P. pastoris cells with non-homologous integration events of the corresponding transgenes.

In some embodiments, after integration, the engineered cells described herein do not contain sequences encoding selectable markers such as auxotrophic markers or antibiotic resistance genes, thereby reducing the amount of extraneous heterologous DNA that is integrated into the host genome. Additionally, because many auxotrophic markers are highly homologous to endogenous genes in the host cell, the use of such markers may favor homologous recombination of the transformed DNA.

The systems and methods herein are particularly useful for producing nutritive proteins, e.g., plant or animal proteins for food ingredients and products, with applications in food and health, as well as animal-derived proteins for food production, because of the improved capability for high-titer expression in large-scale settings as well as a “cleaner” production system without the utilization of antibiotic or other selection markers.

### I. HIGH-TITER PRODUCTION OF RECOMBINANT FOOD PROTEINS

The methods herein provide for improved high-titer production of recombinant protein from engineered host cells in a high-volume growth format, such as in a fermentation tank.

In some embodiments, the methods herein include heterologous protein production from engineered host cells in a large-scale growth settings at culture volumes of greater than about 1, 2, 3, 5, 10, 20, 50, 100, 500, 1000 liters and over time periods such as 1, 2, 3, 4, 5, 6, 7, 8, 9, 10 or more than 10 days. The systems and methods herein provide titers of the desired protein under fermentation conditions of at least 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48 or 50 g protein/liter of culture media in a large scale growth format e.g., fermentation tanks. The desired titers of the heterologous protein can be reached over time periods such as 6 hours, 12 hours, 18 hours, 24 hours, 48 hours or 72 hours. In some cases, the desired titers of the heterologous protein under fermentation conditions can be reached over time periods such as 1, 2, 3, 4, 5, 6, 7, 8, 9, 10 or more than 10 days. In some embodiments, such titers are the amounts of secreted desired protein from the fermentation culture. In some embodiments, such titers are the amounts of total desired protein (intracellular and extracellular) present in the fermentation culture. In some embodiments, such titers are the amounts of secreted protein from the fermentation culture.

In some embodiments, the methods herein include heterologous recombinant protein production from engineered host cells reaching culture densities of up to 10 grams of cells per liter of culture media, 30 g/L, 40 g/L, 50 g/L, 70 g/L, 100 g/L or 150 g/L. In some embodiments, the methods herein include heterologous recombinant protein production from engineered host cells reaching cell densities of up to 100 g dry cell weight/L, 150 g dry cell weight/L or 200 g dry cell weight/L.

### Fermentation Conditions

The methods herein provide for fermentation conditions that provide improved high-titer production of heterologous proteins from engineered host cells in a high-volume growth format, such as in a fermentation tank. Yeast strain glycerol stocks are thawed and inoculated at a 0.2% inoculum ratio in baffled shake flasks containing BMDY media (BMDY media is similar to BMGY media, with the glycerol, ‘G’, having been replaced with glucose/dextrose, ‘D’, Pichia Easy Select Manual, Thermo Fisher). Shake flasks are left to incubate at 30° C. and 250 rpm for 26 hrs. Shake flask cultures are then transferred at a 10% ratio to bioreactors containing BSM (basal salt medium), glucose, and trace metals (Pichia Fermentation Process Guidelines, Thermo Fisher).

The bioreactor fermentation is divided into three phases. During phase 1, the culture may be grown for 24 hrs until all glucose is consumed. During phase 2, the culture may be fed glucose at a glucose-limiting rate for 12 hours. In phase 3, the culture may be induced by continuously feeding a co-feed of glucose and an activator of a inducible promoter (e.g., methanol for the AOX1 promoter or PEX11 promoter) for 96 hours.

In one embodiment, the invention provides a method of improving the volumetric productivity of a recombinant protein of interest from host cells under fermentation culture conditions. In embodiments, the invention provides a cell culture medium optimized for use in a methanol inducible fermentation system (e.g., under the control of the AOX1 promoter) for the production of a recombinant protein of interest in yeast host cells using a fed-batch fermentation process. In embodiments, the invention provides a cell culture medium optimized for use in a methanol inducible fermentation system (e.g., under the control of the AOX1 promoter) for the production of a recombinant protein of interest in yeast host cells using a continuous fermentation process. In some cases, the host cell is a yeast Pichia cell.

In embodiments, the method comprises a) providing a glycerol fed yeast host cell culture comprising Pichia cells that are engineered as described elsewhere herein b) providing a methanol fed medium, and optionally an osmoprotectant, and c) inducing the yeast host cells under fermentation conditions to allow expression of the recombinant protein wherein the volumetric productivity of the protein of interest is higher than at least 5 g/L. As used herein the term “volumetric productivity” means the amount of target recombinant protein per unit volume of culture (g/L). In some embodiments, optimization of fermentation conditions can be used to improve the volumetric productivity of the Pichia strains engineered as described herein by 20%, 25%, 30%, 35%, 40%, 45%, 50%, 55%, 60%, 65%, 70%, 75%, 80%, 85%, 90%, 95% or more than 100%.

In some cases, a seed culture of the host cell engineered as described herein is inoculated into a starter culture composed of suitable culture medium. In some cases, the medium is BMGY medium. In some cases, the medium is BMDY media. In some cases, the volume of the starter culture medium is up to 200 ml, up to 300 ml, or up to 500 ml. In some cases, the starter culture is incubated at a temperature of 24° C., 25° C., 26° C., 27° C., 28° C., 29° C., 30° C., 31° C. or 32° C. In some cases, the starter culture is incubated for up to 6 hours, 12 hours, up to 24 hours, up to 36 hours or up to 48 hours. In some cases, the starter culture is shaken during incubation at 100 rpm, 200 rpm, 300 rpm, 500 rpm or 600 rpm. In some cases, a bioreactor system providing fermentation conditions for cultivation of host cells, is inoculated with a volumetric ratio of seed to initial fermentation medium of up to 3%, up to 5%, up to 10%, up to 15% or up to 20%. In some cases, the initial fermentation medium is BMGY medium. In some cases, the initial fermentation medium is BSM medium (basal salt medium). In some cases, the initial fermentation medium contains glucose and trace metals.

In embodiments, methanol inducible fermentation systems, based on the AOX1 promoter, can use glycerol as a substrate for biomass growth, followed by a methanol feed for induction of heterologous protein expression. In embodiments, cultivation of Pichia cells under fermentation conditions involves a multistage fermentation process. In embodiments, the multistage process is a batch fed process. In embodiments, the initial stage can include a glucose fed phase where the cells are cultured in a glucose-containing medium to accumulate biomass. In some cases, the initial stage can include a glycerol fed phase where the cells are cultured in a glycerol-containing medium to accumulate biomass.

In embodiments, in the next stage, the cells can be fed glucose at a rate limiting rate to prepare for induction phase. In embodiments, the rate limiting feeding rate of glucose can range from up to 0.005 g/l, up to 0.05 g/l, or up to 0.5 g/1 per hour of specific growth rate. In some cases, glucose can be fed for up to 8 hours, up to 10 hours, up to 14 hours, up to 16 hours, up to 20 hours, or up to 24 hours, up to 30 hours, up to 36 hours, up to 40 hours, or up to 48 hours. In some cases, host cells can be fed with glycerol instead of before methanol induction.

In some cases, the methanol induction phase can be preceded by a starvation phase. In some cases, the starvation phase before induction can last for 30 minutes, up to 60 minutes, up to 90 minutes, up to 120 minutes, up to 150 minutes, up to 180 minutes, up to 4 hours, up to 6 hours, up to 8 hours, up to 9 hours, up to 10 hours, up to 15 hours, up to 18 hours, up to 20 hours.

In some cases, methanol feed rate can be optimized to improve production of recombinant protein production in host cells. In some cases, methanol feeding regimes, for example, maintaining a fixed methanol concentration (Damasceno et al, 2004), controlling dissolved oxygen concentration with methanol feed rate (Charoenrat et al, 2005), carbon limited feed strategies (Zhang et al, 2000) as well as mixed carbon source feeds (Ramon et al, 2007) can be used for increasing the rate of production of heterologous protein from engineered host cells. In some cases, methanol can be continuously fed at a constant rate. In some cases, the methanol feed rate can be up to 0.5 g/L/h, up to 0.7 g/L/h, 0.8 g/L/h, 0.9 g/L/h, 1.1 g/L/h, 1.3 g/L/h, 1.5 g/L/h, 1.6 g/L/h, 1.8 g/L/h, 1.9 g/L/h, 2.1 g/L/h, 2.4 g/L/h, 2.6 g/L/h, 2.7 g/L/h, 2.9 g/L/h, 3.1 g/L/h, 3.3 g/L/h, 3.5 g/L/h, 3.7 g/L/h, 3.9 g/L/h, 4.5 g/L/h or 5.0 g/L/h. In some cases, methanol can be fed at an exponential rate. In some cases, methanol can be added as a periodic bolus. In some case, host cells are co-fed glucose along with methanol. In some cases, the glucose feeding rate can be up to 0.5 g/L/h, up to 0.7 g/L/h, 0.8 g/L/h, 0.9 g/L/h, 1.1 g/L/h, 1.3 g/L/h, 1.5 g/L/h, 1.6 g/L/h, 1.8 g/L/h, 1.9 g/L/h, 2.1 g/L/h, 2.4 g/L/h, 2.6 g/L/h, 2.7 g/L/h, 2.9 g/L/h, 3.1 g/L/h, 3.3 g/L/h, 3.5 g/L/h, 3.7 g/L/h, 3.9 g/L/h, 4.5 g/L/h or 5.0 g/L/h.

In some cases, the length of methanol induction phase can be up to 1 day, up to 2 day, up to 3 days, up to 4 days, up to 5 days, up to 6 days, up to 7 days, up to 8 days, up to 9 days, or up to 10 days. In some cases, the length of methanol induction phase can be at least 1 day, at least 2 day, at least 3 days, at least 4 days, at least 5 days, at least 6 days, at least 7 days, at least 8 days, at least 9 days, or at least 10 days.

Suitable culture media can be designed to provide pure carbon sources. In some cases, the media can optionally provide biotin, salts trace elements and water. In some cases, the carbon source for the host cells can be selected from glucose, fucose, mannose, sorbose, or glycerol, sorbitol. In some cases, the medium can be BSGY, BMGY, BMMY, MD, or YPD medium. In some cases, the medium composition can influence heterologous protein expression in host cells by affecting cell growth and viability or altering the secretion of extracellular proteases. In some cases, sorbitol or betaine can be added to culture media to increase production of the heterologous recombinant protein. In embodiments, the addition of an organic nitrogen source (e.g., a mixture of yeast extract and peptone) to a fed-batch culture system can be used to increase heterologous protein production in host yeast cells.

Cell wall integrity of the host cells can affect the production yield of the heterologous protein. In embodiments, improved culture conditions utilizing optimized media and fermentation conditions can be designed to improve the cell well integrity of the engineered Pichia strains. For example, in embodiments, the fermentation medium can comprise a basal medium supplemented with a non-fermentable sugar or a non-fermentable sugar alcohol as an osmoprotectant. In particular embodiments, the osmoprotectant can be selected from maltose, sorbose, ribose, maltitol, myo-inositol, mellibiose, and quinic acid. In some cases, glycerol, arabitol, glycine betaine, sorbitol or trehalose can be utilized for modulating cellular osmotic pressure under osmotic stress conditions. The osmoprotectants can be added to any suitable basal medium. In particular embodiments the osmoprotectant can be added in addition to other media supplements, including, but no limited to mixes comprising amino acids, vitamins, trace metals or basal salts. In embodiments, the inclusion of the osmoprotectant can be maintained through the glycerol feeding phase, the methanol induction phase or both.

In embodiments, the osmoprotectant is present at concentration of about 15 g/L, about 25 g/L, about 35 g/L, about 50 g/L, about 75 g/L or about 100 g/L. In embodiments, the presence of the osmoprotectant in the batch media increases and maintains the osmolality of the batch media at more than about 50 mOsm/kg, more than about 100 mOsm/kg, more than about 200 mOsm/kg, more than about 500 mOsm/kg, more than about 700 mOsm/kg, more than 1000 mOsm/kg, or more than about 1500 mOsm/kg. In embodiments, increased osmolality is maintained from about 24 hours to about 48 hours, to about 80 hours to about 110 hours or until completion of the methanol induction phase (e.g., ranging from about 24 to about 150 hours). In some cases, the increased osmolality is maintained through the methanol feeding phase.

In some cases, cultivation parameters e.g., pH, temperature or dissolved oxygen can be optimized to improve production of recombinant protein production in host cells. In some cases, the cultivation temperature conditions can be at least 24° C., 24.1° C., 24.2° C., 24.5° C., 24.8° C., 26.0° C., 26.3° C., 26.5° C., 26.8° C., 27.0° C., 27.2° C., 27.5° C., 27.8° C., 29.0° C., 29.3° C., 29.5° C., 29.8° C., 30.0° C., 30.3° C., 30.5° C., 30.7° C., 31° C., 31.3° C., 31.5° C., 31.7° C., 31.9° C., 32.3° C., 32.6° C., 32.8° C., 33.0° C., 33.1° C., 33.5° C., 33.6° C. or 34.0° C. In some cases, the pH of the fermentation cultivation conditions can be up to 5, 5.2, 5.4, 5.6, 5.8, 6.0, 6.2, up to 6.4, up to 6.6, up to 6.7, up to 6.8, up to 6.9, up to 7.0, up to 7.1, up to 7.3, up to 7.5, up to 7.8, up to 7.9, or up to 8.0. In some cases, the pH of the fermentation cultivation conditions can be at least 4, 4.4, 4.6, 4.8, 5, 5.2, 5.4, 5.6, 5.8, 6.0, 6.2, 6.4, 6.6, 6.7, 6.8, 6.9, 7.0, 7.1, 7.3, 7.5, 7.8 or 7.9. In some cases, dissolved oxygen levels can be maintained at up to 15%, up to 17%, up to 20%, up to 22%, up to 25%, up to 27%, up to 30%, up to 32% or up to 35% of saturation.

### Food Proteins

In some embodiments, the methods provided herein can be used for the production of animal-derived food-related proteins in a large-scale fermentation setting. In some cases, the animal-derived protein is an enzyme, such as used in manufacturing, processing and/or production of food and/or beverage ingredients and products. Some examples of animal-derived enzymes including trypsin, chymotrypsin, pepsin and pre- and pre-pro-forms of such enzymes; as an example, pepsinogen is the pre-/pre-pro-form of pepsin. In some cases, the animal protein is a nutritive protein such as a protein that holds or binds to a vitamin or mineral (e.g., an iron-binding protein or heme binding protein), or a protein that provides a source of protein and/or particular amino acids.

In some embodiments, the methods provided herein can be used for the production of food proteins in a large-scale fermentation setting. In some cases, the food protein can be an animal protein. In some embodiments, the animal protein can be an egg-related protein. Exemplary examples of such egg white proteins can be ovalbumin (OVA), ovomucoid (OVD), ovotransferrin, and lysozyme proteins. Other examples of egg-related proteins include ovomucin, ovoglobulin G2, ovoglobulin G3 and any combination thereof. Additional examples of egg-related proteins include ovoinhibitor, ovoglycoprotein, flavoprotein, ovomacroglobulin, ovostatin, cystatin, avidin, ovalbumin related protein X, ovalbumin related protein Y and any combination thereof.

In some cases, the protein produced using the systems and methods provided herein is post-translationally modified. Such modifications include glycosylation and phosphorylation. In some cases, the post-translational modification of the produced protein is the same or substantially similar to the natively produced protein. In some cases, the post-translational modification of the produced protein is altered as compared to the native source of the protein.

In some embodiments, the recombinant protein harvested using the systems and methods provided herein may provide at least one or more functional characteristics of the native protein. For example, recombinant egg-white ovalbumin protein can exhibit at least one or more functional characteristics of native egg-white proteins, selected from the group consisting of gelling, foaming, whipping, fluffing, binding, springiness, aeration, creaminess, and cohesiveness to the composition. In other cases, the one or more functional characteristics can be selected from the group consisting of solubility, clarity, texture, foaming, whipping, seeping, gelling, nitrogen:carbon ratio, water-binding, oil-binding, browning, emulsification, clarification, coagulation, coating, crystalization control, drying, edible packaging film, finishing, flavor, fortification, freezability, gloss, humectancy, insulation, moisturizing, mouthfeel, pH stability, protein enrichment, richness, shelf life extension, structure, tenderization, Texture, thickening, or anti-microbial activity. In some cases, the recombinant animal protein harvested using the systems and methods provided herein may provide at least one or more functional characteristics that is substantially the same or better than the same characteristic of the native protein. In one example, characteristics of recombinant ovalbumin produced with the systems and methods herein may be substantially the same or better than the same characteristic provided by native egg white. In some embodiments, provided herein is a recombinant protein composition for use as an egg-white replacer.

The proteins produced using the systems and methods herein can be used in food ingredients and food products. For example, recombinant ovalbumin produced using the methods described herein can provide one or more functional features to food ingredients and food products. In some cases, the recombinant animal protein produced using the methods herein, can provide a nutritional feature such as protein content, protein fortification and amino acid content to a food ingredient or food product. For example, the nutritional feature provided by recombinant ovalbumin produced using the methods herein, may be comparable or substantially similar to an egg, egg white or native ovalbumin. In other cases, the nutritional feature provided by recombinant ovalbumin produced using the methods herein, may be better than that provided by a native egg or native egg white.

Food compositions can include the recombinant food proteins, e.g., recombinant ovomucoid, in an amount between 0.1% and 50% on a weight/weight (w/w) or weight/volume (w/v) basis. Recombinant proteins produced using the systems and methods herein, may be present in food compositions at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45% or 50% on a weight/weight (w/w) or weight/volume (w/v) basis. Additionally, or alternatively, the concentration of recombinant proteins produced using the systems and methods herein, may be present in such food compositions is at most 70%, 60%, 50%, 40%, 30%, 20%, 15%, 10%, 5%, 4%, 3%, 2% or 1% on a w/w or w/v basis. In some embodiments, the recombinant protein in the food ingredient or food product can be at a concentration range of 0.1%-50%, 1%-30%, 0.1%-20%, 1%-10%, 0.1%-5%, 1%-5%, 0.1%-2%, 1%-2% or 0.1-1% w/w.

### II. GENERATION OF ENGINEERED CELL FOR HIGH-YIELD PRODUCTION OF FOOD PROTEINS

The systems and methods provided herein are designed for engineering of a host cell by introducing into the host cell, heterologous sequences for recombinant protein expression comprised within one or more expression cassettes.

One or more expression cassettes may be integrated into a host cell. A host cell can comprise a first expression cassette. The first expression cassette can have a first promoter operably linked to a first gene encoding a first heterologous protein. The host cell can comprise a second expression cassette. In some cases, the first expression cassette and the second expression cassette encode the same protein. For example, the first expression cassette and the second expression cassette can drive the expression of a recombinant ovomucoid protein. In some cases, the recombinant heterologous protein expressed in the first and the second expression cassette are encoded by the same gene sequence. In some cases, the recombinant protein expressed in the first and the second expression cassette can be encoded by different gene sequences. For instance, the expression cassettes may comprise one or more gene sequences encoding for the same protein such as, one of the gene sequences may be codon optimized. In some cases, the gene sequence encoding the recombinant protein expressed in the first and the second expression cassette can have sequence similarity of at least 80%, at least 85%, at least 90%, at least 95% or at least 99%.

In some cases, the recombinant protein expressed in the first expression cassette can be a homologous protein to the recombinant protein in the second expression cassette. For instance, the recombinant protein in the first expression cassette can be from an egg-related protein from a first species and the recombinant protein in the second expression cassette can be a homologous egg-related protein from a related species. For example, the recombinant protein in the first expression cassette can be an ovomucoid protein encoded by the Gallus gallus domesticus and the recombinant protein in the second expression cassette can be an ovomucoid protein encoded by the Anas platyrhynchos species. In some cases, the homologous gene sequences encoding the recombinant protein expressed in the first and the second expression cassette can have sequence similarity of at least 80%, at least 85%, at least 90%, at least 95% or at least 99%.

In some cases, the first expression cassette and the second expression cassette can encode different proteins. For example, the first expression cassette and the second expression cassette can drive the expression of an ovomucoid and an ovalbumin protein respectively. In some cases, optionally a third expression cassette can be operably linked to a third gene. In some cases, the third gene can encode the first recombinant protein. In some cases, the third gene can encode the second recombinant protein. In some cases, optionally the third gene encodes a third recombinant protein. In some cases, a third recombinant heterologous protein can encode a helper protein, i.e., a protein that aids in the expression of the first or the second heterologous protein.

In some cases, optionally a fourth expression cassette can be operably linked to a fourth gene. In some cases, the fourth gene can encode the first recombinant protein. In some cases, the fourth gene can encode the second recombinant protein. In some cases, optionally the fourth gene encodes a third recombinant protein. In some cases, optionally a fifth expression cassette can be operably linked to a fifth gene. In some cases, the fifth gene can encode the first recombinant protein. In some cases, the fifth gene can encode the second recombinant protein. In some cases, optionally the fifth gene encodes the third recombinant protein. In some cases, optionally the fifth gene encodes a fourth recombinant protein. In some cases, optionally the fifth gene encodes a fifth recombinant protein.

In some cases, the recombinant heterologous proteins encoded by the first or the second expression cassettes can be animal-derived proteins. In some cases, the animal-derived proteins are food-related proteins. In some cases, the animal-derived proteins can be egg-related proteins. Examples of egg-related proteins or egg-white proteins include for example, ovomucoid, ovalbumin, lysozyme, ovotransferrin, ovomucin, ovoglobulin G2, ovoglobulin G3 and any combination thereof. In some cases, the sequence identity of the signal peptides may be a sequence having at least 80%, 90%, 95%, 96%, 97%, 98%, 99% or 99.5% sequence identity to SEQ ID NOs: 13-16 set forth in Table No. 4. Additional egg-related proteins for production include ovoinhibitor, ovoglycoprotein, flavoprotein, ovomacroglobulin, ovostatin, cystatin, avidin, ovalbumin related protein X, ovalbumin related protein Y and any combination thereof.

In some cases, the recombinant heterologous proteins encoded by the first or the second expression cassettes can be plant-based food proteins. In some cases, the one or more plant-based proteins may include, but are not limited to: pea protein isolates, and/or concentrates; garbanzo (chickpea) protein isolates, and/or concentrates; fava bean protein isolates, and/or concentrates; soy protein isolates, and/or concentrates; rice protein isolates, and/or concentrate; mung bean protein isolates, and/or concentrates; potato protein isolates, and/or concentrates; hemp protein isolates, and/or concentrates; or any combinations thereof. Plant-based proteins may include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. In particular embodiments, the pea proteins can be derived from yellow peas, such as Canadian yellow peas.

### Expression Cassette Integration Copy Number

In some embodiments, a vehicle or plasmid for integration into the host cell can comprise one or multiple copies of a first expression cassette. In some embodiments, a plasmid for integration into the host cell can comprise one or multiple copies of a first expression cassette and one or multiple copies of a second expression cassette. In some cases, an engineered host cell can integrate one or more plasmids, each plasmid comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a first expression cassette. In some cases, an engineered host cell can integrate one or more plasmids, each plasmid comprising at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a first expression cassette. In some cases, an engineered host cell can integrate one or more plasmids, each plasmid comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a second expression cassette. In some cases, an engineered host cell can integrate one or more plasmids, each plasmid comprising at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a second expression cassette.

In some cases, an engineered host cell can integrate one or more copies of a first expression cassette, one or more copies of a second expression cassette, and optionally one or more copies of a third expression cassette. In some cases, the host cell integration can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a first expression cassette and at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a second expression cassette. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of the third expression cassette. In some cases, the integration can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a first expression cassette and at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a second expression cassette. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a third expression cassette.

In some cases, an engineered host cell can integrate one or more copies of a gene sequence encoding a first recombinant protein, one or more copies of a gene sequence encoding a second recombinant protein, and optionally one or more copies of a transgene encoding a third recombinant protein. In some cases, the host cell integration can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a transgene encoding a first recombinant protein and at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a transgene encoding a second recombinant protein. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a transgene encoding a third recombinant protein. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a transgene encoding a fourth recombinant protein. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a transgene encoding a fifth recombinant protein.

An engineered host cell may comprise more than one copy of a heterologous gene encoding a heterologous protein such as an animal protein for recombinant production. A copy number of the heterologous gene integrated into a host cell genome may be determined using standard techniques such as quantitative PCR or sequencing. Techniques such as sequencing of the host cell genome may provide the least amount of variation in the copy number calculation and may therefore provide a more reliable copy number count. In some cases, the engineered host cell comprises 2 to 20 copies of the heterologous gene per cell. In some cases, the engineered host cell comprises at least 2 copies of the heterologous gene per cell. In some cases, the engineered host cell comprises at most 20 copies of the heterologous gene per cell. In some cases, the engineered host cell comprises 2 to 4, 2 to 5, 2 to 6, 2 to 8, 2 to 10, 2 to 12, 2 to 14, 2 to 16, 2 to 18, 2 to 20, 4 to 5, 4 to 6, 4 to 8, 4 to 10, 4 to 12, 4 to 14, 4 to 16, 4 to 18, 4 to 20, 5 to 6, 5 to 8, 5 to 10, 5 to 12, 5 to 14, 5 to 16, 5 to 18, 5 to 20, 6 to 8, 6 to 10, 6 to 12, 6 to 14, 6 to 16, 6 to 18, 6 to 20, 8 to 10, 8 to 12, 8 to 14, 8 to 16, 8 to 18, 8 to 20, 10 to 12, 10 to 14, 10 to 16, 10 to 18, 10 to 20, 12 to 14, 12 to 16, 12 to 18, 12 to 20, 14 to 16, 14 to 18, 14 to 20, 16 to 18, 16 to 20, or 18 to 20 copies of the heterologous gene per cell. In some cases, the engineered host cell comprises about 2, 4, 5, 6, 8, 10, 12, 14, 16, 18, or 20 copies of the heterologous gene per cell. In some cases, the engineered host cell comprises at least 2, 4, 5, 6, 8, 10, 12, 14, 16 or 18 copies of the heterologous gene per cell. In some cases, the engineered host cell comprises at most 4, 5, 6, 8, 10, 12, 14, 16, 18, or 20 copies of the heterologous gene per cell.

An engineered host cell may comprise one or more copies of a helper factor gene encoding a helper factor protein. A copy number of the helper factor gene integrated into a host cell genome may be determined using standard techniques such as quantitative PCR or sequencing. Techniques such as sequencing of the host cell genome may provide the least amount of variation in the copy number calculation and may therefore provide a more reliable copy number count. In some cases, the engineered host cell comprises 1 to 8 copies of a helper factor gene per cell. In some cases, the engineered host cell comprises at least 1 copy of a helper factor gene per cell. In some cases, the engineered host cell comprises at most 8 copies of a helper factor gene per cell. In some cases, the engineered host cell comprises 1 to 2, 1 to 3, 1 to 4, 1 to 5, 1 to 6, 1 to 7, 1 to 8, 2 to 3, 2 to 4, 2 to 5, 2 to 6, 2 to 7, 2 to 8, 3 to 4, 3 to 5, 3 to 6, 3 to 7, 3 to 8, 4 to 5, 4 to 6, 4 to 7, 4 to 8, 5 to 6, 5 to 7, 5 to 8, 6 to 7, 6 to 8, or 7 to 8 copies of a helper factor gene per cell. In some cases, the engineered host cell comprises about 1, 2, 3, 4, 5, 6, 7, or 8 copies of a helper factor gene per cell. In some cases, the engineered host cell comprises at least 1, 2, 3, 4, 5, 6 or 7 copies of a helper factor gene per cell. In some cases, the engineered host cell comprises at most 2, 3, 4, 5, 6, 7, or 8 copies of a helper factor gene per cell.

In some cases, a balanced ratio of copy numbers of a helper factor gene to a heterologous gene leads to an increase in the heterologous protein production. An overexpression of the heterologous gene in the absence of a helper factor protein may saturate the amount of protein that can be produced by the host cell but in some cases, the presence of one or more helper factor proteins, an engineered host cell may be able to overcome the saturation and further provide a higher titer. In some cases, an over expressed helper factor protein may lead to a lower protein production as well. In some cases, the host cell may comprise from 1.1, 1.2, 1.3, 1.5, 1.7, 1.9, 2, 2.2, 2.4, 2.5, 2.6, 2.8, 3, 3.2, 3.4, 3.6, 3.8, 4, 4.2, 4.4, 4.6, 4.8, 5, 5.4, 5.8, 6, 6.4, 6.8, 7, 7.4, 7.8, 8, 8.4, 8.8, 9, 9.4, 9.8, 10 or 12 copies of the heterologous gene per copy of the helper factor gene. In various embodiments, the copy number ratio of the helper factor encoding sequence to the heterologous protein encoding sequence is at least about 1:10, 1:9, 1:8, 1:7, 1:6, 1:5, 1:4, or 1:3. In some embodiments, the copy number ratio of the helper factor encoding sequence to the heterologous protein encoding sequence is at most about 1:9, 1:8, 1:7, 1:6, 1:5, 1:4, 1:3 or 1:2.

A balanced copy number ratio of helper factor gene to a heterologous gene may vary based on the heterologous protein and, without wishing to be bound by theory, a specific ratio may provide unexpectedly superior protein expression whereas another ratio (either above or below the specific ratio) may provide undesirable protein expression.

In one example, a copy number ratio of helper factor gene to an ovomucoid (OVD) gene may be from 1:2 to 1:8. In some examples, for each copy of the helper factor gene the host cell may comprise from 2 to 8 copies of the OVD gene. In some examples, for each copy of the helper factor gene the host cell may comprise at least 2 copies of the OVD gene. In some examples, for each copy of the helper factor gene the host cell may comprise at most 8 copies of the OVD gene. In some examples, for each copy of the helper factor gene the host cell may comprise from 2 to 2.25, 2 to 2.5, 2 to 2.75, 2 to 3, 2 to 3.5, 2 to 4, 2 to 4.5, 2 to 5, 2 to 5.5, 2 to 6, 2 to 8, 2.25 to 2.5, 2.25 to 2.75, 2.25 to 3, 2.25 to 3.5, 2.25 to 4, 2.25 to 4.5, 2.25 to 5, 2.25 to 5.5, 2.25 to 6, 2.25 to 8, 2.5 to 2.75, 2.5 to 3, 2.5 to 3.5, 2.5 to 4, 2.5 to 4.5, 2.5 to 5, 2.5 to 5.5, 2.5 to 6, 2.5 to 8, 2.75 to 3, 2.75 to 3.5, 2.75 to 4, 2.75 to 4.5, 2.75 to 5, 2.75 to 5.5, 2.75 to 6, 2.75 to 8, 3 to 3.5, 3 to 4, 3 to 4.5, 3 to 5, 3 to 5.5, 3 to 6, 3 to 8, 3.5 to 4, 3.5 to 4.5, 3.5 to 5, 3.5 to 5.5, 3.5 to 6, 3.5 to 8, 4 to 4.5, 4 to 5, 4 to 5.5, 4 to 6, 4 to 8, 4.5 to 5, 4.5 to 5.5, 4.5 to 6, 4.5 to 8, 5 to 5.5, 5 to 6, 5 to 8, 5.5 to 6, 5.5 to 8, or 6 to 8 copies of the OVD gene. In some examples, for each copy of the helper factor gene the host cell may comprise about 2, 2.25, 2.5, 2.75, 3, 3.5, 4, 4.5, 5, 5.5, 6, or 8 copies of the OVD gene. In some examples, for each copy of the helper factor gene the host cell may comprise at least 2, 2.25, 2.5, 2.75, 3, 3.5, 4, 4.5, 5, 5.5, 6 or 7 copies of the OVD gene. In some examples, for each copy of the helper factor gene the host cell may comprise at most 2.25, 2.5, 2.75, 3, 3.5, 4, 4.5, 5, 5.5, 6, or 8 copies of the OVD gene.

In some examples, for each copy of the helper factor gene the host cell may comprise 2 to 5 copies of the ovalbumin (OVA) gene. In some examples, for each copy of the helper factor gene the host cell may comprise at least 2 copies of the OVA gene. In some examples, for each copy of the helper factor gene the host cell may comprise at most 5 copies of the OVA gene. In some examples, for each copy of the helper factor gene the host cell may comprise from 2 to 2.5, 2 to 3, 2 to 3.2, 2 to 3.4, 2 to 3.6, 2 to 3.8, 2 to 4, 2 to 4.5, 2 to 5, 2.5 to 3, 2.5 to 3.2, 2.5 to 3.4, 2.5 to 3.6, 2.5 to 3.8, 2.5 to 4, 2.5 to 4.5, 2.5 to 5, 3 to 3.2, 3 to 3.4, 3 to 3.6, 3 to 3.8, 3 to 4, 3 to 4.5, 3 to 5, 3.2 to 3.4, 3.2 to 3.6, 3.2 to 3.8, 3.2 to 4, 3.2 to 4.5, 3.2 to 5, 3.4 to 3.6, 3.4 to 3.8, 3.4 to 4, 3.4 to 4.5, 3.4 to 5, 3.6 to 3.8, 3.6 to 4, 3.6 to 4.5, 3.6 to 5, 3.8 to 4, 3.8 to 4.5, 3.8 to 5, 4 to 4.5, 4 to 5, or 4.5 to 5 copies of the OVA gene. In some examples, for each copy of the helper factor gene the host cell may comprise about 2, 2.5, 3, 3.2, 3.4, 3.6, 3.8, 4, 4.5, or 5 copies of the OVA gene. In some examples, for each copy of the helper factor gene the host cell may comprise at least 2, 2.5, 3, 3.2, 3.4, 3.6, 3.8, 4 or 4.5 copies of the OVA gene. In some examples, for each copy of the helper factor gene the host cell may comprise at most 2.5, 3, 3.2, 3.4, 3.6, 3.8, 4, 4.5, or 5 copies of the OVA gene.

In some examples, for each copy of the helper factor gene the host cell may comprise 1.5 to 5 copies of the pepsinogen (PGA) gene. In some examples, for each copy of the helper factor gene the host cell may comprise at least 1.5 copies of the PGA gene. In some examples, for each copy of the helper factor gene the host cell may comprise at most 5 copies of the PGA gene. In some examples, for each copy of the helper factor gene the host cell may comprise from 1.5 to 1.75, 1.5 to 2, 1.5 to 2.25, 1.5 to 2.5, 1.5 to 2.75, 1.5 to 3, 1.5 to 3.5, 1.5 to 4, 1.5 to 5, 1.75 to 2, 1.75 to 2.25, 1.75 to 2.5, 1.75 to 2.75, 1.75 to 3, 1.75 to 3.5, 1.75 to 4, 1.75 to 5, 2 to 2.25, 2 to 2.5, 2 to 2.75, 2 to 3, 2 to 3.5, 2 to 4, 2 to 5, 2.25 to 2.5, 2.25 to 2.75, 2.25 to 3, 2.25 to 3.5, 2.25 to 4, 2.25 to 5, 2.5 to 2.75, 2.5 to 3, 2.5 to 3.5, 2.5 to 4, 2.5 to 5, 2.75 to 3, 2.75 to 3.5, 2.75 to 4, 2.75 to 5, 3 to 3.5, 3 to 4, 3 to 5, 3.5 to 4, 3.5 to 5, or 4 to 5 copies of the PGA gene. In some examples, for each copy of the helper factor gene the host cell may comprise about 1.5, 1.75, 2, 2.25, 2.5, 2.75, 3, 3.5, 4, or 5 copies of the PGA gene. In some examples, for each copy of the helper factor gene the host cell may comprise at least 1.5, 1.75, 2, 2.25, 2.5, 2.75, 3, 3.5 or 4 copies of the PGA gene. In some examples, for each copy of the helper factor gene the host cell may comprise at most 1.75, 2, 2.25, 2.5, 2.75, 3, 3.5, 4, or 5 copies of the PGA gene.

### Increased Protein Production

In some cases, the engineered cells and methods of use thereof of the present disclosure provide increased protein production relative to a control cell or control method.

In embodiments, the engineered cells and methods of use thereof provide about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, about 4-fold, or any fold therebetween increased protein production relative to a control cell or control method. In some embodiments, the engineered cells and methods of use thereof provide about 4.2-fold, 4.4-fold, 4.6-fold, 4.8-fold, 5-fold, 5.2-fold, 5.4-fold, 5.6-fold, 5.8-fold, 6-fold, 6.2-fold, 6.4-fold, 6.6-fold, 6.8-fold, 7-fold, 7.2-fold, 7.4-fold, 7.6-fold, 7.8-fold, 8-fold, 8.2-fold, 8.4-fold, 8.6-fold, 8.8-fold, 9-fold, 9.2-fold, 9.4-fold, 9.6-fold, 9.8-fold, about 10-fold, or any fold therebetween increased protein production relative to a control cell or control method. In various embodiments, the engineered cells and methods of use thereof provide about 10-fold, 15-fold, 20-fold, 25-fold, about 30-fold, or any fold therebetween increased protein production relative to a control cell or control method.

In some cases, the engineered cells and methods of use thereof provide from about 1-fold to about 2-fold, 2-fold to 3-fold, 3-fold to 4-fold, 4-fold to 5-fold, 5-fold to 6-fold, 6-fold to 7-fold, 7-fold to 8-fold, 8-fold to 9-fold, 9-fold to 10-fold, 10-fold to 15-fold, 15-fold to 20-fold, 20-fold to 25-fold, or from about 25-fold to about 30-fold increased protein production relative to a control cell or control method.

A control cell may be a cell that lacks a first expression cassette, a second expression cassette, and/or a third expression cassette as disclosed herein. A control cell may comprise a copy number of a heterologous protein encoding sequence that is less than or greater than the copy number disclosed herein. A control cell may comprise a copy number of a helper factor encoding sequence that is less than or greater than the copy number disclosed herein. A control cell may comprise a copy number ratio of a helper factor encoding sequence to a heterologous protein encoding sequence that is outside (i.e., greater than or lower than) the ratios disclosed herein. In some cases, a control may comprise any combination of the above-mentioned differences from the engineered cells disclosed herein. As examples, the copy number of the helper factor encoding sequence may be less than the copy number disclosed herein and the copy number ratio may be lower than the ratios disclosed herein or the control cell may lack a second expression cassette as disclosed herein and may comprise a copy number of a helper factor encoding sequence that is greater than the copy number disclosed herein.

### Promoter Diversity

It is possible that bottle necks of transcription arise from depletion of the pool of those cognate transcription factors that are available to mediate the activity of the integrated promoter in the one or more expression cassettes. In some embodiments, a variety of expression cassettes are introduced, with expression cassettes carrying different promoters to drive the transgene of interest, thus diversifying the demand on available transcription factors to drive expression. In some cases, each expression cassette carries a unique promoter that is different from the promoter carried by another expression cassette. Additionally, the use of multiple promoters reduces the homology between cassettes, which may increase stability of integration and copy number, particularly when multiple copies are integrated together at a site within the genome. In some cases, when an expression cassette that comprises a specific promoter is integrated into the cell's genome, should the cell be transformed with another expression cassette yet comprising the specific promoter, the homology of the integrated specific promoter and the transformed specific promoter may lead to homologous recombination and excision of the integrated expression cassette; in this case, rather than increasing copy number of integrated expression cassettes following a subsequent transformation, the copy number remains unchanged due to integration of a new cassette and excision of the old cassette.

In some embodiments herein, the first expression cassette and the second expression cassette contain different promoter sequences. The promoters can be derived from different sources (e.g. different regulatory regions). The promoters can be derived from the same or substantially similar sources but different in overall length of sequence and/or arrangement of regulatory elements. In some cases, the promoters can be synthetic promoters.

An engineered host cell may comprise more than one promoter operably linked to the sequence of the heterologous gene integrated into the genome. In some cases, an engineered host cell may comprise at least 2, 3, 4, 5, 6, 7 or 8 different promoters operably linked to sequences of one or more heterologous genes. In some cases, an engineered host cell may comprise at least 2, 3, 4, 5, 6, 7 or 8 different promoters operably linked to individual sequences of a heterologous gene. Each promoter linked to a gene may be transformed into the host cell using one plasmid or vehicle. Alternatively, promoters linked to genes may be transformed into the host cell using more than one plasmid or vehicle.

The promoter for a first expression cassette can be an inducible promoter. Inducible promoters include promoters that transcribe a gene's coding sequence when the inducer, such as a small molecule, protein, peptide, temperature, light or other environmental condition, is present; on the other hand, when the inducer is absent, there is little or no transcription and, therefore, protein expression. In some embodiments, an expression cassette includes an alcohol inducible promoter, such as a methanol inducible promoter. In some embodiments, such as where two or more different expression cassettes are employed in the systems and methods herein, each expression cassette can employ a different inducible promoter. In some embodiments, such as where two or more different expression cassettes are employed in the systems and methods herein, each expression cassette can employ the same inducible promoter. In some embodiments, the promoters of the first and the second expression cassettes are different promoter sequences, but are all inducible by the same inducer, such as for example, all methanol inducible promoters. Exemplary methanol inducible promoters for use in Pichia include AOX1, AOX2, FDH, PEX11, and sugar inducible promoters such as glucose-induced and rhamnose regulated promoters. Other examples of inducible promoters that can be included in the expression cassettes are described elsewhere in this disclosure.

A first expression cassette can include a constitutive promoter which expresses absent the need for an inducer. Constitutive promoters for use herein can include those providing a spectrum of expression level from highly expression constitutive promoters, to those providing more moderate and lower expression levels. In some embodiments, such as where two or more different expression cassettes are employed in the systems and methods herein, a first and a second type of expression cassette employ a different constitutive promoter. In some embodiments where two or more different expression cassettes are employed in the systems and methods herein, a first expression cassette employs an inducible promoter, and a second expression cassette employs a constitutive promoter.

In some cases, the sequence identity of the promoter sequences may be a sequence having at least 80%, 90%, 95%, 96%, 97%, 98%, 99% or 99.5% sequence identity to SEQ ID NOs: 1-8 set forth in Table No. 1. In some embodiments, the one or more promoters are selected from the group consisting of adh1+, alcohol dehydrogenase (ADH1, ADH2, ADH4), AHSB4m, AINV, alcohol oxidase I (AOX1), alcohol oxidase 2 (AOX2), dihydroxyacetone synthase (DAS), enolase (ENO, ENO1), formaldehyde dehydrogenase (FLD1), FMD, formate dehydrogenase (FMDH), G1, G6, GAA, GCW14, gdhA, glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, β-galactosidase (lac4), LEU2, melO, MET3, nmt1, NSP, pcbC, PETS, peroxin 8 (PEX8), phosphoglycerate kinase (PGK, PGK1), phol, PHO5, PH089, phosphatidylinositol synthase (PIS1), PYK1, pyruvate kinase (pki1), RPS7, sorbitol dehydrogenase (SDH), 3-phosphoserine aminotransferase (SERI), SSA4, TEF, translation elongation factor 1 alpha (TEF1), THI11, homoserine kinase (THR1), tpi, TPS1, triose phosphate isomerase (TPI1), XRP2, and YPT1.

### Terminators

An expression cassette can include a terminator 3′ to the protein coding sequence. In some embodiments the terminator and promoter sequences are from the same gene source (e.g. a DAS promoter and a DAS terminator). In other embodiments, the promoter and terminator of an expression cassette are derived from different gene sources. In some embodiments, such as where two or more different expression cassettes are employed in the systems and methods herein, each expression cassette can employ a different terminator sequence. In some embodiments, such as where two or more different expression cassettes are employed in the systems and methods herein, each expression cassette can employ the same terminator.

In some cases, the sequence identity of the terminator sequences may be a sequence having at least 80%, 90%, 95%, 96%, 97%, 98%, 99% or 99.5% sequence identity to SEQ ID NOs: 9-10 set forth in Table No. 2. In some embodiments, a terminator for an expression cassette is selected from the group consisting of adh1+, alcohol dehydrogenase (ADH1, ADH2, ADH4), AHSB4m, AINV, alcohol oxidase I (AOX1), alcohol oxidase 2 (AOX2), dihydroxyacetone synthase (DAS), enolase (ENO, ENO1), formaldehyde dehydrogenase (FLD1), FMD, formate dehydrogenase (FMDH), G1, G6, GAA, GCW14, gdhA, glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, β-galactosidase (lac4), LEU2, melO, MET3, nmt1, NSP, pcbC, PETS, peroxin 8 (PEX8), phosphoglycerate kinase (PGK, PGK1), phol, PHO5, PH089, phosphatidylinositol synthase (PIS1), PYK1, pyruvate kinase (pki1), RPS7, sorbitol dehydrogenase (SDH), 3-phosphoserine aminotransferase (SERI), SSA4, TEF, translation elongation factor 1 alpha (TEF1), THI11, homoserine kinase (THR1), tpi, TPS1, triose phosphate isomerase (TPI1), XRP2, and YPT1.

### Signal Secretion Sequences

In embodiments, the systems and methods provided herein are designed for secretion of a desired recombinant heterologous protein. In some cases, it is achieved by fusing a secretion signal in-frame to the coding region of the recombinant heterologous protein in the plurality of expression cassettes integrated into the host cell genome. In some embodiments, a plurality of the expression cassettes can include a heterologous secretion signal (e.g., not derived natively from the heterologous protein to be expressed). In some embodiments, a plurality of the expression cassettes employed in the systems and methods herein, can include a heterologous secretion signal and lack any naturally occurring secretion signal.

In some embodiments, such as where two or more different expression cassettes are employed in the systems and methods herein, each expression cassette can employ a different secretion signal peptide sequence. In some embodiments, such as where two or more different expression cassettes are employed in the systems and methods herein, each expression cassette can employ the same secretion signal peptide sequence. Exemplary secretion signals include but are not limited to the mating factor alpha-factor pro sequence from Saccharomyces cerevisiae, an Ost1 signal sequence, hybrid Ost1-alpha-factor pro sequence, and synthetic signal sequences.

In some cases, the sequence identity of the signal peptides may be a sequence having at least 80%, 90%, 95%, 96%, 97%, 98%, 99% or 99.5% sequence identity to SEQ ID NOs: 11-12 set forth in Table No. 3. In any one of the embodiments disclosed herein, the signal peptide may be selected from the group consisting of acid phosphatase, albumin, alkaline extracellular protease, α-mating factor, amylase, β-casein, carbohydrate binding module family 21-starch binding domain, carboxypeptidase Y, cellobiohydrolase I, dipeptidyl protease, glucoamylase, heat shock protein (e.g., bacterial Hsp70), hydrophobin, inulase, invertase, killer protein or killer toxin (e.g., 128 kDa pGKL killer protein, α-subunit of the K1 killer toxin (e.g., Kluyveromyces lactis, K1 toxin KILM1, K28 pre-pro-toxin, Pichia acaciae), leucine-rich artificial signal peptide CLY-L8, lysozyme, phytohemagglutinin, maltose binding protein, P-factor, Pichia pastoris Dse, Pichia pastoris Exg, Pichia pastoris Pir1, Pichia pastoris Scw, Pir4, and any combination thereof.

### Selectable Markers

In the systems and methods provided herein, an expression cassette for integration in the host cell can be designed lacking a selectable marker. In some other cases, an expression cassette for integration in the host cell can be designed for identification of a positive integrant using one or more selectable markers. In some cases, an expression cassette for integration in the host cell can include one or more antibiotic resistance genes, auxotrophic markers or a combination thereof. In some embodiments, such as where two or more different expression cassettes are employed in the systems and methods herein, each expression cassette can employ a different combination of selectable markers. In some embodiments, such as where two or more different expression cassettes are employed in the systems and methods herein, each expression cassette can employ the same combination of selectable markers. Exemplary selectable markers can include: an antibiotic resistance gene (e.g. zeocin, ampicillin, blasticidin, kanamycin, nurseothricin, chloroamphenicol, tetracycline, triclosan, ganciclovir) or any combination thereof. Other examples of selectable markers can include an auxotrophic marker (e.g. ade1, arg4, his4, ura3, met2) or any combination thereof. In some cases, the auxotrophic marker may be a defective auxtrophic marker, e.g., leu2-d or a variant of leu2-d involved in leucine metabolism (Betancur et. al, 2017). In some cases, the sequence identity of the selectable markers may be a sequence having at least 80%, 90%, 95%, 96%, 97%, 98%, 99% or 99.5% sequence identity to SEQ ID NOs: 17-25 set forth in Table No. 5.

### Helper Factor Proteins

An engineered host cell may comprise one or more copies of a helper factor gene encoding a helper factor protein. In some cases, the methods herein can include transformation with an expression cassette for the expression of a helper factor, such as one that promotes protein folding, protein stability, protein translation and/or that increase transcription from a promoter.

The expression cassettes comprising one or more helper factor genes may comprise promoters used in the expression cassettes used for the expression of the heterologous gene. Alternatively, an expression cassette comprising a helper factor gene may comprise a promoter different from any of the promoters integrated into the host genome used to express the heterologous gene.

Exemplary helper factors proteins include proteins such as Serine/threonine protein kinase 2 (Kin2), squalene synthase (ERG9), protein disulfide isomerase 1 (PDI1), heat shock proteins such as SSA1, SSA4, chaperone proteins such as SSB1, SSE1, BiP, transcriptional activators such as HAC1, ER Membrane Protein Complex Subunit 1 (EMC1), YNL181W oxidoreductase, integral membrane protein zinc metalloprotease Ste24, 14-3-3 protein Bmh2, ER oxidoreductin 1 (Ero1). In some cases, the sequence identity of the helper factor proteins may be a sequence having at least 80%, 90%, 95%, 96%, 97%, 98%, 99% or 99.5% sequence identity to SEQ ID NOs: 26-39 set forth in Table No. 6.

### Exemplary Combinations of Genetic Elements for Expression Cassettes

The genetic elements of the expression cassette can be designed to be suitable for expression in the intended host cell organism. For example, the genetic elements in the plurality of expression cassettes can be codon-optimized for effective expression in the intended host cell organism.

An expression cassette can be constructed to comprise any combination of the genetic elements (e.g., promoters, terminators, signal sequence, selectable markers, transgene coding sequence etc.) In some cases, a host strain for the expression of the OVD coding sequence may be generated by transforming an expression cassette containing the pAOX1 promoter, the alpha mating factor secretion signal along, a tAOX1 terminator along with a Ura3 selection marker. In some cases, the pDAS2 promoter may be combined with the alpha mating factor secretion signal and a tAOX1 terminator (no selectable marker) to generate a cassette for the expression of the OVD coding sequence. In some cases, an expression cassette can include the pPEX11 promoter and a tAOX1 terminator. In some cases, an expression cassette can include the pPEX11 promoter driving a helper factor protein such as HAC1 with a tAOX1 terminator. In some cases, an expression cassette for the expression of OVD may include the pAOX1 promoter, an alpha mating factor secretion signal and a tAOX1 terminator along with a selection marker.

In some cases, a host strain for the expression of the OVD coding sequence may be generated by transforming an expression cassette containing a pAOX1 promoter, an alpha mating factor secretion signal, a tAOX1 terminator along with a selection marker. In some cases, a host strain for the expression of the OVD coding sequence may be generated by transforming an expression cassette containing a pAOX1 promoter, an alpha mating factor secretion signal, a tAOX1 terminator along with a selection marker. In some cases, a host strain for the expression of the OVD coding sequence may be generated by transforming an expression cassette containing a pDAS2 promoter, an alpha mating factor secretion signal, a tAOX1 terminator along with a selection marker. In some cases, a host strain for the expression of the OVD coding sequence may be generated by transforming an expression cassette containing a pFLD1 promoter, an alpha mating factor secretion signal, a tAOX1 terminator along with a selection marker.

In some cases, a host strain for the expression of the PGA coding sequence may be generated by transforming an expression cassette containing a pAOX1 promoter, an alpha mating factor secretion signal, a tAOX1 terminator along with a selection marker. In some cases, a host strain for the expression of the PGA coding sequence may be generated by transforming an expression cassette containing a pFDH1 promoter, an alpha mating factor secretion signal, a tAOX1 terminator along with a selection marker. In some cases, a host strain for the expression of the PGA coding sequence may be generated by transforming an expression cassette containing a pFLD1 promoter, an alpha mating factor secretion signal, a tAOX1 terminator along with a selection marker.

### Methods for Co-Transformation of Expression Cassettes

The methods herein employ co-transformation to generate the multiple expression cassettes into the genome. The expression cassettes (e.g., 1, 2, 3 or more different cassettes) as DNA are mixed together and transformed into a host cell. Alternate methods may employ pre-joined cassettes, whereby the DNA sequence for the multiple copies of a single cassette, or the DNA sequence for different expression cassettes are linked in vitro (e.g., in a single plasmid) prior to transformation. In some cases, one or more plasmids comprising a designed copy number of the heterologous protein (e.g., recombinant ovalbumin) may be linearized and combined in a starting mixture of nucleic acids for a single transformation reaction into the host cell (e.g., Pichia). For example, plasmid 1 can contain 2 head-to-tail copies of a cassette with a pAOX1 promoter, an alpha mating factor secretion signal fused in frame with a ovalbumin (OVA) cDNA, followed by a tAOX2 terminator, while plasmid 2 can be constructed with four head to tail copies of a cassette containing a pFLD1 promoter, an alpha mating factor secretion signal fused in frame with a PGA cDNA, followed by a tAOX1 terminator. Both plasmids may include a loxZeo selection cassette. In a combinatorial transformation, plasmids 1 and 2 are both linearized and combined in a starting mixture of nucleic acids for a single transformation reaction into Pichia and a transformation strain A is recovered.

In other cases, one or more plasmids comprising one or more copies of the expression cassettes may be sequentially transformed into the host cell. For example, strain A obtained from combinatorial transformation previously can be used as the starting material. Strain A can then be sequentially transformed with two plasmids (plasmid 3 and 4), each containing a PGK1 signal sequence fused in frame to an ovalbumin protein encoding cDNA. Each plasmid can contain a unique combination of promoter and terminator. For example, plasmid 3 may contain pDAS2 and tAOX2 while plasmid 4 may contain pFLD1 with tAOX1. The backbone in plasmid 3 could include a LoxZeo resistance gene. The backbone in plasmid 4 could include a Hygromycin resistance. First strain A is transformed with plasmid 3 and a transformant strain B is recovered by selection. Then strain B is transformed with plasmid 4 and the final transformant strain C is recovered by selection. In some cases, the plasmids may be integrated in the same genomic locus, or in the vicinity of the same genomic locus. In other cases, the plasmids may be integrated in different genomic loci.

In some cases, the selectable markers introduced into the backbone of the expression cassette may be excised between the sequential transformations, described elsewhere in this disclosure. In some cases, the sequential transformations may be performed with plasmids bearing different selectable markers.

Integration of Expression Cassettes into Host Cell Genome

In some embodiments, multiple expression cassettes are integrated into a single site in the genome of the host cell, such as methylotrophic yeast cell, e.g., a Pichia cell. In some embodiments, multiple expression cassettes are integrated within the vicinity of one another site in the genome of the host cell, such as methylotrophic yeast cell, e.g., a Pichia cell. In some cases, where two or more different expression cassettes are employed in the systems and methods herein, the integration sites of the first and the second expression cassette can be located on the same chromosome. In some cases, additionally, a third expression cassette can be integrated in the genome of the engineered cell at an integration site different from that of the first cassette and second cassette. In some cases, additionally, a third expression cassette can be integrated in the genome of the engineered cell at the same integration site as that of the first cassette and second cassette. In some cases, where two or more different expression cassettes are employed in the systems and methods herein, the integration sites of the plurality of the expression cassettes can be located on homologous sites in different chromosomes of the host cell genome.

In some embodiments, the multiple expression cassettes are integrated in tandem at a genomic site of the host cell, where all the cassettes are in a single orientation (e.g., with reference to 5′ to 3′ orientation of the cassette). In some embodiments, the multiple expression cassettes are integrated into the genome of the host cell, such as methylotrophic yeast cell, e.g., a Pichia pastoris cell, in arrangements where one or more of the cassettes is in a different orientation as compared to other cassettes. In some cases, where two or more different expression cassettes are employed in the systems and methods herein, a first and a second expression cassette are integrated into the genome in an opposite 5′ to 3′ orientation. In some cases, where two or more different expression cassettes are employed in the systems and methods herein, a first and a second expression cassette are integrated into the genome in the same 5′ to 3′ orientation. In some cases, additionally, a third expression cassette can integrate in the genome of the engineered cell at an integration site in a 5′ to 3′ orientation different from that of the first cassette, the second cassette, or both the first and the second cassette. In some cases, additionally, a third expression cassette can be integrated in the genome of the engineered cell in the same 5′ to 3′ orientation as that of the first cassette, the second cassette or both the first and the second cassette.

In some embodiments, multiple expression cassettes may be ectopically integrated by non-homologous recombination in a single genomic locus of the host cell genome. In some embodiments, multiple expression cassettes may be ectopically integrated by non-homologous recombination in the vicinity of or at the same genomic locus in the host cell genome. In some embodiments, multiple expression cassettes may be ectopically integrated by non-homologous recombination on the same chromosome in the host cell genome. In some embodiments, multiple expression cassettes may be ectopically integrated by non-homologous recombination on different chromosomes in the host cell genome.

In some cases, multiple expression cassettes can be integrated into the genome of the host cell, e.g., Pichia cell, by non-homologous recombination methods. In some cases, where two or more different expression cassettes are employed in the systems and methods herein, each expression cassette of the plurality of the expression cassettes can be integrated by non-homologous recombination. In some cases, where two or more different expression cassettes are employed in the systems and methods herein, at least one of the expression cassettes in the plurality of expression cassettes can be integrated by non-homologous recombination.

In some cases, where two different expression cassettes are employed in the systems and methods herein, the first and the second expression cassettes can be both integrated by non-homologous recombination. In some cases, multiple expression cassettes can be integrated into the host cell genome by homologous recombination. In some cases, where two different expression cassettes are employed in the systems and methods herein, the first expression cassette can be integrated by non-homologous recombination and the second expression cassette can be integrated by homologous recombination. In some cases, additionally, a third expression cassette can integrate in the genome of the engineered cell by a recombination method different from that of the first cassette, the second cassette or both the first and the second cassette. In some cases, additionally, a third expression cassette can be integrated in the genome of the engineered cell by the same recombination method as the first cassette, the second cassette or both the first and the second expression cassette.

In some cases, there is insubstantial sequence homology between a sequence in an expression cassette and a corresponding sequence in the host cell genome. For instance, where two or more different expression cassettes are employed in the systems and methods herein, the genomic locus of integration in the host cell does not share sequence homology with a first promoter, second promoter, first gene, second gene, first signal sequence, second signal sequence, first selective marker or second selective marker.

In some cases, there is sequence homology between a sequence in the host cell genome and one or more sequences with an expression cassette. In some cases, the sequence homology resides at or in part at a sequence at the 5′ and 3′ ends of a linearized expression cassette. In some cases, where two different expression cassettes are employed in the systems and methods herein, and where the first expression cassette and second expression cassette are linear molecules, the first expression cassette or the second expression cassette can comprise homology at the 5′ end with the host cell genome locus. For instance, the sequence homology between a sequence in the genomic locus of integration and a sequence at the 5′ sequence or the 3′ sequences of an expression cassette may be at least 5 bp, at least 10 bp, at least 20 bp, at least 30 bp, at least 40 bp, at least 60 bp, at least 80 bp, at least 100 bp, at least 120 bp, at least 150 bp, at least 180 bp, at least 200 bp, at least 250 bp, at least 300 bp, at least 350 bp, at least 400 bp, at least 450 bp, at least 500 bp, at least 600 bp, at least 700 bp long, at least 800 bp long, at least 900 bp long or at least 1000 bp long. In some cases, the sequence homology between a sequence in the genomic locus of integration and a sequence at the 5′ sequence or the 3′ sequences of an expression cassette may be at most 10 bp, at most 20 bp, at most 30 bp, at most 40 bp, at most 60 bp, at most 80 bp, at most 100 bp, at most 120 bp, at most 150 bp, at most 180 bp, at most 200 bp, at most 250 bp, at most 300 bp, at most 350 bp, at most 400 bp, at most 450 bp, at most 500 bp, at most 600 bp, at most 700 bp long, at most 800 bp long, at most 900 bp long or at most 1000 bp long.

In some cases, the expression cassettes may be integrated by homologous recombination by relying on the sequence homology between a sequence in the expression cassette and a corresponding sequence in the host cell genome. In some cases, the homologous recombination may rely on the sequence homology between a promoter sequence in a first expression cassette and the genomic promoter sequence. For instance, the homologous recombination may rely on the sequence homology between an AOX1 promoter in the expression cassette and the genomic AOX1 sequence. In some cases, the homologous recombination may rely on the sequence homology between a secretion signal sequence in a first expression cassette and the secretion signal sequence in the host cell genome cell. In some cases, the homologous recombination may rely on the sequence homology between a selective marker sequence in a first expression cassette and the genomic sequence. For instance, the homologous recombination may rely on the sequence homology between a URA3 selective marker in the expression cassette and the genomic URA3 sequence.

### Excision of Selectable Markers and Transformant Screening

In the methods provided herein, expression cassettes containing genetic information are inserted into host cells. In some embodiments, clonal populations of successful transformants may be isolated by any means known in the art. In some cases, the use of a increasing concentrations of antibiotics, such as Geneticin® (G418) and Zeocin™, and their corresponding antibiotic resistance genes can be used for screening for multi-copy integrations. Individual colonies can be picked and verified for the integration of expression cassettes into the host cell genome by standard molecular biological methods that are known to one trained in the art (i.e. colony PCR, genomic sequencing). Individual protein expression can be determined by standard molecular biology methods (e.g. Western blot, SDS-PAGE with known standard protein).

In some embodiments, the methods employed herein comprise integration of an expression cassettes comprising a selectable marker, followed by excision of the selectable marker using a site-specific genome editing system. In some cases, a selectable marker sequence in the expression cassette, for example, an antibiotic resistance gene or an auxotrophic marker can be bordered by a pair of lox sites (e.g., lox71 and lox66; exemplary sequences for which are provided in Table 5; SEQ ID NO: 23 or 24). Cre recombinase expression in the engineered cell can be used to excise the selectable marker gene from the loxP sites. In some cases, (using sequences such as exemplified in Table 5, SEQ ID NO: 22) the Cre recombinase and the selectable marker sequence can be combined in an expression cassette. In some cases, the expression cassette can additionally contain a Cre gene intron sequence to protect against leakage in the expression of the Cre protein. In some cases, the Cre recombinase may be expressed separately using an episomal plasmid. In some cases, other recombinase systems such as a FLP/FRT site-specific recombination system may be used for excision of the selectable markers after integration into the genome. In some embodiments, excision of sequences between the lox (or other recombinase sites), also excises additional sequences such as vector backbone, bacterial origin of replication, bacterial selectable marker and other sequences (some examples of which are provided in Table 5). In some embodiments, the recombinase expression cassette is included in the sequences excised by expression of the recombinase in the host cell.

A variety of methods are available to identify those cells having an altered genome without the use of a selectable marker. In some embodiments, such methods include but are not limited to PCR methods (including quantitative PCR), sequencing methods, nuclease digestion, e.g., restriction mapping, Southern blots, and any combination thereof. Phenotypic readouts, for example, a predicted gain or loss of function, can also be used as a proxy for effecting the intended genomic modification(s).

Using the methods provided herein, marker-less recovery (including loss of the vector backbone and other excised sequences) of a transformed cell comprising a successfully integrated expression cassette at a single locus can occur within a frequency of at least 10%, 20%, 30%, 50%, 60%, 70%, 80%, 90%, or 100% of contacted host cells or clonal population thereof, screened. In certain embodiments, marker-less recovery of a transformed cell comprising successfully integrated expression at two, three, four, or five loci can occur at least 10%, 20%, 30%, 40%, 50%, 60%, 70%, 80%, 90%, or 100% of contacted host cells or clonal population thereof, screened.

In some cases, a first linear expression cassette can recombine with a second linear expression cassette in the host cell to form two or more different, circular, extrachromosomal nucleic acids in the host cell. For example, the host cell can be contacted with two or more second linearized plasmids comprising one or more copies of the first or the second expression cassette, the first and second linear expression cassettes undergo homologous recombination to form a circular, episomal or extrachromosomal nucleic acid comprising the coding sequence for the selectable marker. Once circularized, the extrachromosomal nucleic acid includes a coding sequence for a selectable marker, and suitable regulatory sequences such as a promoter and/or a terminator that enables expression of the marker in the host cell.

In some embodiments, the methods described herein can further comprise the step of eliminating the circularized extrachromosomal vector from the host cell, for example, once a selected host cell has been identified as comprising the desired genomic integration(s). In some embodiments, elimination of a plasmid encoding the selective marker from a selected cell can be achieved by allowing the selected cells to undergo sufficient mitotic divisions such that the plasmid is effectively diluted from the population. Alternatively, plasmid-free cells can be selected by selecting for the absence of the plasmid, e.g., by selecting against a counter-selectable marker (such as, for example, URA3) or by plating identical colonies on both selective media and non-selective media and then selecting a colony that does not grow on the selective media but does grow on the nonselective media.

### Host Cell Engineering

A host cell can be modified in addition to and separately from integrating the expression cassettes. Such modification can be performed prior to or subsequent to transformation with the expression cassettes. In some instances, the modification contributes to the growth features and/or expression features of the host cell and thereby assists in the production of high protein tiers under fermentation conditions.

In some embodiments, the modification alters the host cell response to an inducer. For example, one such modification may be a modification which alters the growth characteristics of the host cell (e.g., Pichia) to methanol. In some embodiments, a mutated host is used as the host cell for further transformation and integration of expression cassettes where one or more of the cassettes includes a promoter inducible by methanol. In some embodiments, the modification includes the expression of one or more factors that increase the amount of, accumulation of or the production of an active form of the protein encoded by the expression cassettes. Such modifications can include the expression of one or more helper factors (such as transcription factors, chaperones and other proteins that participate in protein folding), post-transcriptional modification enzymes (e.g., phosphorylases, phosphatases, glycosylation and deglycosylation enzymes).

In some embodiments, a host cell (e.g., a Pichia cell) may be engineered to display increased non-homologous recombination (NHEJ) as compared to homologous recombination. For instance, in some cases, a host cell (e.g., a Pichia cell) may be engineered to overexpress a gene that is involved in non-homologous recombination activity of the cell (i.e., one or more genes that encode proteins that drives the NHEJ pathway or contribute to NHEJ). Examples of NHEJ pathway genes for Pichia include, but are not limited to, YKU70, YKU 80, DNL4, Rad50, Rad 27, MRE11, and POL4. The names of genes may be different for different host cells. The increase in NHEJ activity can be a reduction in homologous recombination of at least 20%, 30%, 40%, 50%, 60%, 70%, 80%, 90%, 100% or any percent reduction in between these percentages, as compared to a host cell that does not overexpress a gene controlling NHEJ in the cell, for example, the YKU70 gene locus of a Pichia cell.

To alleviate the depletion of intracellular amino acids concentrations that may occur due to high recombinant protein production, the host cells may be engineered to improve the supply of amino acids and therefore protein production in P. pastoris. In some embodiments, overexpression of GCN4 (encoding a general transcriptional activator of amino acid biosynthesis, direct overexpression of metabolic enzymes in the anabolism of serine, isoleucine, alanine and aromatic amino acids, or of a fungal carboxylesterase can be used to optimize the synthesis pathways of amino acids by tuning enzyme abundance or their kinetics.

To overcome the constraints of energetic inefficiencies that may occur due to high recombinant protein production, the host cells can be optimized for an improved supply of precursors involved in cellular redox and energy efficiency. In some embodiments, strategies may include deletion of genes diverting carbon towards fermentative pathways, overexpression of malate dehydrogenase, which could increase the supply of mitochondrial NADH, or overexpression of enzymes in the oxidative part of the PPP (e.g., NADH oxidase) causing an increased supply of NADPH and precursors and thereby higher titer protein production.

Undesired proteolysis of heterologous proteins expressed in P. pastoris does not only lower the product yield or biological activity but can also complicate downstream processing of the intact product as the degradation products will have similar physicochemical and affinity properties. In order to alleviate the proteolysis problem, protease-deficient host cells strains lacking proteases can be used. Examples of proteases include PEP4, carboxypeptidase Y (PRC1) and proteinase B (PRB1). Examples of such P. astoris protease-deficient strains include SMD1163 (Δhis4 Δpep4 Δprb1), SMD1165 (Δhis4 Δprb1) and SMD1168 (Δhis4 Δpep4).

High recombinant protein production can induce secretory bottlenecks in the form of inappropriate mRNA structure, incomplete protein folding or protein translocation to the ER. Host cells can be engineered to overcome potential secretory bottleneck by the overexpression of folding helper proteins such as iP/Kar2p, DnaJ, PDI, PPIs and Ero1p or, alternatively, overexpression of HAC1, a transcriptional regulator of the UPR pathway genes.

In some cases, heterologous protein production in host cells may be accompanied by high mannose glycan structures, affecting serum half-life or triggering of allergic reactions in the human body. To alleviate this problem, the host cells may be further engineered to include the knockout of protein-O-mannosyltransferases (PMTs) or the yeast Golgi protein α-1,6-mannosyltransferase encoded by OCH1. In other cases, the host cells may be engineered to express a Trichoderma reesei α-1,2-mannosidase or one of several glycosyltransferases and glycosidases (e.g., β-1,2-N-acetylglucosaminyl-transferase 1, uridine 5′-diphosphate (UDP)-GlcNAc transporter, mouse mannosidase MnsIA catalytic domain fused to the N-terminal localization peptide of the ER protein Sec12 from S. cerevisiae, human GlcNAc transferase GnTI fused to the leader sequence from the S. cerevisiae Golgi protein Mnn9, overexpression of Drosophila melanogaster mannosidase II (ManII) or rat GlcNAc transferase GnTII, overexpression of Schizosaccharomyces pombe galactose epimerase or human β-1,4 galactosyl transferase) carrying proper targeting signals may be used. In some cases, genes involved in sialic acid synthesis, transport and transfer may be co-expressed, for example, human UDP-N-acetylglucosamine-2-epimerase/N-acetylmannosamine kinase (GNE), human N-acetylneuraminate-9-phosphate synthase (SPS), human CMP-sialic acid synthase (CSS), mouse CMP-sialic acid transporter (CST), to achieve optimal sialyated N-glycans.

In some cases, the recombinant host cell may be a methanotroph. Among methanotrophs, Komagataella pastoris and Komagataella phaffii are preferable (also known as Pichia pastoris). Examples of strains in the Pichia genus include Pichia pastoris strains. Examples can include NRRL Y-11430, BG08, BG10, NRRL Y-11430 GS115 (NRRL Y-15851), GS190 (NRRL Y-18014), PPF1 (NRRL Y 18017), PPY1200H, YGC4, and strains derived therefrom. Other examples of P. pastoris strains that may be used as host cells include but are not limited to CBS7435 (NRRL Y-11430), CBS704 (DSMZ 70382) or derivatives thereof. Other examples of methanol-utilizing yeast include yeasts belonging to Ogataea (Ogataea morpha), Candida (Candida boidinii), Torulopsis (Torulopsis) or Komagataella.

Further examples of suitable host cell organisms include but are not limited to: Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, Trichoderma vireus, Aspergillus oryzae, Bacillus subtilis, Escherichia coli, Myceliophthora thermophila, Neurospora crassa, Pichia pastoris, Pichia Pastoris “MutS” strain (Graz University of Technology (CBS7435MutS) or Biogrammatics (BG11)), Komagatella phaffi, and Komagatella pastoris.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “at least” in the phrase “a ratio of the helper factor encoding sequence to the heterologous protein encoding sequence is at least 1:10”, as an example, means that a covered ratio may have more copies of the helper factor encoding sequence relative to the number of copies of the heterologous protein encoding sequence. In other words, a condition of “at least 1:10”, means that there must be “at least 1” copy of the helper factor encoding sequence per 10 copies of the heterologous protein encoding sequences, e.g., there can be 1 copy, 2 copies, 3 copies, 4 copies or more copies. On the other hand, the term “at most” in the phrase “a ratio of the helper factor encoding sequence to the heterologous protein encoding sequence is at most 1:2”, for example, means that a covered ratio may have fewer copies of the helper factor encoding sequence relative to the number of copies of the heterologous protein encoding sequence. In other words, a condition of “at most 1:2”, means that there must be “at most 1” copy of the helper factor encoding sequence per 2 copies of the heterologous protein encoding sequences. Note that the ratio of 1:2 is equivalent the ratio of 5:10; thus, the equivalent term of “at most 5:10”, would cover 5 copies of the helper factor sequence per 10 copies of the heterologous protein encoding sequence, 4 copies of the helper factor sequence per 10 copies of the heterologous protein encoding sequence, 3 copies of the helper factor sequence per 10 copies of the heterologous protein encoding sequence, and so forth.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Herein the term “sequence identity”, such as for the purpose of assessing percent complementarity, may be measured by any suitable alignment algorithm. In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. Herein percentage sequence identity can refer to sequences and their alignment over the span of a query sequence. If one sequence is shorter than the other, than the percentage identity can be considered over the span of the shorter sequence. Herein “percentage coverage” can refer to the number of nucleotides or amino acids that align identically with the longer of the two sequences as a percentage of the number of nucleotides or amino acids in the longer sequence.

Herein one polynucleotide is referred to another polynucleotide as being a “copy” of the other if it has 100% sequence identity to another polynucleotide and is the same length. In some cases, one polynucleotide is referred to another polynucleotide as being a “copy” of the other if it has a different sequence, but the protein encoded by the two polynucleotides has the same amino acid sequence. Herein a polynucleotide is “different” from a set of polynucleotides if it is not a copy of any element of the set, or for all those elements of a sets that it is a copy, it contains chemical differences apart from its genetic or amino acid sequence that distinguishes it from that element.

Herein an “expression cassette” is any polynucleotide that contains a subsequence that codes for a transgene and can confer expression of that subsequence when contained in a host cell and is heterologous to that host organism.

The section headings used herein are for organizational purposes only and are not to be construed as limiting the subject matter described.

TABLE 1
Promoter sequences

| SEQ | | ANNO- |
|---|---|---|
| ID | | TA- |
| NO. | SEQUENCE | TION |
| 1 | AACATCCAAAGACGAAAGGTTGAATGAAACCTTTTTGCCATCCGACATCCACAGGTCCATTCTCACACATAAGTGCCAAACGCAACAGG | pAOX1 |
| | AGGGGATACACTAGCAGCAGACCGTTGCAAACGCAGGACCTCCACTCCTCTTCTCCTCAACACCCACTTTTGCCATCGAAAAACCAGCC | |
| | CAGTTATTGGGCTTGATTGGAGCTCGCTCATTCCAATTCCTTCTATTAGGCTACTAACACCATGACTTTATTAGCCTGTCTATCCTGGCCC | |
| | CCCTGGCGAGGTTCATGTTTGTTTATTTCCGAATGCAACAAGCTCCGCATTACACCCGAACATCACTCCAGATGAGGGCTTTCTGAGTGT | |
| | GGGGTCAAATAGTTTCATGTTCCCCAAATGGCCCAAAACTGACAGTTTAAACGCTGTCTTGGAACCTAATATGACAAAAGCGTGATCTC | |
| | ATCCAAGATGAACTAAGTTTGGTTCGTTGAAATGCTAACGGCCAGTTGGTCAAAAAGAAACTTCCAAAAGTCGGCATACCGTTTGTCTT | |
| | GTTTGGTATTGATTGACGAATGCTCAAAAATAATCTCATTAATGCTTAGCGCAGTCTCTCTATCGCTTCTGAACCCCGGTGCACCTGTGC | |
| | CGAAACGCAAATGGGGAAACACCCGCTTTTTGGATGATTATGCATTGTCTCCACATTGTATGCTTCCAAGATTCTGGTGGGAATACTGC | |
| | TGATAGCCTAACGTTCATGATCAAAATTTAACTGTTCTAACCCCTACTTGACAGCAATATATAAACAGAAGGAAGCTGCCCTGTCTTAAA | |
| | CCTTTTTTTTTATCATCATTATTAGCTTACTTTCATAATTGCGACTGGTTCCAATTGACAAGCTTTTGATTTTAACGACTTTTAACGACAAC | |
| | TTGAGAAGATCAAAAAACAACTAATTATTGAA | |
| 2 | AAATCTGAGACAACGATGAACCTCCCATGTAGATTCCACCGCCCCAGTTACTTTTTTGGGCAATCCTGTTGATAAGATCCATTTTAGAGT | pDAS2 |
| | TGTTTCATGAAAGGATTACAGGCGTTGAAGGGTCAGAGAGATGCCAGAGAACAGACCAATTGGTAGTTTGCTAAAGTGGACGTCTGG | (1) |
| | CAGGTGCTCTATCGTGTTCTTTATTTAGGGCGTTACACTTAGTAGGATTACGTAACAATTTGGCTTAACCTTCTAAGTTAGAAAGAAACC | |
| | AAGAGGGGTCCTCTTTAACGTTCAGCAGTATCTAAAACACAAAACCTGCCCTCATAATACATCATTCTATCTGTCAAGCTGTGCTACCCC | |
| | ACAGAAATACCCCCAAGAGTTAAAGTGAAAAGAAAAGCTAAATCTGTTAGACTTCACCCCATAACAAACTTGATAGTTCCTGTAGCCAA | |
| | TGAAAGTTAACCCCATTCAATGTTCCGAGATCTAGTATGCTTGCTCCTATAAGGAACGAAGGGTTCCAGCTTCCTTACCCCATCAATGGA | |
| | AATCTCCTATTTACCCCCCACTGGAAAGATCCGTCCGAACGAACGGATAATAGAAAAAAGAAATTCGGACAAAATAGAACACTTATTTA | |
| | GCCAATGAAATCCATTTCCAGCATCTCCTTCAACTGCCGTTCCATCCCCTTTGTTGAGCTACACCATCGTCAGCCAGTACCGAATAGGAA | |
| | ACTTAACCGATATCTTGGAGAATTCTAATGCGCGAATGAGTTTAGCCTAGATATCCTTAGTGAAGGGTTGTTCCGATACTTCTCCACATT | |
| | CAGTCATTTCAGATGGGCAGCATTGTTATCATGAAGAAACGGAAACGGGCAGTAAGGGTTAACCGCCAAATTATATAAAGACAACATG | |
| | TCCCCAGTTTAAAGTTTTTCTTTCCTATTCTTGTATCCTGAGTGACCGTTGTGTTTAAAATAACAAGTTCGTTTTAACTTAAGACCAAAACC | |
| | AGTTACAACAAATTATTCCCCAACTAAACACTAAAGTTCACTCTTATCAAACTATCAAACATCAAA | |
| 3 | GATCTCTGAGACAACGATGAACCTCCCATGTAGATTCCACCGCCCCAATTACTGTTTTGGGCAATCCTGTTGATAAGACGCATTCTAGAG | pDAS2 |
| | TTGTTTCATGAAAGGGTTACGGGTGTTGATTGGTTTGAGATATGCCAGAGGACAGATCAATCTGTGGTTTGCTAAACTGGAAGTCTGG | (2) |
| | TAAGGACTCTAGCAAGTCCGTTACTCAAAAAGTCATACCAAGTAAGATTACGTAACACCTGGGCATGACTTTCTAAGTTAGCAAGTCAC | |
| | CAAGAGGGTCCTATTTAACGTTTGGCGGTATCTGAAACACAAGACTTGCCTATCCCATAGTACATCATATTACCTGTCAAGCTATGCTAC | |
| | CCCACAGAAATACCCCAAAAGTTGAAGTGAAAAAATGAAAATTACTGGTAACTTCACCCCATAACAAACTTAATAATTTCTGTAGCCAA | |
| | TGAAAGTAAACCCCATTCAATGTTCCGAGATTTAGTATACTTGCCCCTATAAGAAACGAAGGATTTCAGCTTCCTTACCCCATGAACAGA | |
| | AATCTTCCATTTACCCCCCACTGGAGAGATCCGCCCAAACGAACAGATAATAGAAAAAAGAAATTCGGACAAATAGAACACTTTCTCAG | |
| | CCAATTAAAGTCATTCCATGCACTCCCTTTAGCTGCCGTTCCATCCCTTTGTTGAGCAACACCATCGTTAGCCAGTACGAAAGAGGAAAC | |
| | TTAACCGATACCTTGGAGAAATCTAAGGCGCGAATGAGTTTAGCCTAGATATCCTTAGTGAAGGGTTGTTCCGATACTTCTCCACATTCA | |
| | GTCATAGATGGGCAGCTTTGTTATCATGAAGAGACGGAAACGGGCATTAAGGGTTAACCGCCAAATTATATAAAGACAACATGTCCCC | |
| | AGTTTAAAGTTTTTCTTTCCTATTCTTGTATCCTGAGTGACCGTTGTGTTTAATATAACAAGTTCGTTTTAACTTAAGACCAAAACCAGTT | |
| | ACAACAAATTATAACCCCTCTAAACACTAAAGTTCACTCTTATCAAACTATCAAACATCAAAGG | |
| 4 | TGCTCCTATAAGGAACGAAGGGTTCCAGCTTCCTTACCCCATCAATGGAAATCTCCTATTTACCCCCCACTGGAAAGATCCGTCCGAACG | pDAS2 |
| | AACGGATAATAGAAAAAAGAAATTCGGACAAAATAGAACACTTATTTAGCCAATGAAATCCATTTCCAGCATCTCCTTCAACTGCCGTT | (3) |
| | CCATCCCCTTTGTTGAGCTACACCATCGTCAGCCAGTACCGAATAGGAAACTTAACCGATATCTTGGAGACTTCTAATGCGCGAATGAGT | |
| | TTAGCCTAGATATCCTTAGTGAAGGGTTGTTCCGATACTTCTCCACATTCAGTCATTTCAGATGGGCAGCATTGTTATCATGAAGAAACG | |
| | GAAACGGGCAGTAAGGGTTAACCGCCAAATTATATAAAGACAACATGTCCCCAGTTTAAAGTTTTTCTTTCCTATTCTTGTATCCTGAGT | |
| | GACCGTTGTGTTTAAAATAACAAGTTCGTTTTAACTTAAGACCAAAACCAGTTACAACAAATTATTCCCCAACTAAACACTAAAGTTCAC | |
| | TCTTATCAAACTATCAAACATCAAA | |
| 5 | CTTCCCCATTTCACTGACAGTTTGTAGAAATAGGGCAACAATTGATGCAAATCGATTTTCAACGCATTGGTTTTGATAGCATTGATGATC | pPEX11 |
| | TTGGAGCTGTAAAAGTCCGGCTGGATAAGCTCAATGAAATAGGTTGGTTGATCTGGATCTTCTTTTGGGTCATTTTGTTCGCTCTGTATT | |
| | TCACAAATTGCCAGAATCTCTGCCAACCACAGTGGTAGGTCCAACTTGGTGTTCTGAATCACAGGCTTCCCCGGGTTGTTCTCTAAATAA | |
| | CCGAGGCCCGGCACAGAAATCGTAAACCGACACGGTATCTTTTGTCCGTCCGCCAGTATCTCATCAAGGTCGTAGTAGCCCATGATGAG | |
| | TATCAAAGGGGATTTGGTTATGCGATGCAACGAGAGATTGTTTATCCCAGATGCTGATGTAAAAACCTTAACCAGCGTGACAGTAGAA | |
| | ATAAGACACGTTAAAATTACCCGCGCTTCCCTAACAATTGGCTCTGCCTTTCGGCAAGTTTCTAACTGCCCTCCCCTCTCACATGCACCAC | |
| | GAACTTACCGTTCGCTCCTAGCAGAACCACCCCAAAGTTTAATCAGGACCGCATTTTAGCCTATTGCTGTAGAACCCCACAACATAACCT | |
| | GGTCCAGAGCCAGCCCTTTATATATGGTAAATCCCGTTTGAACTTCGAAGTGGAATCGGAATTTTTACATCAAAGAAACTGATACTGAA | |
| | ACTTTTGGCTTCGACTTGGACTTTCTCTTAATC | |
| 6 | AAATAAATGGCAGAAGGATCAGCCTGGACGAAGCAACCAGTTCCAACTGCTAAGTAAAGAAGATGCTAGACGAAGGAGACTTCAGAG | pFDH1 |
| | GTGAAAAGTTTGCAAGAAGAGAGCTGCGGGAAATAAATTTTCAATTTAAGGACTTGAGTGCGTCCATATTCGTGTACGTGTCCAACTGT | |
| | TTTCCATTACCTAAGAAAAACATAAAGATTAAAAAGATAAACCCAATCGGGAAACTTTAGCGTGCCGTTTCGGATTCCGAAAAACTTTT | |
| | GGAGCGCCAGATGACTATGGAAAGAGGAGTGTACCAAAATGGCAAGTCGGGGGCTACTCACCGGATAGCCAATACATTCTCTAGGAA | |
| | CCAGGGATGAATCCAGGTTTTTGTTGTCACGGTAGGTCAAGCATTCACTTCTTAGGAATATCTCGTTGAAAGCTACTTGAAATCCCATTG | |
| | GGTGCGGAACCAGCTTCTAATTAAATAGTTCGATGATGTTCTCTAAGTGGGACTCTACGGCTCAAACTTCTACACAGCATCATCTTAGTA | |
| | GTCCCTTCCCAAAACACCATTCTAGGTTTCGGAACGTAACGAAACAATGTTCCTCTCTTCACATTGGGCCGTTACTCTAGCCTTCCGAAG | |
| | AACCAATAAAAGGGACCGGCTGAAACGGGTGTGGAAACTCCTGTCCAGTTTATGGCAAAGGCTACAGAAATCCCAATCTTGTCGGGAT | |
| | GTTGCTCCTCCCAAACGCCATATTGTACTGCAGTTGGTGCGCATTTTAGGGAAAATTTACCCCAGATGTCCTGATTTTCGAGGGCTACCC | |
| | CCAACTCCCTGTGCTTATACTTAGTCTAATTCTATTCAGTGTGCTGACCTACACGTAATGATGTCGTAACCCAGTTAAATGGCCGAAAAA | |
| | CTATTTAAGTAAGTTTATTTCTCCTCCAGATGAGACTCTCCTTCTTTTCTCCGCTAGTTATCAAACTATAAACCTATTTTACCTCAAATACCT | |
| | CCAACATCACCCACTTAAACAG | |
| 7 | CAGCCATTAATCTCACCTCAGTTTTTGAATCAGTAGAATTTTTAATGAAACAAACGGTTGGTATATTATTTGATAGAGTTGCCAAATTTCC | pFLD1 |
| | AAAGATAAATTTTTCATCAGGTAATATCCTGAATACCGTAACATAGTGACTATTGGAAGACACTGCTATCATATTATATTTCGGATAAAA | |
| | ATCCAAACCCCAGACCGACCTCTTGAGTCTCAACTCCAAGTCAGCCGCAACTTTAATTATCCGTGGATTGGGAGCTAGTTTGGACAACG | |
| | CATCAGTATAATATAACTTTACGGTTCCATTATCAGACGCTATTGCAAGAACTTCCTTTCCATTGATCTCGCCAATGCGGCAGTAATTGAT | |
| | ATCGTAGGGTAGGTCTGGAAAGACGCTGGCGCTTGTGTCCCATTCTGCAGGAATCTCTGGCACGGTGCTAATGGTAGTTATCCAACGG | |
| | AGCTGAGGTAGTCGATATATCTGGATATGCCGCCTATAGGATAAAAACAGGAGAGGGTGAACCTTGCTTATGGCTACTAGATTGTTCTT | |
| | GTACTCTGAATTCTCATTATGGGAAACTAAACTAATCTCATCTGTGTGTTGCAGTACTATTGAATCGTTGTAGTATCTACCTGGAGGGCA | |
| | TTCCATGAATTAGTGAGATAACAGAGTTGGGTAACTAGAGAGAATAATAGACGTATGCATGATTACTACACAACGGATGTCGCACTCTT | |
| | TCCTTAGTTAAAACTATCATCCAATCACAAGATGCGGGCTGGAAAGACTTGCTCCCGAAGGATAATCTTCTGCTTCTATCTCCCTTCCTCA | |
| | TATGGTTTCGCAGGGCTCATGCCCCTTCTTCCTTCGAACTGCCCGATGAGGAAGTCCTTAGCCTATCAAAGAATTCGGGACCATCATCGA | |
| | TTTTTAGAGCCTTACCTGATCGCAATCAGGATTTCACTACTCATATAAATACATCGCTCAAAGCTCCAACTTTGCTTGTTCATACAATTCTT | |
| | GATATTCACAGG | |
| 8 | CTTCAGTAATGTCTTGTTTCTTTTGTTGCAGTGGTGAGCCATTTTGACTTCGTGAAAGTTTCTTTAGAATAGTTGTTTCCAGAGGCCAAAC | pILV5/ |
| | ATTCCACCCGTAGTAAAGTGCAAGCGTAGGAAGACCAAGACTGGCATAAATCAGGTATAAGTGTCGAGCACTGGCAGGTGATCTTCTG | pEM72 |
| | AAAGTTTCTACTAGCAGATAAGATCCAGTAGTCATGCATATGGCAACAATGTACCGTGTGGATCTAAGAACGCGTCCTACTAACCTTCG | |
| | CATTCGTTGGTCCAGTTTGTTGTTATCGATCAACGTGACAAGGTTGTCGATTCCGCGTAAGCATGCATACCCAAGGACGCCTGTTGCAA | |
| | TTCCAAGTGAGCCAGTTCCAACAATCTTTGTAATATTAGAGCACTTCATTGTGTTGCGCTTGAAAGTAAAATGCGAACAAATTAAGAGA | |
| | TAATCTCGAAACCGCGACTTCAAACGCCAATATGATGTGCGGCACACAATAAGCGTTCATATCCGCTGGGTGACTTTCTCGCTTTAAAA | |
| | AATTATCCGAAAAAATTTTCTAGAGTGTTGTTACTTTATACTTCCGGCTCGTATAATACGACAAGGTGTAAGGAGGACTAAACC | |

TABLE 2
Terminator sequences

| SEQ | | |
|---|---|---|
| ID | | ANNO- |
| NO. | SEQUENCE | TATION |
| 9 | TCAAGAGGATGTCAGAATGCCATTTGCCTGAGAGATGCAGGCTTCATTTTTGATACTTTTTTATTTGTAACCTA | tAOX1 |
| | TATAGTATAGGATTTTTTTTGTCATTTTGTTTCTTCTCGTACGAGCTTGCTCCTGATCAGCCTATCTCGCAGCAG | |
| | ATGAATATCTTGTGGTAGGGGTTTGGGAAAATCATTCGAGTTTGATGTTTTTCTTGGTATTTCCCACTCCTCTT | |
| | CAGAGTACAGAAGATTAAGTGAAACCTTCGTTTGTGCG | |
| 10 | AATTGACACCTTACGATTATTTAGAGAGTATTTATTAGTTTTATTGTATGTATACGGATGTTTTATTATCTATTTATGCCCTTATATTCTGT | tAOD1 |
| | AACTATCCAAAAGTCCTATCTTATCAAGCCAGCAATCTATGTCCGCGAACGTCAACTAAAAATAAGCTTTTATGCTCTTCTCTCTTTTTTT | |
| | CCCTTCGGTATAATTATACCTTGCATCCACAGATTCTCCTGCCAAATTTTGCATAATCCTTTACAACATGGCTATATGGGAGCACTTAGCG | |
| | CCCTCCAAAACCCATATTGCCTACGCATGTATAGGTGTTTTTTCCACAATATTTTCTCTGTGCTCTCTTTTTATTAAAGAGAAGCTCTATAT | |
| | CGGAGAAGCTTCTGTGGCCGTTATATTCGGCCTTATCGTGGGACCACATTGCCTGAATTGGTTTGCCCCGGAAGATTGGGGAAACTTG | |
| | GATCTGATTACCTTAGCTGCA | |

TABLE 3
Signal peptide sequences

| SEQ | | |
|---|---|---|
| ID | | |
| NO. | SEQUENCE | ANNOTATION |
| 11 | ATGAGATTCCCATCTATTTTCACCGCTGTCTTGTTCGCTGCCTCCTCTGCATTGGCTGCCCCTGTTAACACTACCACTGAAGACGA | alpha mating |
| | GACTGCTCAAATTCCAGCTGAAGCAGTTATCGGTTACTCTGACCTTGAGGGTGATTTCGACGTCGCTGTTTTGCCTTTCTCTAAC | factor |
| | TCCACTAACAACGGTTTGTTGTTCATTAACACCACTATCGCTTCCATTGCTGCTAAGGAAGAGGGTGTCTCTCTCGAGAAAAGA | secretion |
| | GAGGCCGAAGCT | signal (seq 1) |
| 12 | ATGAGATTCCCTTCTATTTTCACTGCTGTTTTGTTCGCTGCTTCTTCTGCTTTGGCTGCTCCAGTTAACACTACTACCGAAGACGA | alpha mating |
| | AACTGCTCAAATTCCTGCTGAAGCTGTTATTGGTTACTCTGACTTGGAAGGTGACTTCGACGTTGCTGTTTTGCCATTCTCTAACT | factor |
| | CTACTAACAACGGTTTGTTGTTCATTAACACTACTATTGCTTCTATTGCTGCTAAGGAAGAAGGTGTTTCTTTGGACAAGAGAGA | secretion |
| | AGCTGAAGCT | |

TABLE 4
Protein fused to secretion signal

| SEQ | | |
|---|---|---|
| ID | | |
| NO. | SEQUENCE | ANNOTATION |
| 13 | GCTGAAGTAGACTGCTCAAGATTTCCAAATGCTACTGACAAGGAAGGAAAGGATGTCCTCGTATGTAACAAGGACCTTAGACCC | OVD (Seq 1) |
| | ATTTGCGGTACGGATGGCGTGACATACACTAATGATTGTTTACTATGTGCCTATAGCATTGAGTTCGGTACAAACATCTCCAAAG | |
| | AGCACGATGGAGAATGTAAAGAGACTGTCCCTATGAACTGTTCCTCTTACGCAAATACAACTTCAGAGGACGGTAAGGTGATG | |
| | GTCTTGTGTAACAGGGCTTTCAATCCAGTTTGTGGTACTGACGGTGTTACTTACGATAACGAATGTCTGTTGTGTGCTCATAAAG | |
| | TTGAGCAAGGAGCATCTGTTGATAAAAGACACGATGGTGGATGCCGTAAGGAATTGGCCGCAGTTTCGGTGGACTGCTCCGAA | |
| | TATCCAAAACCTGACTGTACCGCTGAGGATCGTCCTCTGTGCGGAAGTGACAACAAGACCTATGGTAATAAGTGTAATTTCTGTA | |
| | ATGCTGTTGTTGAAAGCAATGGTACATTAACATTGTCTCATTTTGGTAAatgttaa | |
| 14 | GCAGAAGTTGACTGTTCTCGTTTCCCAAATGCTACTGACAAGGAAGGAAAAGACGTCTTGGTGTGTAACAAGGATTTGAGGCCA | OVD (seq 2) |
| | ATTTGTGGTACAGATGGTGTGACTTACACTAATGATTGTCTACTTTGCGCATATAGCATCGAGTTTGGAACCAATATCTCAAAAG | |
| | AGCACGACGGTGAATGTAAAGAGACTGTCCCAATGAACTGTTCTTCCTACGCTAATACAACCTCCGAGGATGGTAAAGTAATGG | |
| | TTTTGTGCAACAGAGCCTTTAATCCTGTTTGTGGCACGGATGGAGTCACTTATGATAATGAATGTCTCCTGTGCGCCCACAAGGT | |
| | AGAACAAGGTGCTAGCGTTGATAAGCGTCATGACGGTGGATGTAGAAAGGAATTAGCTGCTGTGTCTGTTGATTGTTCAGAAT | |
| | ATCCCAAGCCTGACTGTACAGCTGAGGACAGACCTCTGTGCGGTTCCGACAACAAAACATACGGAAACAAATGCAACTTCTGTA | |
| | ATGCAGTGGTTGAGTCGAATGGAACATTGACTTTAAGTCATTTCGGTAAATGT | |
| 15 | CTAGTAAAGGTGCCTCTAGTTAGAAAGAAGAGTCTGAGACAAAACCTAATTAAGAACGGAAAACTGAAGGATTTCTTAAAAAC | PGA |
| | GCATAAACATAACCCCGCCTCCAAATACTTTCCTGAAGCAGCCGCTTTAATAGGCGACGAACCTTTAGAAAATTACTTAGATACC | |
| | GAGTATTTCGGCACTATTGGTATTGGTACGCCCGCACAAGATTTCACGGTAATCTTCGACACCGGCAGTTCAAATTTATGGGTGC | |
| | CCTCCGTGTATTGTAGTAGTTTGGCTTGCTCCGACCATAATCAGTTCAACCCCGATGATTCCTCCACGTTCGAGGCCACGAGTCA | |
| | AGAATTGAGTATAACCTACGGCACCGGTTCCATGACAGGCATCCTAGGATACGATACAGTACAAGTCGGCGGCATTTCCGACAC | |
| | CAATCAGATATTTGGCCTAAGTGAGACCGAGCCCGGATCTTTCTTGTACTACGCCCCTTTCGACGGAATCTTGGGTCTAGCTTAT | |
| | CCTAGTATATCTGCATCCGGAGCTACACCCGTGTTTGACAACCTATGGGATCAGGGCCTTGTCTCCCAGGATCTATTCTCAGTCT | |
| | ACCTGAGTAGTAATGATGATTCAGGCTCAGTAGTGTTGCTAGGCGGAATTGATTCTAGTTACTACACAGGTTCTCTGAACTGGG | |
| | TTCCTGTCAGTGTAGAGGGCTATTGGCAGATCACACTGGATTCCATAACTATGGATGGAGAGACCATCGCCTGCTCCGGCGGTT | |
| | GTCAGGCAATAGTGGATACCGGAACCAGTCTGTTGACTGGCCCTACCTCTGCCATAGCTAATATACAAAGTGATATAGGAGCAT | |
| | CTGAGAACTCTGACGGCGAGATGGTAATCTCTTGTTCTAGTATCGATTCATTACCTGACATAGTTTTTACCATAAATGGTGTTCAA | |
| | TACCCCCTAAGTCCTTCCGCCTATATCTTGCAAGATGATGACTCATGTACAAGTGGCTTTGAAGGTATGGATGTACCCACGTCAT | |
| | CAGGTGAGCTTTGGATACTGGGCGATGTGTTTATCAGGCAATACTACACCGTGTTCGATAGGGCTAACAACAAGGTGGGTCTA | |
| | GCACCTGTTGCATAA | |
| 16 | MRFPSIFTAVLFAASSALAAPVNTTTEDETAQIPAEAVIGYSDLEGDFDVAVLPFSNSTNNGLLFINTTIASI | OVA |
| | AAKEEGVSLDKREAEAGSIGAASMEFCFDVFKELKVHHANENIFYCPIAIMSALAMVYLGAKDSTRTQINK | |
| | VVRFDKLPGFGDSIEAQCGTSVNVHSSLRDILNQITKPNDVYSFSLASRLYAEERYPILPEYLQCVKELYRGG | |
| | LEPINFQTAADQARELINSWVESQTNGIIRNVLQPSSVDSQTAMVLVNAIVFKGLWEKAFKDEDTQAMP | |
| | FRVTEQESKPVQMMYQIGLFRVASMASEKMKILELPFASGTMSMLVLLPDEVSGLEQLESIINFEKLTEW | |
| | TSSNVMEERKIKVYLPRMKMEEKYNLTSVLMAMGITDVFSSSANLSGISSAESLKISQAVHAAHAEINEAG | |
| | REVVGSAEAGVDAASVSEEFRADHPFLFCIKHIATNAVLFFGRCVSP | |

TABLE 5
Selection and other markers

| SEQ | | |
|---|---|---|
| ID | | |
| NO. | SEQUENCE | ANNOTATION |
| 17 | AGTGAAAACGAAAAGTGAAAATATCCTGAGGACGACTTTTATTCTTTTGGCTGGTGCTAGCGCTGCATGTTCGTTACTAGCCGT | Ura3 cassette |
| | TCAATACCCATTTTCTAAAGTTCAGTCAATACATTTAGCAAGGTTGGAAGCGTTGGATATTTTCAACGAGAACTCCTGGGATAAG | (promoter, |
| | AAAAGTAAATTCCGTCTATATTACCGATCATACTTAGATACATTCCACCAGTTGGTGAGCTTACATGAGAAGTCTAAACTATCTT | gene, |
| | GGACCCGCTGGTTTTATAAAGGGTTCGTTAGGAATGCGTTAACTACCATTCCAGCAACATCCGTGGGGCTTCTGGTGTTTGAAA | terminator) |
| | TACTGCGTCAAAAATTGAGCGATGAAATTGAAGATCGATTCAGTTGAATCGCCCGAAACAATTGATCCCCTGTACATACTTGTA | |
| | ATTTACCTCAGAATATTTTGGTAAGCTTCCCACCCAGCTTTTCTATACCGTTCACCTTCTTTTAAGGGATCTCTGCCCTTGCCAAA | |
| | CAAACCACGACCTACGATTATGATGTCAGTGCCAGTGGAAAATACTTGACTCACTGTTCGATATTGTTGGCCTAGAGCATCACCA | |
| | GTGTCATCCAAACCAACACCTGGTGTCATAATAATCCAATCGAACCCTTCATCTTGTCCTCCCATAGAATTTTGAGCAATAAACCC | |
| | AATGACGAATTCCTTGTCTGATTTTGCAATTTCTACAGTTTCTTCGGTGTACTTACCATGGGCAATTGATCCCTTTGACGACAGTT | |
| | CAGCCAACATCAATAGTCCCCTTGGTTGATCTGTTGTCTCAGTGGCTGCCTCCTTTAGACCCTTTACAATTCCACTACCAATGACA | |
| | CCATGAGCATTTGTAATATCTGCCCATTGTGCAATCTTGTAGACACCTCCTTGATATTGATGCTTGACAGTGTTGCCTATATCAGC | |
| | AAACTTTCTGTCCTCAAAAATTAAAAACTTGTGTTTCTTTGATAGTTCCAATAAAGGCAGAATAGTTCCATCATACGTGAAGTCA | |
| | TCAATTATGTCGATATGAGTCTTGGCCAAACAGATAAATGGGCCCAATTTATCTAGAAGCTCCAATAATTCTTTAGTTGTTCTCA | |
| | CGTCGACTGATGCGCATAGGTTACTCTGTTTCTGTTCCATAAGCGCAAACAGTCGTCGTGCCACAGGTGATTGATGAGTATTTG | |
| | CTCTCTCGGCATAACTGCGAGCCATTGTCTAGGTATCTATCCCTTTGATCAGGTTGATGTTAACTCATTAGAGTGGATCAATGCG | |
| | AAGGATAGGTGCGACGTGTACCGTCCAAAAAACTTTTTTCTTCAATCTTGACAAAAACTGGTAACAGAGAGAGCAAGTGCTAAC | |
| | TCTACCCCAACCAAGTACATCACAAAATGGACGCATTGAACGCTAAAGAACAACAAGAGTTCCAGAAACTCGTTGAACAAAAA | |
| | CAAATGAAAGACTTCATGCGTCTTTACTCCGATTTGGTTAGCAAATGTTTTACAGACTGTGTCAATGATTTTACATCTAACAAGTT | |
| | GACTTCTAAGGAGGAAGGCTGCATCAACAAGTGTGCAGAAAAGTTCCTCAAGCACAGTGAGAGAGTTGGTCAACGTTTCCAAG | |
| | AACAAAACCAACTTATGATGCAACAGCTAAGACGTTAACCCCATATTTTTGTACATAAAGTTCATTGTCCAGGACTAATCCAGAC | |
| | TTTCTCTGAACAGCTCTATAATCTTAGTAGTTTCTTCCATCATTTCAATCGTTAGCTTCGAAACATCACTGTCTTCATCGTTATAG | |
| | ATGATGGCATTTGTAAACATGATCTGTAGAACTTTAGTCAATTCGTCAAAGGTGGTTATCTCCCCATTTCTGCAGTGCTTGAGAAT | |
| | GGTCTTCAGATCTTGA | |
| 18 | ATGGCTAAACTCACCTCTGCTGTTCCAGTCCTGACTGCTCGTGATGTTGCTGGTGCTGTTGAGTTCTGGACTGATAGACTCGGTT | Zeocin |
| | TCTCCCGTGACTTCGTAGAGGACGACTTTGCCGGTGTTGTACGTGACGACGTTACCCTGTTCATCTCCGCAGTTCAGGACCAGG | resistance |
| | TTGTGCCAGACAACACTCTGGCATGGGTATGGGTTCGTGGTCTGGACGAACTGTACGCTGAGTGGTCTGAGGTCGTGTCTACC | |
| | AACTTCCGTGATGCATCTGGTCCAGCTATGACCGAGATCGGTGAACAGCCCTGGGGTCGTGAGTTTGCACTGCGTGATCCAGCT | |
| | GGTAACTGCGTGCATTTCGTCGCAGAAGAGCAGGACTAA | |
| 19 | ATGGGTAAAGAGAAAACGCACGTCAGTCGTCCAAGATTGAACTCCAATATGGATGCAGACCTGTACGGTTACAAATGGGCTAG | G418/Kanamycin |
| | AGATAACGTTGGACAATCTGGTGCAACTATATATAGATTGTATGGGAAGCCAGACGCACCAGAGTTGTTTCTAAAGCATGGGA | resistance |
| | AAGGCTCTGTTGCTAATGATGTGACTGATGAAATGGTACGTTTGAATTGGCTAACAGAGTTTATGCCCTTGCCTACTATTAAGC | |
| | ATTTTATTCGTACTCCCGATGACGCTTGGTTGCTAACCACCGCAATTCCTGGTAAAACTGCCTTTCAAGTTCTGGAAGAATACCC | |
| | AGATTCCGGTGAAAACATCGTTGACGCCTTGGCTGTTTTCCTGCGAAGACTTCACTCTATTCCCGTATGTAATTGTCCCTTTAATT | |
| | CAGACAGAGTTTTTAGATTGGCTCAGGCTCAATCTAGGATGAATAATGGTTTGGTTGATGCAAGTGACTTCGATGACGAAAGA | |
| | AACGGTTGGCCTGTCGAGCAGGTGTGGAAGGAAATGCATAAGTTACTTCCATTTTCTCCTGATTCTGTTGTAACCCACGGTGAT | |
| | TTTTCCCTAGACAACCTTATATTCGATGAGGGCAAGTTGATTGGTTGTATTGACGTCGGCAGAGTGGGTATCGCCGATAGGTAT | |
| | CAAGATTTAGCAATACTGTGGAATTGTCTAGGAGAATTTTCACCCAGTCTGCAAAAGAGATTGTTCCAGAAATACGGAATTGAC | |
| | AACCCCGATATGAATAAGTTGCAGTTTCATTTGATGTTGGACGAGTTCTTCTAA | |
| 20 | ATGGGAAAGAAACCAGAGCTGACCGCAACGAGTGTCGAAAAATTTCTTATTGAAAAATTTGATAGTGTGTCCGATTTAATGCA | Hygromycin |
| | GCTTAGTGAAGGCGAAGAGTCACGTGCTTTCTCATTCGACGTTGGTGGACGTGGCTACGTTTTGAGAGTTAATAGTTGTGCAG | resistance |
| | ATGGCTTTTATAAGGATCGTTATGTATACCGTCATTTTGCTAGTGCAGCCCTGCCAATCCCAGAGGTTTTAGATATAGGTGAGTT | |
| | TAGTGAGTCTCTTACTTATTGTATTAGTCGTAGAGCCCAAGGTGTTACCCTTCAGGATTTGCCAGAGACTGAGCTTCCTGCTGTA | |
| | TTGCAACCTGTCGCTGAGGCTATGGACGCCATTGCCGCAGCAGATTTATCTCAAACGTCAGGTTTCGGCCCCTTCGGCCCACAA | |
| | GGCATCGGACAGTACACAACGTGGCGTGACTTTATCTGTGCCATCGCTGACCCTCATGTCTACCACTGGCAAACGGTCATGGAT | |
| | GACACGGTGTCCGCCTCTGTGGCCCAAGCATTGGATGAACTGATGCTTTGGGCTGAGGATTGTCCCGAAGTCCGTCACCTGGTT | |
| | CACGCTGACTTCGGCTCCAACAATGTTTTGACCGACAATGGCCGTATCACCGCTGTCATCGACTGGTCTGAGGCAATGTTTGGC | |
| | GACTCTCAGTATGAAGTCGCCAATATATTTTTTTGGAGACCCTGGTTGGCATGCATGGAACAGCAAACTCGTTACTTTGAAAGA | |
| | CGTCATCCAGAGTTAGCTGGTAGTCCACGTCTGCGTGCTTACATGTTGCGTATCGGCTTAGACCAACTGTATCAGTCACTTGTCG | |
| | ATGGTAACTTTGATGACGCAGCATGGGCACAAGGACGTTGTGACGCTATTGTACGTTCAGGTGCAGGCACGGTCGGCCGTACA | |
| | CAAATTGCACGTAGAAGTGCAGCAGTCTGGACCGATGGTTGTGTTGAGGTCCTTGCAGATTCAGGAAATAGACGTCCATCTACT | |
| | CGTCCTCGTGCTAAGGAATAA | |
| 21 | GGAATTGTGAGCGGATAACAATTCC | LacOperator |
| 22 | ATGGCCAATTTACTGACCGTACACCAAAATTTGCCTGCATTACCGGTCGATGCAACGAGTGATGAGGTTCGCAAGAACCTGATG | Cre |
| | GACATGTTCAGGGATCGCCAGGCGTTTTCTGAGCATACCTGGAAAATGCTTCTGTCCGTTTGCCGGTCGTGGGCGGCATGGTG | recombinase |
| | CAAGTTGAATAACCGGAAATGGTTTCCCGCAGAACCTGAAGATGTTCGCGATTATCTTCTATATCTTCAGGCGCGCGGTCTGGC | |
| | AGTAAAAACTATCCAGCAACATTTGGGCCAGCTAAACATGCTTCATCGTCGGTCCGGGCTGCCACGACCAAGTGACAGCAATG | |
| | CTGTTTCACTGGTTATGCGGCGCATCCGAAAAGAAAACGTTGATGCCGGTGAACGTGCAAAACAGGCTCTAGCGTTCGAACGC | |
| | ACTGATTTCGACCAGGTTCGTTCACTCATGGAAAATAGCGATCGCTGCCAGGATATACGTAATCTGGCATTTCTGGGGATTGCT | |
| | TATAACACCCTGTTACGTATAGCCGAAATTGCCAGGATCAGGGTTAAAGATATCTCACGTACTGACGGTGGGAGAATGTTAATC | |
| | CATATTGGCAGAACGAAAACGCTGGTTAGCACCGCAGGTGTAGAGAAGGCACTTAGCCTGGGGGTAACTAAACTGGTCGAGC | |
| | GATGGATTTCCGTCTCTGGTGTAGCTGATGATCCGAATAACTACCTGTTTTGCCGGGTCAGAAAAAATGGTGTTGCCGCGCCAT | |
| | CTGCCACCAGCCAGCTATCAACTCGCGCCCTGGAAGGGATTTTTGAAGCAACTCATCGATTGATTTACGGCGCTAAGGATGACT | |
| | CTGGTCAGAGATACCTGGCCTGGTCTGGACACAGTGCCCGTGTCGGAGCCGCGCGAGATATGGCCCGCGCTGGAGTTTCAATA | |
| | CCGGAGATCATGCAAGCTGGTGGCTGGACCAATGTAAATATTGTCATGAACTATATCCGTAACCTGGATAGTGAAACAGGGGC | |
| | AATGGTGCGCCTGCTGGAAGATGGCGATTAA | |
| 23 | ATAACTTCGTATAATGTATGCTATACGAACGGTA | lox71 (reverse |
| | | complement) |
| 24 | TACCGTTCGTATAATGTATGCTATACGAAGTTAT | lox66 (reverse |
| | | complement) |
| 25 | CCCGTAGAAAAGATCAAAGGATCTTCTTGAGATCCTTTTTTTCTGCGCGTAATCTGCTGCTTGCAAACAAAAAAACCACCGCTAC | pUC origin of |
| | CAGCGGTGGTTTGTTTGCCGGATCAAGAGCTACCAACTCTTTTTCCGAAGGTAACTGGCTTCAGCAGAGCGCAGATACCAAATA | replication |
| | CTGTTCTTCTAGTGTAGCCGTAGTTAGGCCACCACTTCAAGAACTCTGTAGCACCGCCTACATACCTCGCTCTGCTAATCCTGTTA | |
| | CCAGTGGCTGCTGCCAGTGGCGATAAGTCGTGTCTTACCGGGTTGGACTCAAGACGATAGTTACCGGATAAGGCGCAGCGGTC | |
| | GGGCTGAACGGGGGGTTCGTGCACACAGCCCAGCTTGGAGCGAACGACCTACACCGAACTGAGATACCTACAGCGTGAGCTA | |
| | TGAGAAAGCGCCACGCTTCCCGAAGGGAGAAAGGCGGACAGGTATCCGGTAAGCGGCAGGGTCGGAACAGGAGAGCGCAC | |
| | GAGGGAGCTTCCAGGGGGAAACGCCTGGTATCTTTATAGTCCTGTCGGGTTTCGCCACCTCTGACTTGAGCGTCGATTTTTGTG | |
| | ATGCTCGTCAGGGGGGCGGAGCCTATGGAAAAACGCCAGCAACGCGGCCTTTTTACGGTTCCTGGCCTTTTGCTGGCCTTTTGC | |
| | TCACA | |

TABLE 6
Helper protein sequences

| SEQ | | |
|---|---|---|
| ID | | ANNO- |
| NO. | SEQUENCE | TATION |
| 26 | MDREQGILPQDPFSNSVHVPKLRASSGGQPQKPVIQNSAPATARMLRNASSSTSAALLKELNTHEHSQRQHTPQKQPSLDAPAALV | Kin2 |
| | PVESATKQFHRTSIGDWEFSNTIGAGSMGKVKVAKHRVTHEVCAIKIVIRSAKIWQRNHQNDPEPETEEKRKKLRDEYKKELERDERT | |
| | VREAALGKIMYHPNICRLFECYTMSNHYYMLFEIVQGVQLLDYIVSHGKLKETRVRQFARSIASALDYCHSNNIVHRDLKIENIMINNK | |
| | GEIKLIDFGLSNMYDRRNLLKTFCGSLYFAAPELLSCRPYIGPEIDVWSFGVVLFVLVSGKVPFDDDSVPKLHAKIKRGKVEYPEFISPLC | |
| | HSLLSQMLVVNPDHRVTLKAAMEHPWMTLGFAGPPSNYLPQRSPIVLPLDLSVVREIANLGLGNEEQIARDITNLISSREYEACVER | |
| | WKLDQQKANIKGYSARDDSAIIAFHPLLSTYYLVDEMRKRKLAKGALKGQTSVLDTVKVSPDIPKTPAIPQKLETTDVEQPLLATVPPA | |
| | YTSPHGQPAELEAMIEPAQPLSSAHPFEMDMTQQQHASRKTHIKHAPERQDRGGYNVHKNNSGGLNSLFRRLSGKRPHKNEAEW | |
| | EPSSPPPQVHPFSVNDADRTSVRGVSPITQPAAVKNVTSNNSKNYLDPVDDSKLVRRVGSLRITNKEKQQVTSDFPRLPNFTIPEQPP | |
| | KNAPIPIHAQPTTTGTTFQSNDHEIKKKLQASTSPNEQRGPPTLAPSQQRRLHPTARAKSLGHSRKQSLNFKFGGPANNQLPALPTKE | |
| | NYDVFEDAQITDNNLLNPEGKYSANTNVHIKPMTESQILFEAEHAPPGTMPSVEYPRTLFLKGFFSVQTTSSKPLPVIRYNIIAALCKLN | |
| | IQFTEVNGGFVCVYRKTENLQIGDIRSPVIESRVTDDTDSDVANSSKLSSSSTANTRVNVIEDDSSSPSSARLKHRRKFSLGNGILNHIRK | |
| | PTLDGTEFDDYDATVNTPVTPAPANVHSRSSSYHTESDNESMESLHDIRGGSDMILKNVPERNARQIDTVKEEETDDDDLGSINEGS | |
| | THRTPLKFEIHIVKVPLVGLYGVRFKKILGNAWIYKRLASKLLQELNL* | |
| 27 | MGKLAQLVLHPLELRAAIQFKFFKQSLHPRQPTNERETLKHCYELLALTSRSFCTVILELNPELRNAIMIFYLVLRALDTVEDDMTIKPDI | ERG9 |
| | KIPLLRSFDEKLNLKSWSFDGNSPDEKDRQVLVDFTDVLEEYHRLKPVYQDVIKDITHKMGNGMADYITDEEFNLNGVATVKDYDLY | |
| | CHYVAGLVGEGLTHLIVEAGFGDPKLEDNMQLSESMGLFLQKTNIIRDYREDLDDGRSFWPKEIWSKYADSLSDFSKRENYEKGLDCI | |
| | SELVLNTMDHIKDVLVYLSSVYDFSSYNFCVIPQVMAIATLATVFRNEKVFETNVKIRKGTTCYLILKARTFEGACEIFSYYLRQIHHSCP | |
| | ITDANYIKIGIKCGELEQFLESLNPAPHVPPGATIPQTPHFVKAERKRKLDRELVPTLAIESLKCDVFLSLVALGFLGVIYSISS* | |
| 28 | MQFNWDIKTVASILSALTLAQASDQEAIAPEDSHVVKLTEATFESFITSNPHVLAEFFAPWCGHCKKLGPELVSAAEILKDNEQVKIAQ | PDI1 |
| | IDCTEEKELCQGYEIKGYPTLKVFHGEVEVPSDYQGQRQSQSIVSYMLKQSLPPVSEINATKDLDDTIAEAKEPVIVQVLPEDASNLESN | |
| | TTFYGVAGTLREKFTFVSTKSTDYAKKYTSDSTPAYLLVRPGEEPSVYSGEELDETHLVHWIDIESKPLFGDIDGSTFKSYAEANIPLAYY | |
| | FYENEEQRAAAADIIKPFAKEQRGKINFVGLDAVKFGKHAKNLNMDEEKLPLFVIHDLVSNKKFGVPQDQELTNKDVTELIEKFIAGEA | |
| | EPIVKSEPIPEIQEEKVFKLVGKAHDEVVFDESKDVLVKYYAPWCGHCKRMAPAYEELATLYANDEDASSKVVIAKLDHTLNDVDNVD | |
| | IQGYPTLILYPAGDKSNPQLYDGSRDLESLAEFVKERGTHKVDALALRPVEEEKEAEEEAESEADAHDEL* | |
| 29 | MPAVGIDLGTTYSCVAHFANDRVEIIANDQGNRTTPSFVAFTDTERLIGDAAKNQAAMNPANTVFDAKRLIGRKFSDAETQADIKHF | SSA1 |
| | PFKVVDKGGKPNIQVEFKGETKVFTPEEISSMVLTKMKDTAEQFLGDKVNDAVVTVPAYFNDSQRQATKDAGLIAGLNVMRIINEPT | |
| | AAAIAYGLDKKAEGEKNVLIFDLGGGTFDVSLLSIEDGIFEVKATAGDTHLGGEDFDNRLVNHFIAEFKRKNKKDLSSNQRALRRLRTA | |
| | CERAKRTLSSSAQTSIEIDSLFEGVDFYTSLTRARFEELCGDLFRSTIEPVEKVLKDAKLDKSQVNEIVLVGGSTRIPKVQKLVSDFFNGK | |
| | EPNRSINPDEAVAYGAAVQAAILSGDTSSKTQDLLLLDVAPLSLGIETAGGIMTKLIPRNSTIPTKKSETFSTYADNQPGVLIQVYEGERA | |
| | KTADNNLLGKFELSGIPPAPRGVPQIEVTFDMDANGILNVSAVEKGTGKAQQITITNDKGRLSKEDIEAMISEAEKYKDEDEKEAARIQ | |
| | ARNALESYSFSLKNTLNEKEVGEKLDAADKESLTKAIDETTSWIDENQTATTEEFEAKQKELEGVANPIMTKFYQANGGAPGGAAPG | |
| | GFPGAAGAGAEAPGADGPTVEEVD* | |
| 30 | MGKSIGIDLGTTYSCVAHFANDRVEIIANDQGNRTTPSFVAFTDTERLIGDAAKNQAAMNPANTVFDAKRLIGRKFDDPETQADIKH | SSA4 |
| | FPFKVINKGGKPNIQVEFKGETKVFSPEEISSMVLTKMKDTAEQYLGEKINDAVVTVPAYFNDSQRQATKDAGLIAGLNVQRIINEPT | |
| | AAAIAYGLDKKDAGHGEHNILIFDLGGGTFDVSLLSIDEGIFEVKATAGDTHLGGEDFDNRLVNHFIAEFKRKTKKDLSTNQRSLRRLR | |
| | TACERAKRTLSSSAQTSIEIDSLFEGIDFYTSITRARFEELCADLFRSTIEPVERVLKDSKLDKSQVHEIVLVGGSTRIPKVQKLVSDFFN | |
| | GKEPNKSINPDEAVAYGAAVQAAILSGDTSSKTQDLLLLDVAPLSLGIETAGGIMTKLIPRNSTIPAKKSEIFSTYADNQPGVLIQVFEGE | |
| | RTRTKDNNLLGKFELSGIPPAPRGVPQIEVTFDMDANGILNVSAVEKGTGKTQKITITNDKGRLSKEDIERMVSEAEKFKDEDEKEAERV | |
| | AAKNGLESYAYSLKNSAAESGFKDKVGEDDLAKLNKSVEETISWLDESQSASTDEYKDRQKELEEVANPIMSKFYGAAGGAPGGAPG | |
| | GFPGGFPGGAGAAGGAPGGAAPGGDSGPTVEEVD* | |
| 31 | MADGVFQGAIGIDLGTTYSCVATYDSAVEIIANEQGNRVTPSFVAFTPEERLIGDAAKNQAALNPKNTVFDAKRLIGRAFDDESVQK | SSB1 |
| | DIKSWPFKVVNDNGNPLIEVEYLGETKQFSPQEISSMVLTKMKEVAEAKIGQKVEKAVVTVPAYFNDAQRQATKDAGAISGLNVLRII | |
| | NEPTAAAIAYGLGAGKSEEEKHVLIFDLGGGTFDVSLLHIAGGVFTVKATAGDTHLGGQDFDTNLLEFFKKEFQKKTGKDISDDARAL | |
| | RRLRTACERAKRTLSSVAQTTVEVDSLFDGEDFTAEISRAKFEAINADLFKSTLEPVEQVLKDSKIEKSKVDDVVLVGGSTRIPKVQKLLS | |
| | DFFDGKQLEKSINPDEAVAYGAAVQGAILTGQSTSEETKDLLLLDVIPLSLGVAMQGNVFAPVVPRNTTVPTIKRRTFTTVDDHQTTV | |
| | QFPVYQGERVNCSENTLLGEFDLKNIPPMSAGEPVLEAIFEIDANGILKVTAVEKSTGRSANITISNSIGRLSSSEIEKMINDADKFKKAD | |
| | EDFANRHESKQKLEAYVSSIESTITDPILSSKLKRSAKDKIESALSDALAALELEDASGDDFRKAELALKRVVTKAMATR* | |
| 32 | MRDGEFFSFSLNSVARPMQSFFGKTNILANLRRNSETMSVPFGVDLGNNNTVIGVARNRGIDILVNEVSNRQTPSIVGFGAKSRAIG | SSE1 |
| | ESGKTQQNSNLKNTVEHLVRILGLPADSPDYEIEKKFFTSPLIEKDNEILSEVNFQGKKTTFTPIQLVAMYLNKIKNTAIKETKGKFTDIC | |
| | LAVPVWFTEKQRSAASDACKVAGLNPVRIVNDITAAAVGYGVFKTDLPEDEPKKVAIVDIGHSTYSVLIAAFKKGELKVLGSASDKHFG | |
| | GRDFDYAITKHFAEEFKSKYKIDITQNPKAWSRVYTAAERLKKVLSANTTAPFNVESVMNDVDVSSSLTREELEKLVQPLLDRAHIPVE | |
| | RALAMAGLKAEDVDTVEVVGGCTRVPTLKATLSEVFGKPLSFTLNQDEAIARGAAFICAMHSPTLRVRPFKFEDVNPYSVSYYWDKD | |
| | PAAEDDDHLEVFPVGGSFPSTKVITLYRSQDFNIEARYTDKNALPAGTQEFIGRWSIKGVVVNEGEDTIQTKIKLRNDPSGFHIVESAY | |
| | TVEKKTIQEPIEDPEADEDAEPQYRTVEKLVKKNDLEITGQTLHLPDELLNSYLETEAALEVQDKLVADTEERKNALEEYIYELRGKLEDQ | |
| | YKEFASEQEKTKLTAKLEKAEEWLYDEGYDSTKAKYIAKYEELASIGNVIRGRYLAKEEEKKQAIREKEESKKASAIAEKMAAERASREA | |
| | AGSTNEQAQKNEENTKDADGDVSMNQDELD* | |
| 33 | MPVDSSHKTASPLPPRKRAKTEEEKEQRRVERILRNRRAAHASREKKRRHVEFLENHVVDLESALQESAKATNKLKQIQDIIVSRLEAL | HAC1 |
| | GGTVSDLDLAVPEVDFPKFSDLELSTDLSSSTKSEKASTSTCRSSTEDLDEDGVAEYDDEEDEELPRKKNVLNDKSKNRTIKQEKLNELP | |
| | SPLSSDFSDVDEEKSTLTHFQLQQQQQQQPVDNYVSTPLSLPEDSIDFINPGSLKIESDENFLLGSSTLQIKHENDTEYIPTAPSGSINDF | |
| | FNSYDISESNRLHHPAAPFTANAFDLNDFVFFQE* | |
| 34 | MNGKHLLLQVLLVQLVAAVLDTQVGYIDWLVTSTGSFLDLSSCLFNYEQIYCLTEANDLIGLDSDAQITYRLHLDGPDQGKLTKLNNK | EMC1 |
| | KFGSVRGNYLDIFNEKGHLLHTEKFPSPIVDVYLDNSLLAVDLEGVVREIDLSTHSSKEVATLQSLACAMFSKVDDKVTIAFKGSNSDFV | |
| | KIAILEDKVSTISTNISSVVHIKNNLLETDEGIYSIEGSTVKKILDGTAYLTDIGAISVDTVKNSVRSSGNSFEPQSKILKVHAEDEFIVVLT | |
| | VDEVLEIDLETFDLSSVKENSLTEEYLNSVDYEIFFKNQEVQLIIQDRSARELIITNGVIQKVLDLSLNDVVDYSIVTLQPQLKAIEDEIIEE | |
| | ENSTFFKAYTSRLFNTLAALKENIKKREFTSLFQYDTSGQDQSFGLDKRLVIGCSHGKLSAYHLLTKTPQLSWEIQLPLIDEVSSFNEGEVSV | |
| | LSGTTVFTIDAETGDILSETVATAEDPQKEFDIKSDDRTISGLKLINNEYSSTWTFKASPEEKILKVVRREDDNSNVASAGHILGNNSVLF | |
| | KYLFQNLISAVLLNEHTNDIRFVILNAITGQQVYSDVHSGIDSNTNVNLIYDENFIVVSYFGSDPIPEQHIVVYDLYESLTPNKRVEPKDG | |
| | LVSNFDTDTPIPQISSQSFLFPSRINFIAASRSKFGIASKWIISVLENGQIFAIPKVVLNSRRVVGRDLTSTEKQEYGMSVYSPFISLPENIF | |
| | TISNIRNLVLDNNSNTLPSGKPILTVEPTGLASTSFVCLINSFNVYCTQISPSKKFDMLRENFDQYKLLLSIFGLLAIVLLVRPYVYSRNVQK | |
| | LWTTKI* | |
| 35 | MLSLKPSWLTLAALLYAMLMVVVPFAKPVRADDVESYGTVIGIDLGTTYSCVGVMKSGRVEILANDQGNRITPSYVSFTEDERLVGD | BiP |
| | AAKNLAASNPKNTIFDIKRLIGMKFDSPEVQRDLKRLPYSVKSKNGQPIVSVEYKGEEKSFTPEEISAMVLGKMKLIAEDYLGKKVTHA | |
| | VVTVPAYFNDAQRQATKDAGLIAGLTVLRIVNEPTAAALAYGLDKTGEERQIIVYDLGGGTFDVSLLSIEGGAFEVLATAGDTHLGGE | |
| | DFDYRVVRHFVKIFKKKHNIDISDNDKALGKLKREVEKAKRTLSSQMTTRIEIDSFVDGIDFSEQLSRAKFEEINIELFKKTLKPVEQVLKD | |
| | AGVKKSEIDDIVLVGGSTRIPKVQQLLEDFFDGKKASKGINPDEAVAYGAAVQAGVLSGEEGVDDIVLLDVNPLTLGIETTGGVMTTL | |
| | NRNTAIPTKKSQIFSTAADNQPTVLIQVYEGERALAKDNNLLGKFELTGIPPAPRGTPQVEVTFVLDANGILKVSATDKGTGKSESITIN | |
| | NDRGRLSKEEVDRMVEEAEKYAAEDAALREKIEARNALENYAHSLRNQVTDDSETGLGSKLDEDDKETLTDAIKDTLEFLEDNFDTAT | |
| | KEELDEQREKLSKIAYPITSKLYGAPEGGAPPGQGFDDDDGDFDYDYDYDHDEL* | |
| 36 | MPIDIINTLVVKGTDGIPGWPIIKRYGLPFVALSLLKVYCGGKLNPWQRDVHGKVYILTGATAGVGSQLAEELAKGGAQLILLVKDPSS | YNL181W |
| | SWTVEFVDDLRERTGNPLVYAEQCDLADLHSVRKFATRWLDNTPPRRLDGIVGCAGEALPLGAARSTSSDGVERQVAVNYLGHFHL | |
| | LALLSPSLRAQPADRDVRVVLTTCTTQAMGQVSLDDPLWLDSQYPSKRPWQVFGGAKLMLGCFAQEFQRRLDATPRGDKMPSKL | |
| | RVNVVNPGFMRTASTARVLSFGSLWGLLLYLLLYPIWFILFKTPIQGAQSYLAALFAEHFIELPGGQFIQDCKIVKPARKELSDFTFQNK | |
| | LYEKTEKLIDQLERQSAKQRVRSKPKSNSKSKPSKKSGTANVGPEKENDVFASALKATPPDLFPHQRADPAGNKYLDQLEKKLAEQSK | |
| | KHST* | |
| 37 | MSFFSQLTGALDKPGFNWKLLIAGFSSAEFAFEAYLSYRQIKKLQEKGHQVPQSLKGKIEEDVALKSQDYSFTKLKFGIFSDAVNLLYNL | Ste24 |
| | TWIKFDILPKLWNLSGNLLANSLAFLPWKGTLVQSLVFVNLLSIAGLVVSLPLSYYSTFVIEEKFGFNKQTLKLWITDAIKGLLLSFVFGTA | |
| | IYAGFLKIVDYFSDTFMFYMSVFMFVIQIFFIIFYPKFIQPLFNKLTPLEDGELKQSIEKLAADQKFPLDKLYVIDGSKRSSHSNAYFLGLP | |
| | WGTKQIVIFDTLIEKSSVDEVTAVLGHEIGHWALSHTTKLLLINQVQLFSIFSLFALFFKNKSLYQSFGFSGQPVIIGFTLFSDVLKPFNAV | |
| | LSFATNLLSRNYEYQADEYAVDLGYSSDLSSALISLHKENLSSLHVDWLYSAYSHSHPHLTERLQAIEFNAKKEK* | |
| 38 | MSREDSVYLAKLAEQAERYEEMVENMKTVASSGLELSVEERNLLSVAYKNVIGARRASWRIVSSIEQKEEAKGNQSQVSLIREYRSKIE | Bmh2 |
| | TELANICEDILSVLSEHLIPSARTGESKVFYFKMKGDYHRYLAEFAVGDKRKEAANLSLEAYKSASDVAVTELPPTHPIRLGLALNFSVFY | |
| | YEILNSPDRACHLAKQAFDDAIAELETLSEESYKDSTLIMQLLRDNLTLWTSDMSETGQEESSNSQDKTEAAPKDEE* | |
| 39 | MRIVRSVAIAIACHCITALANPQIPFDGNYTEIIVPDTEVNIGQIVDINHEIKPKLVELVNTDFFKYYKLNLWKPCPFWNGDEGFCKYKD | Ero1 |
| | CSVDFITDWSQVPDIWQPDQLGKLGDNTVHKDKGQDENELSSNDYCALDKDDDEDLVYVNLIDNPERFTGYGGQQSESIWTAVYD | |
| | ENCFQPNEGSQLGQVEDLCLEKQIFYRLVSGLHSSISTHLTNEYLNLKNGAYEPNLKQFMIKVGYFTERIQNLHLNYVLVLKSLIKLQEY | |
| | NVIDNLPLDDSLKAGLSGLISQGAQGINQSSDDYLFNEKVLFQNDQNDDLKNEFRDKFRNVTRLMDCVHCERCKLWGKLQTTGYGT | |
| | ALKILFDLKNPNDSINLKRVELVALVNTFHRLSKSVESIENFEKLYKIQPPTQDRASASSESLGLFDNEDEQNLLNSFSVDQAVISSKEAP | |
| | EEIKSKPVGKAAYKQNSCPSLGSKSIKEAFHEELHAFIDAIGFILNSYRTLPKLLYTLFLVKSSELWDIFIGTQRHRDTTYRVDL* | |

### IV. EXAMPLES

The following examples are included for illustrative purposes only and are not intended to limit the scope of the invention.

### Example 1: Construction OVD Expression Strains Through Sequential Transformations

Expression cassettes for OVD were constructed using a library of promoters, signal sequences and terminators as follows.

Plasmid 1 was constructed with a pAOX1 promoter, an alpha mating factor secretion signal (seq 1; SEQ ID NO: 13) fused in frame with a cDNA encoding OVD (seq 1; SEQ ID NO: 13), followed by a tAOX1 terminator. Plasmid 1 also contained a Ura3 selection cassette on the backbone of the plasmid for selection.

Plasmid 2 was constructed with a pDAS2 (1) promoter, an alpha mating factor secretion signal (seq 1; SEQ ID NO: 13) fused in frame with a cDNA encoding OVD (seq 1; SEQ ID NO: 13), followed by a tAOX1 terminator. The backbone of Plasmid 2 did not contain a selection expression cassette.

Strain CF1 was constructed by transforming Pichia with a mixture of Plasmid 1 and Plasmid 2, colonies were identified that contained copies of both Plasmid 1 and Plasmid 2.

The selected strain CF1 was then transformed with Plasmid 3 to create strain CF2. Plasmid 3 included sequences for homologous integration into the AOX1 genomic site to create an AOX1 deletion phenotype. Following selection of a colony with the AOX1 deletion, as well as retaining the integrated Plasmids 1 and 2, this strain was then transiently transformed with a Cre recombinase to removed floxed sequences comprising plasmid backbones to create strain CF3.

For the transformations, at least 1 microgram of plasmid DNA was linearized with the restriction enzyme SmiI. The linearized DNA was ethanol precipitated after digestion and then resuspended in water.

Approximately 75 uL of prepared competent Pichia cells in 1M ice cold sorbitol were electroporated in 2 mm electroporation cuvettes (Bulldog Bio) using a Harvard Apparatus BTX ECM 630 electroporator device at 1500V 200 ohms with capacitor set to 25 μF. One mL of 1M sorbitol was added to the cuvette immediately after electroporation and the cells were then rested at 30° C. for 1 hour before plating on YPD agar plates with appropriate antibiotic (depending on the vectors chosen to match the antibiotic selection marker e.g., Zeocin, G418, Hygromycin or Nourseothricin) and then incubated at 30° C. for 3 days before identifying and picking colonies for assays.

The term “floxed” as used herein refers to the removal of DNA sequences from the genome, where the sequences were flanked on either side by a lox site. The expression of the recombinase excises the DNA sequences between the 2 loxP sites and the resulting strains therefore no longer carry the plasmid backbones. In the examples herein, two methods of recombinase expression were employed. In the first method, the Cre recombinase was expressed from a constitutive promoter on a replicating plasmid that did not integrate into the Pichia genome. The plasmid was maintained when antibiotic selection was maintained. After the plasmid backbones were removed by the recombinase, the resulting strains were removed from antibiotic selection and the recombinase plasmid was no longer maintained. Strains were identified that lost both the plasmid backbones and the recombinase expression plasmid. In the second method, the Cre recombinase was expressed from a methanol inducible promoter that was integrated as part of the plasmid backbone of one or more plasmid holding the expression cassette, where the backbone with the recombinase cassette were flanked on either side by loxP sides. Upon methanol induction, the recombinase was expressed which then excised this plasmid backbone, thus losing both the plasmid backbone and the recombinase cassette from the resulting strains.

Strain CF4 was created with strain CF3 as the starting material, then transformed with Plasmid 4. Plasmid 4 includes the pPEX11 promoter upstream of a sequence encoding a helper factor, followed by a tAOX1 terminator sequence. The backbone of Plasmid 4 includes a loxZeo SynUra selection cassette.

Additional copies of OVD were added to strain CF4 to create CF5 by transformation with Plasmid 5. Plasmid 5 contains a pAOX1 promoter upstream of an alpha mating factor secretion signal (seq 1; SEQ ID NO: 13) fused in frame to an OVD (seq 2; SEQ ID NO: 14) cDNA, followed by a tAOX1 terminator. The backbone also includes a CLPH selection cassette.

### Example 2: Construction of OVD Expression Strains Through Combination Transformations

A series of four plasmids were constructed (Plasmids 7-10), each containing alpha mating factor secretion signal (seq 1; SEQ ID NO: 13) fused in frame to an OVD (seq 2; SEQ ID NO: 14) encoding cDNA, followed by a tAOX1 terminator. Each plasmid contained a unique promoter upstream of the OVD fusion: Plasmid 7 contained pAOX1, Plasmid 8 contained pDAS2 (2), Plasmid 9 contained pFLD1 and Plasmid 10 contained pFDH1. Plasmids 7-10 each included 3 copies of the aforementioned OVD expression cassettes, and the backbone included a loxZeo selection cassette. Plasmids 7-10 were linearized prior to transformation as a mixture of all four plasmids into Pichia. The resulting selected strain was named CF6.

### Example 3: Comparison of Strains for Genome Stacking of Expression Constructs

Using the constructs and methods described in the previous examples, a number of strains were constructed as shown in the Table 7 below.

TABLE 7
Integration in strains

| | Animal Protein Gene (OVD) | | | Helper Factors (HAC1) | | |
|---|---|---|---|---|---|---|
| | Copy | | | Copy | | |
| | number | Promoters | Location | number | Promoters | Location |
| CF1 | 8 | Aox1, | Chr3 | 0 | 0 | NA |
| CF2 | | DAS1 | | | | |
| CF3 | | | | | | |
| CF4 | 8 | Aox1, | Chr3 | 2 | Pex11 | Teleomere |
| | | DAS1 | | | | |
| CF5 | 8 | Aox1, | Chr3 | 2 | Pex11 | Teleomere |
| | | DAS1 | | | | |
| CF6 | 15 | AOX1, | Chr4 | 0 | 0 | NA |
| | | DAS, | | | | |
| | | FDH1, | | | | |
| | | FLD1 | | | | |

### Example 4: Analysis of Protein Expression

Colonies from transformations were picked into a 96-deep-well plate in YPD using a Qpix colony picker. The picked colonies were grown at 30° C. in a plate shaker for 24 hours before being spun down, old media removed, and new induction media containing glucose and methanol was added. The induction phase was continued for 96 hours with daily feeding of the glucose/methanol mixture.

To assay for secreted protein expression, the cells and media were centrifuged and an aliquot of the resulting supernatant was assayed for protein content. The supernatant was added directly to the protein assay reagent (Coomassie Plus Protein Assay Reagent from Thermo Scientific) in some cases, the supernatant was diluted in 100 mM potassium phosphate buffer prior to adding it to the protein assay reagent. Samples were incubated with the protein assay reagent for 10 minutes and then read at 595 nm wavelength using a Spectra Max M2 plate reader (Molecular Devices). The data were calculated as grams per liter of the protein.

To assay for protein quality and confirm the results of the protein assays, the supernatants were analyzed by SDS PAGE and stained with Simply Blue Safe Stain (Life Technologies). The resulting gel images were documented using a Protein Simple Imager.

### Example 5: Protein Expression Levels for OVD Strains

Protein expression of four genome stacked OVD strains, CF3, CF4, CF10 and CF6, were compared to each other in a high throughput screen (HTS) using a deep well plate format for growth (as described in Example 4 and in a 2-liter bioreactor under high cell density growth conditions (DASGIP) for total protein (as further explained in Example 7). Strains CF10 (105.3%) and CF6 (100%) showed higher total protein expression compared to CF3 (74.9%) and CF4 (76.5%) strains in the HTS deep well plate format for growth using the CF6 produced protein titer as a baseline (100%). In the DASGIP conditions, using the CF6 produced protein titer as a baseline (100%), CF4 and CF10 had total large scale protein production of approximately 164% and 200% respectively, as compared to the CF3 (˜135%) and CF6 strains.

### Example 6: Expression in High-Volume Fermentation Conditions

Strains CF3, CF5 and CF10 were grown in 40-liter fermentation tanks. Yeast strain glycerol stocks were thawed and inoculated at a 0.2% inoculum ratio in baffled shake flasks containing BMDY media (BMDY media is similar to BMGY media, with the glycerol, ‘G’, having been replaced with glucose/dextrose, ‘D’, Pichia Easy Select Manual, Thermo Fisher). Shake flasks were incubated at 30° C. and 250 rpm for 26 hrs. Shake flask cultures were then transferred at a 10% ratio to bioreactors containing BSM (basal salt medium), glucose, and trace metals Pichia Fermentation Process Guidelines, Thermo Fisher).

The bioreactor fermentation was divided into three phases. During phase 1, the culture was grown for 24 hours until all glucose was consumed. During phase 2, the culture was fed glucose at a glucose-limiting rate for 12 hours. Finally, in phase 3, the culture was induced by continuously feeding a co-feed of glucose and an activator of an inducible promoter, i.e., methanol for 96 hours.

Expression levels of OVD in the supernatant were measured as g/liter of media. Table 8 shows the relative protein expression of OVD in the supernatant in the different strains grown in a bioreactor. The titers were measured as g/L and used to calculate fold improvement or presented at relative levels in Table 8. Strains CF10 was the highest expressors of secreted OVD.

TABLE 8
Relative Expression Levels

| Strain | Relative Expression Levels |
|---|---|
| CF3 | +++ |
| CF5 | ++ |
| CF10 | ++++ |

### Example 7: Comparison of OVD Engineered Strains with Homologous Versus Non-Homologous Integration Sites

A series of transformants comprising AOX1 expression cassettes were generated by integrating the cassettes by homologous recombination just upstream of the AOX1 gene (i.e., relying on the sequence homology between the AOX1 promoter in the cassettes and the genomic AOX1 sequence). The number of copies of OVD in each strain as determined by sequencing were approximately 5 in the CF11 strain, 1 in the CF12 strain and 7-8 in the CF13 strain. The number of copies of OVD in these strains correlated with the level of expression of OVD. These 3 engineered strains were compared with CF1 (Example 3) which has only 4-5 copies of OVD integrated at a non-homologous site. Surprisingly, none of the homologously integrated engineered OVD strains had expression levels comparable to CF1; the CF1 strain was significantly higher in OVD expression than CF11 which had similar copy number of OVD and significantly higher than CF13 which has a higher copy number of OVD.

In a separate experiment, two Pichia strains CF14 and CF15, which already expressed OVD, were further transformed with additional copies of OVD using plasmids. Both CF14 and CF15 were derived from CF16, a derivative strain of CF6 described in Example 2 and comprised an added Hac1 helper factor gene driven by pPEX11. CF14 and CF15 were transformed with 2 plasmids, Plasmid 3× containing 3 copies of OVD, Plasmid 6×containing 6 copies of OVD. The OVD copies in the 3× and 6× plasmids were driven by AOX1, DAS and FLD promoters. The AOX1 terminator was used in both plasmids. Between 80-320 transformants were selected for each set (as shown in Table 9). High expressors were selected from each set. PCR was used to confirm the site of integration of the OVD plasmids. Using the CF6 produced protein titer as a baseline (100%), DASGIP titers for CF14 and CF15 were 176% and 164%.

Six of the CF15 retransformed sets and three of the CF14 retransformed sets (Plasmid 3× and 6× transformants) were streaked out for single colonies and 5 or 6 single colonies were picked and assayed for high protein expression. Table 9 indicates the number of transformants screened for each transformation. Table 10 indicates the number of transformants that were positive for insertion at the desired site compared to the total number of transformants screened when tested by PCR.

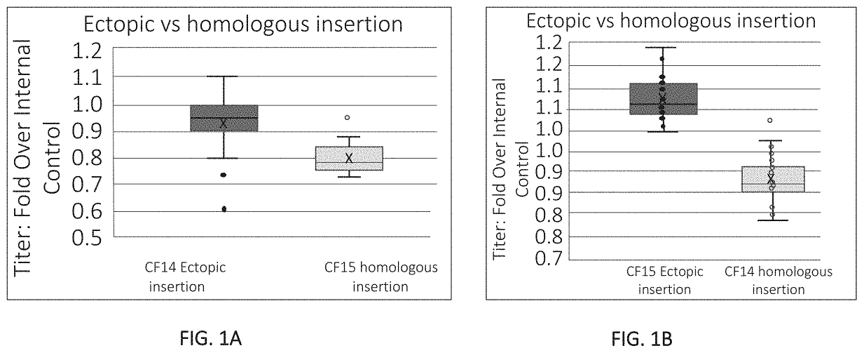

FIGS. 1A-B show a summary of CF14 and CF15 rescreens from this experiment separating the re-transformant sets by homologous and non-homologous (ectopic) integration sites of the Plasmid 3× and 6×.

The ectopically integrated (i.e., integrated at a non-homologous genomic site) transformants produced the highest expression in each re-transformant set (re-transformants from the 3× and 6× plasmids were not distinguished in this data).

TABLE 9
Transformants

| | CF14 | CF15 |
|---|---|---|
| 3x | 240 | 160 |
| 6x | 320 | 80 |

TABLE 10
Transformants positive for insertion at the desired site

| | CF14 | CF15 |
|---|---|---|
| 3x | 3/8 | 4/6 |
| 6x | 4/8 | 2/3 |

### Example 8: Comparison of OVD Strains

Strains D1-D10 of Pichia pastoris were made similar to the methods described in Examples 1 through 7. Some of the strains comprised a helper factor HAC1. The expression cassette for HAC1 expression used the PEX11 promoter and AOX1 terminator sequence for expression in all cases, except D10 where DAS promoter was used in addition to PEX11 for HAC1 expression. Table 11 below shows the promoters present in the sequence for each strain, the copy numbers for OVD and the copy number for the helper factor. Results are provided below in Table 11. Table 11 demonstrates substantially improved titers when using helper copies, even when reducing the number of heterologous gene copies. As demonstrated, reducing the number of heterologous gene (OVD) copies from 15 to 12, while concurrently adding 2 helper copies, increased deep well (about 10% on average) and DASGIP titers (about 50% on average). Further increasing the helper copy number to 4 had mixed results, however (decreasing deep well titer, increasing DASGIP titer).

TABLE 11
Results for OVD strains

| | | | | Small scale HTS | DASGIP Titer |
|---|---|---|---|---|---|
| | | OVD | Helper | Titer using CF6 | using CF6 titers |
| Strain | OVD | Copy | Copy | titers as baseline | as baseline |
| number | Promoters | numbers | Number | (100%) | (100%) |
| D1 | AOX1, | 15 | 0 | 105.6% | 97% |
| | FDH1, | | | | |
| | FLD1, | | | | |
| | DAS1 | | | | |
| D2 | AOX1, | 15 | 0 | 129.4% | 94% |
| | FDH1, | | | | |
| | FLD1, | | | | |
| | DAS1 | | | | |
| D3 | AOX1, | 15 | 0 | 127.9% | 135% |
| | FDH1, | | | | |
| | FLD1, | | | | |
| | DAS1 | | | | |
| D4 | AOX1, | 12 | 2 | 149.5% | 159% |
| | FDH1, | | | | |
| | FLD1, | | | | |
| | DAS1 | | | | |
| D5 | AOX1, | 12 | 2 | 146.4% | 162% |
| | FDH1, | | | | |
| | FLD1, | | | | |
| | DAS1 | | | | |
| D6 | AOX1, | 12 | 2 | 97.5% | 178% |
| | FDH1, | | | | |
| | FLD1, | | | | |
| | DAS1 | | | | |
| D7 | AOX1, | 11 | 2 | 143.7% | NA |
| | FDH1, | | | | |
| | FLD1, | | | | |
| | DAS1 | | | | |
| D8* | AOX1, | 12 | 2 | 120.7% | 153% |
| | FDH1, | | | | |
| | FLD1, | | | | |
| | DAS1 | | | | |
| D9* | AOX1, | 12 | 2 | 142.4% | 179% |
| | FDH1, | | | | |
| | FLD1, | | | | |
| | DAS1 | | | | |
| D10* | AOX1, | 11 | 4 | 107.1% | 207% |
| | FDH1, | | | | |
| | FLD1, | | | | |
| | DAS1 | | | | |

The strains with asterisks were further modified to have reduced methanol utilization capability which simplifies fermentation conditions.

### Example 9: Construction of Pepsinogen Expression Strains Through Combination Transformations

A series of plasmids containing expression cassettes for pepsinogen were constructed as follows. All plasmids contained a loxZeo selectable marker cassette in its backbone and all plasmids were linearized prior to transformation into Pichia. Plasmid 11 contained 3 head-to-tail copies of a cassette with a pAOX1 promoter, an alpha mating factor secretion signal fused in frame with a pepsinogen (PGA) cDNA (SEQ ID NO: 15), followed by a tAOX1 terminator.

Plasmid 12 was constructed with 2 head-to-tail copies of a cassette with a pAOX1 promoter, an alpha mating factor secretion signal fused in frame with a PGA cDNA, followed by a tAOX1 terminator, and 1 copy of a cassette with a pFDH1 promoter, an alpha mating factor secretion signal fused in frame with a PGA cDNA, followed by a tAOX1 terminator.

Plasmid 13 was constructed with 2 head-to-tail copies of a cassette with a pAOX1 promoter, an alpha mating factor secretion signal fused in frame with a PGA cDNA, followed by a tAOX1 terminator, and 1 copy of a cassette with a pFLD1 promoter, an alpha mating factor secretion signal fused in frame with a PGA cDNA, followed by a tAOX1 terminator.

Plasmid 14 contained 2 head-to-tail copies of a cassette with a pAOX1 promoter, an alpha mating factor secretion signal fused in frame with a PGA cDNA, followed by a tAOX1 terminator, and 1 copy of a cassette with a pDAS2 (3) promoter, an alpha mating factor secretion signal fused in frame with a PGA cDNA, followed by a tAOX1 terminator. Plasmid 14 was constructed to have 4 head to tail copies of a cassette with a pAOX1 promoter, an alpha mating factor secretion signal fused in frame with a PGA cDNA, followed by a tAOX1 terminator.

Plasmid 15 contained 2 head-to-tail copies of a cassette with a pAOX1 promoter, an alpha mating factor secretion signal fused in frame with a PGA cDNA, followed by a tAOX1 terminator and 2 copies of a cassette with a pFLD1 promoter, an alpha mating factor secretion signal fused in frame with a PGA cDNA, followed by a tAOX1 terminator.

Plasmids 11-15, all linearized, were combined in a starting mixture of nucleic acids for a single transformation reaction into Pichia. Strain CF9 was isolated from the transformation.

### Example 10: Construction of Pepsinogen Expression Strains Through Sequential Transformations

Strain CF9 was used as the starting material. It was then transformed with Plasmid 6 and Plasmid 16, which contained a pAOX1 promoter, an alpha mating factor secretion signal fused in frame with a PGA cDNA, followed by a tAOX1 terminator with the backbone containing a CLPH marker cassette. The floxed backbones of the transformations were removed. Strains CF7 and CF8 were isolated from these sequential transformations.

### Example 11: Transformation of P. pastoris for Genome Stacking with Pepsinogen Expression Cassettes

The P. pastoris strain BG08 (BioGrammatics Inc., Carlsbad; CA, USA) was a single colony isolate from the Phillips Petroleum strain NRRL Y-11430 obtained from the Agriculture Research Service culture collection (Sturmberger, et al. 2016). P. pastoris BG10 (BioGrammatics Inc, Carlsbad, Calif., USA) was derived from BG08 using Hoechst dye selection to remove cytoplasmic killer plasmids (Sturmberger, et al. 2016). The resulting BG10 strain was then further modified to have a deletion in the Alcohol Oxidase 1 gene (AOX1). This deletion generates a methanol-utilization slow phenotype that reduces the strain's ability to consume methanol. This base strain was called DFB-001 and used for the transformation of the pepsinogen construct.

The pepsinogen construct, along with a construct for the expression of the P. pastoris transcription factor HAC1 under the control of a strong methanol inducible promoter, was transformed into Pichia pastoris and isolates were selected that expressed and secreted pepsinogen. A transformant was selected as a high-producer for use in subsequent steps. Propagation of the high-producer strain confirmed that all changes introduced into the strain were stably integrated in the genome and confirmed to be present after >45 generations of growth on non-selective growth media.

The selected transformant (resulting strain) was DNA sequenced; it contained three copies of the HAC1 expression cassette and five copies of the pepsinogen expression cassette. Sequencing also confirmed that this strain did not contain any antibiotic markers or prokaryotic vector origin of replication sequences. Sequencing showed that the pepsinogen cassettes were all located together at a locus on chromosome 1 of the resulting strain (see Table 12 below).

TABLE 12
Integration in strains

| | Animal Protein Gene (PGA) | | | Helper Factors | | |
|---|---|---|---|---|---|---|
| CF7 | 2, 1 | AOX1, | Chr1 | 1 | Pex11 | Chr1 |
| | | FDH1 | | | | |
| CF8 | 5 | Aox1, | Chr1 | 3 | Pex11 | Chr2 |
| | | FDH1 | | | | |

### Example 12: Protein Expression Levels for Pepsinogen Strains

Protein expression of two pepsinogen genome stacked strains, CF7 and CF8, were compared to each other under two growth conditions. The first was expression in a high throughput screen (HTS) using a deep well plate format for growth as described in Example 3. The strain CF8 showed higher protein expression than the CF7 strain. The strains were also compared in a 2-liter bioreactor under high cell density growth conditions (DASGIP, see Example 7). Briefly, the yeast strain glycerol stocks were thawed and inoculated at a 0.2% inoculum ratio in baffled shake flasks containing BMDY media (BMDY media is similar to BMGY media, with the glycerol, ‘G’, having been replaced with glucose/dextrose, ‘D’, Pichia Easy Select Manual, Thermo Fisher). Shake flasks were left to incubate at 30° C. and 250 rpm for 26 hrs. Shake flask cultures were then transferred at a 10% ratio to bioreactors containing BSM (basal salt medium), glucose, and trace metals Pichia Fermentation Process Guidelines, Thermo Fisher). The bioreactor fermentation was divided into three phases. During phase 1, the culture was grown for 24 hours until all glucose was consumed. During phase 2, the culture was fed glucose at a glucose-limiting rate for 12 hours. Finally, in phase 3, the culture was induced by continuously feeding a co-feed of glucose and an activator of an inducible promoter, i.e., methanol for 96 hours.

The strains were assayed for total secreted protein and then for total secreted protein of interest (assayed as present in the supernatant). In both measurements, the CF8 strain outperformed the CF7 strain. Using P5 small scale titers from Example 13 below, the small scale HTS titer for CF7 was 22% and 45% for CF8. Using P5 large scale titers from Example 13 below, the large scale DASGIP titer for CF7 was 108% and 117.5% for CF8.

### Example 13: Comparison of Pepsinogen Strains

Strains P1-P5 of Pichia pastoris were made similar to the methods described in Examples 9 through 12 where all expression cassettes used AOX1 terminator sequences. Some of the strains comprised a helper factor HAC1. The expression cassette for HAC1 expression used the PEX11 promoter and AOX1 terminator sequence for expression in all cases. Table 13 below shows the promoters present in the sequence for each strain, the copy numbers for PGA and the copy number for the helper factor. Results are provided below in Table 13.

TABLE 13
Results for PGA strains

| | | | | Small-scale | DASGIP |
|---|---|---|---|---|---|
| | PGA | | Helper | titer using P5 | Titer using |
| Strain | Copy | Promoters | Copy | titer as a | P5 titer as |
| number | Number | PGA | Number | baseline | a baseline |
| P1 | 5 | FDH1, | 1 | 31.3% | 128.42% |
| | | AOX1 | | | |
| P2 | 7 | FDH1, | 4 | 66.8% | 124.04% |
| | | AOX1 | | | |
| P3 | 9 | FDH1, | 4 | 59.9% | 125.68% |
| | | AOX1 | | | |
| P4 | 3 | AOX1, | 1 | 7.9% | 109.29% |
| | | FDH1 | | | |
| P5 | 8 | Aox1, | 4 | 100% | 100.00% |
| | | FDH1 | | | |

### Example 14: Comparison of Ovalbumin Strains

Strains V1-V8 expressing ovalbumin (OVA) of Pichia pastoris were made similar to the methods described in for OVD and PGA where all expression cassettes used AOX1 terminator sequences. SEQ ID NO: 16 was operably linked to promoters described below in Table 14. Strain V1 was the base strain, strain V2 was transformed with expression cassettes with promoters FLD and DAS1 driving the expression of OVA. Strain V2 was transformed with additional expression cassettes, with AOX1 promoter driving the expression of OVA resulting in strain V7. Some of the strains (such as V7) comprised a helper factor HAC1. The expression cassette for HAC1 expression used the Pex11 promoter and AOX1 terminator sequence for expression in all cases. Table 14 below shows the promoters present in the sequence for each strain, the copy numbers for OVA and the copy number for the helper factor.

Table 14 demonstrates substantially improved titers when using helper copies, particularly in certain ratios. As demonstrated, use of HAC1 copies substantially improved both deep well and DASGIP titers. As can be seen, increasing the number of heterologous gene (OVA) copies did little to improve the titers (compare 3 copies of OVA vs 9 copies of OVA), but addition of HAC1 copies showed a dramatic improvement in titers, with over 5 fold improvements in deep well titer and up to 3 fold or more improvement in DASGIP titer. Further, as with the diminishing returns observed for increased number of heterologous gene copies, diminishing (or reduced) returns were observed when increasing the number of HAC1 copies from 2 to 5, suggesting optimum working ratios of heterologous gene copies to helper copies.

TABLE 14
Results for OVA strains

| | | | | Small-scale | DASGIP |
|---|---|---|---|---|---|
| | OVA | | HAC1 | titer using V2 | Titer using |
| Strain | Copy | OVA | Copy | titer as a | V2 titer as |
| number | Number | Promoters | Number | baseline | a baseline |
| V1 | 0 | NA | NA | | 0 |
| V2 | 3 | FLD, DAS | 0 | 100% | 100% |
| V3 | 3 | FLD, DAS | 0 | 140.5% | — |
| V4 | 9 | AOX1, FLD, | 0 | 154.5% | — |
| | | DAS1 | | | |
| V5 | 10 | AOX1, FLD, | 0 | 179.5% | — |
| | | DAS1 | | | |
| V6 | 16 | AOX1, FLD, | 5 | 1136.4% | 153.5% |
| | | DAS1 | | | |
| V7 | 16 | AOX1, FLD, | 5 | 981.8% | 216.3% |
| | | DAS1 | | | |
| V8* | 16 | AOX1, FLD, | 2 | N/A | 374.4% |
| | | DAS1 | | | |

The strains with asterisks were further modified to have reduced methanol utilization capability which simplifies fermentation conditions

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An engineered host cell for expressing a heterologous protein, said engineering host cell comprising at least three different expression cassettes integrated into the genome of the engineered host cell wherein;
a. a first expression cassette comprising a first promoter operably linked to a heterologous gene sequence encoding the heterologous protein
b. a second expression cassette comprising a second promoter operably linked to a heterologous gene sequence encoding the heterologous protein;
c. a third expression cassette comprising a third promoter operably linked to a helper factor sequence; and
d. a copy number ratio of the helper factor encoding sequence to the heterologous protein encoding sequence is at least 1:10.

2.-95. (canceled)

96. The engineered host cell of claim 1, the copy number ratio of the helper factor encoding sequence to the heterologous protein encoding sequence is at most 1:2.

97. The engineered host cell of claim 1, wherein the copy number ratio of the helper factor encoding sequence to the heterologous protein encoding sequence is at least 1:9, 1:8, 1:7, 1:6, 1:5, 1:4 or 1:3.

98. The engineered host cell of claim 1, wherein the copy number ratio of the helper factor encoding sequence to the heterologous protein encoding sequence is at most 1:9, 1:8, 1:7, 1:6, 1:5, 1:4, 1:3 or 1:2.

99. The engineered host cell of claim 1, wherein at least one promoter is an inducible promoter.

100. The engineered host cell of claim 99, wherein the inducible promoter is a methanol inducible promoter.

101. The engineered host cell of any one of claim 1, wherein at least one promoter is a constitutive promoter.

102. The engineered host cell of claim 1, wherein the host cell comprises at least 2 copies of the first expression cassette.

103. The engineered host cell of claim 1, wherein the host cell comprises at least 2 copies of the second expression cassette.

104. The engineered host cell of claim 1, wherein the host cell comprises at least 1 copy of a fourth expression cassette comprising a fourth promoter operably linked to the heterologous gene sequence.

105. The engineered host cell of claim 1, wherein the first cassette and the second cassette are integrated into the genome in the same 5′ to 3′ orientation.

106. The engineered host cell of claim 1, wherein the first cassette and the second cassette are integrated into the genome in an opposite 5′ to 3′ orientation.

107. The engineered host cell of claim 1, wherein the host cell comprises at least 2 copies of the helper factor encoding sequence in 1 or 2 expression cassettes.

108. The engineered host cell of claim 1, wherein the heterologous protein is a food-related protein.

109. The engineered host cell of claim 108, wherein the food-related protein comprises an enzyme, a nutritive protein, a food ingredient or a food additive.

110. The engineered host cell of claim 109, wherein the food-related protein comprises an egg-white protein.

111. The engineered host cell of claim 110, wherein the egg-white protein is ovomucoid.

112. The engineered host cell of claim 111, wherein a copy number ratio of the helper factor encoding sequence to ovomucoid encoding sequence is from 1:3 to 1:6.

113. The engineered host cell of claim 110, wherein the egg-white protein is ovalbumin.

114. The engineered host cell of claim 113, wherein a copy number ratio of the helper factor encoding sequence to ovalbumin encoding sequence is from 1:3 to 1:8.

115. The engineered host cell of claim 1, wherein the engineered host cell is capable of producing at least about 5 g per liter of the heterologous protein under fermentation conditions.

116. The engineered host cell of claim 1, wherein each of the helper factor gene sequences encodes for a protein independently selected from the group consisting of HAC1, Serine/threonine protein kinase 2 (Kin2), squalene synthase (ERG9), protein disulfide isomerase 1 (PDI1), SSA1, SSA4, SSB1, SSE1, BiP, ER Membrane Protein Complex Subunit 1 (EMC1), YNL181W oxidoreductase, integral membrane protein zinc metalloprotease Ste24, 14-3-3 protein Bmh2 and ER oxidoreductin 1 (Ero1).

117. The engineered host cell of claim 1, wherein the host cell is engineered to favor non-homologous integration over homologous integration and/or the host cell is selected based on a greater number of non-homologous integrations than homologous integrations.

118. The engineered host cell of claim 1, wherein at least two of the expression cassettes comprising the heterologous gene sequence integrate at different integration sites.

119. The engineered host cell of claim 1, wherein the host cell is a yeast cell.


SEQUENCE LISTING (39 sequences)

SEQ ID NO: 1 - DNA, 930 bp, synthetic construct
  misc_feature 1..930: note = Synthetic Polymer
  source 1..930: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaa

SEQ ID NO: 2 - DNA, 1051 bp, synthetic construct
  misc_feature 1..1051: note = Synthetic Polymer
  source 1..1051: mol_type = other DNA
aaatctgagacaacgatgaacctcccatgtagattccaccgccccagttacttttttgggcaatcctgttgataagatccattttagagttgtttcatgaaaggattacaggcgttgaagggtcagagagatgccagagaacagaccaattggtagtttgctaaagtggacgtctggcaggtgctctatcgtgttctttatttagggcgttacacttagtaggattacgtaacaatttggcttaaccttctaagttagaaagaaaccaagaggggtcctctttaacgttcagcagtatctaaaacacaaaacctgccctcataatacatcattctatctgtcaagctgtgctaccccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaa

SEQ ID NO: 3 - DNA, 1048 bp, synthetic construct
  misc_feature 1..1048: note = Synthetic Polymer
  source 1..1048: mol_type = other DNA
gatctctgagacaacgatgaacctcccatgtagattccaccgccccaattactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaagg

SEQ ID NO: 4 - DNA, 564 bp, synthetic construct
  misc_feature 1..564: note = Synthetic Polymer
  source 1..564: mol_type = other DNA
tgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagacttctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaa

SEQ ID NO: 5 - DNA, 750 bp, synthetic construct
  misc_feature 1..750: note = Synthetic Polymer
  source 1..750: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatc

SEQ ID NO: 6 - DNA, 1005 bp, synthetic construct
  misc_feature 1..1005: note = Synthetic Polymer
  source 1..1005: mol_type = other DNA
aaataaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacag

SEQ ID NO: 7 - DNA, 1002 bp, synthetic construct
  misc_feature 1..1002: note = Synthetic Polymer
  source 1..1002: mol_type = other DNA
cagccattaatctcacctcagtttttgaatcagtagaatttttaatgaaacaaacggttggtatattatttgatagagttgccaaatttccaaagataaatttttcatcaggtaatatcctgaataccgtaacatagtgactattggaagacactgctatcatattatatttcggataaaaatccaaaccccagaccgacctcttgagtctcaactccaagtcagccgcaactttaattatccgtggattgggagctagtttggacaacgcatcagtataatataactttacggttccattatcagacgctattgcaagaacttcctttccattgatctcgccaatgcggcagtaattgatatcgtagggtaggtctggaaagacgctggcgcttgtgtcccattctgcaggaatctctggcacggtgctaatggtagttatccaacggagctgaggtagtcgatatatctggatatgccgcctataggataaaaacaggagagggtgaaccttgcttatggctactagattgttcttgtactctgaattctcattatgggaaactaaactaatctcatctgtgtgttgcagtactattgaatcgttgtagtatctacctggagggcattccatgaattagtgagataacagagttgggtaactagagagaataatagacgtatgcatgattactacacaacggatgtcgcactctttccttagttaaaactatcatccaatcacaagatgcgggctggaaagacttgctcccgaaggataatcttctgcttctatctcccttcctcatatggtttcgcagggctcatgccccttcttccttcgaactgcccgatgaggaagtccttagcctatcaaagaattcgggaccatcatcgatttttagagccttacctgatcgcaatcaggatttcactactcatataaatacatcgctcaaagctccaactttgcttgttcatacaattcttgatattcacagg

SEQ ID NO: 8 - DNA, 619 bp, synthetic construct
  misc_feature 1..619: note = Synthetic Polymer
  source 1..619: mol_type = other DNA
cttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaacc

SEQ ID NO: 9 - DNA, 261 bp, synthetic construct
  misc_feature 1..261: note = Synthetic Polymer
  source 1..261: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcg

SEQ ID NO: 10 - DNA, 476 bp, synthetic construct
  misc_feature 1..476: note = Synthetic Polymer
  source 1..476: mol_type = other DNA
aattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgca

SEQ ID NO: 11 - DNA, 267 bp, synthetic construct
  misc_feature 1..267: note = Synthetic Polymer
  source 1..267: mol_type = other DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagct

SEQ ID NO: 12 - DNA, 267 bp, synthetic construct
  misc_feature 1..267: note = Synthetic Polymer
  source 1..267: mol_type = other DNA
atgagattcccttctattttcactgctgttttgttcgctgcttcttctgctttggctgctccagttaacactactaccgaagacgaaactgctcaaattcctgctgaagctgttattggttactctgacttggaaggtgacttcgacgttgctgttttgccattctctaactctactaacaacggtttgttgttcattaacactactattgcttctattgctgctaaggaagaaggtgtttctttggacaagagagaagctgaagct

SEQ ID NO: 13 - DNA, 561 bp, synthetic construct
  misc_feature 1..561: note = Synthetic Polymer
  source 1..561: mol_type = other DNA
gctgaagtagactgctcaagatttccaaatgctactgacaaggaaggaaaggatgtcctcgtatgtaacaaggaccttagacccatttgcggtacggatggcgtgacatacactaatgattgtttactatgtgcctatagcattgagttcggtacaaacatctccaaagagcacgatggagaatgtaaagagactgtccctatgaactgttcctcttacgcaaatacaacttcagaggacggtaaggtgatggtcttgtgtaacagggctttcaatccagtttgtggtactgacggtgttacttacgataacgaatgtctgttgtgtgctcataaagttgagcaaggagcatctgttgataaaagacacgatggtggatgccgtaaggaattggccgcagtttcggtggactgctccgaatatccaaaacctgactgtaccgctgaggatcgtcctctgtgcggaagtgacaacaagacctatggtaataagtgtaatttctgtaatgctgttgttgaaagcaatggtacattaacattgtctcattttggtaaatgttaa

SEQ ID NO: 14 - DNA, 558 bp, synthetic construct
  misc_feature 1..558: note = Synthetic Polymer
  source 1..558: mol_type = other DNA
gcagaagttgactgttctcgtttcccaaatgctactgacaaggaaggaaaagacgtcttggtgtgtaacaaggatttgaggccaatttgtggtacagatggtgtgacttacactaatgattgtctactttgcgcatatagcatcgagtttggaaccaatatctcaaaagagcacgacggtgaatgtaaagagactgtcccaatgaactgttcttcctacgctaatacaacctccgaggatggtaaagtaatggttttgtgcaacagagcctttaatcctgtttgtggcacggatggagtcacttatgataatgaatgtctcctgtgcgcccacaaggtagaacaaggtgctagcgttgataagcgtcatgacggtggatgtagaaaggaattagctgctgtgtctgttgattgttcagaatatcccaagcctgactgtacagctgaggacagacctctgtgcggttccgacaacaaaacatacggaaacaaatgcaacttctgtaatgcagtggttgagtcgaatggaacattgactttaagtcatttcggtaaatgt

SEQ ID NO: 15 - DNA, 1113 bp, synthetic construct
  misc_feature 1..1113: note = Synthetic Polymer
  source 1..1113: mol_type = other DNA
ctagtaaaggtgcctctagttagaaagaagagtctgagacaaaacctaattaagaacggaaaactgaaggatttcttaaaaacgcataaacataaccccgcctccaaatactttcctgaagcagccgctttaataggcgacgaacctttagaaaattacttagataccgagtatttcggcactattggtattggtacgcccgcacaagatttcacggtaatcttcgacaccggcagttcaaatttatgggtgccctccgtgtattgtagtagtttggcttgctccgaccataatcagttcaaccccgatgattcctccacgttcgaggccacgagtcaagaattgagtataacctacggcaccggttccatgacaggcatcctaggatacgatacagtacaagtcggcggcatttccgacaccaatcagatatttggcctaagtgagaccgagcccggatctttcttgtactacgcccctttcgacggaatcttgggtctagcttatcctagtatatctgcatccggagctacacccgtgtttgacaacctatgggatcagggccttgtctcccaggatctattctcagtctacctgagtagtaatgatgattcaggctcagtagtgttgctaggcggaattgattctagttactacacaggttctctgaactgggttcctgtcagtgtagagggctattggcagatcacactggattccataactatggatggagagaccatcgcctgctccggcggttgtcaggcaatagtggataccggaaccagtctgttgactggccctacctctgccatagctaatatacaaagtgatataggagcatctgagaactctgacggcgagatggtaatctcttgttctagtatcgattcattacctgacatagtttttaccataaatggtgttcaataccccctaagtccttccgcctatatcttgcaagatgatgactcatgtacaagtggctttgaaggtatggatgtacccacgtcatcaggtgagctttggatactgggcgatgtgtttatcaggcaatactacaccgtgttcgatagggctaacaacaaggtgggtctagcacctgttgcataa

SEQ ID NO: 16 - protein, 474 aa, synthetic construct
  REGION 1..474: note = Synthetic Polymer
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 17 - DNA, 1885 bp, synthetic construct
  misc_feature 1..1885: note = Synthetic Polymer
  source 1..1885: mol_type = other DNA
agtgaaaacgaaaagtgaaaatatcctgaggacgacttttattcttttggctggtgctagcgctgcatgttcgttactagccgttcaatacccattttctaaagttcagtcaatacatttagcaaggttggaagcgttggatattttcaacgagaactcctgggataagaaaagtaaattccgtctatattaccgatcatacttagatacattccaccagttggtgagcttacatgagaagtctaaactatcttggacccgctggttttataaagggttcgttaggaatgcgttaactaccattccagcaacatccgtggggcttctggtgtttgaaatactgcgtcaaaaattgagcgatgaaattgaagatcgattcagttgaatcgcccgaaacaattgatcccctgtacatacttgtaatttacctcagaatattttggtaagcttcccacccagcttttctataccgttcaccttcttttaagggatctctgcccttgccaaacaaaccacgacctacgattatgatgtcagtgccagtggaaaatacttgactcactgttcgatattgttggcctagagcatcaccagtgtcatccaaaccaacacctggtgtcataataatccaatcgaacccttcatcttgtcctcccatagaattttgagcaataaacccaatgacgaattccttgtctgattttgcaatttctacagtttcttcggtgtacttaccatgggcaattgatccctttgacgacagttcagccaacatcaatagtccccttggttgatctgttgtctcagtggctgcctcctttagaccctttacaattccactaccaatgacaccatgagcatttgtaatatctgcccattgtgcaatcttgtagacacctccttgatattgatgcttgacagtgttgcctatatcagcaaactttctgtcctcaaaaattaaaaacttgtgtttctttgatagttccaataaaggcagaatagttccatcatacgtgaagtcatcaattatgtcgatatgagtcttggccaaacagataaatgggcccaatttatctagaagctccaataattctttagttgttctcacgtcgactgatgcgcataggttactctgtttctgttccataagcgcaaacagtcgtcgtgccacaggtgattgatgagtatttgctctctcggcataactgcgagccattgtctaggtatctatccctttgatcaggttgatgttaactcattagagtggatcaatgcgaaggataggtgcgacgtgtaccgtccaaaaaacttttttcttcaatcttgacaaaaactggtaacagagagagcaagtgctaactctaccccaaccaagtacatcacaaaatggacgcattgaacgctaaagaacaacaagagttccagaaactcgttgaacaaaaacaaatgaaagacttcatgcgtctttactccgatttggttagcaaatgttttacagactgtgtcaatgattttacatctaacaagttgacttctaaggaggaaggctgcatcaacaagtgtgcagaaaagttcctcaagcacagtgagagagttggtcaacgtttccaagaacaaaaccaacttatgatgcaacagctaagacgttaaccccatatttttgtacataaagttcattgtccaggactaatccagactttctctgaacagctctataatcttagtagtttcttccatcatttcaatcgttagcttcgaaacatcactgtcttcatcgttatagatgatggcatttgtaaacatgatctgtagaactttagtcaattcgtcaaaggtggttatctccccatttctgcagtgcttgagaatggtcttcagatcttga

SEQ ID NO: 18 - DNA, 375 bp, synthetic construct
  misc_feature 1..375: note = Synthetic Polymer
  source 1..375: mol_type = other DNA
atggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaa

SEQ ID NO: 19 - DNA, 810 bp, synthetic construct
  misc_feature 1..810: note = Synthetic Polymer
  source 1..810: mol_type = other DNA
atgggtaaagagaaaacgcacgtcagtcgtccaagattgaactccaatatggatgcagacctgtacggttacaaatgggctagagataacgttggacaatctggtgcaactatatatagattgtatgggaagccagacgcaccagagttgtttctaaagcatgggaaaggctctgttgctaatgatgtgactgatgaaatggtacgtttgaattggctaacagagtttatgcccttgcctactattaagcattttattcgtactcccgatgacgcttggttgctaaccaccgcaattcctggtaaaactgcctttcaagttctggaagaatacccagattccggtgaaaacatcgttgacgccttggctgttttcctgcgaagacttcactctattcccgtatgtaattgtccctttaattcagacagagtttttagattggctcaggctcaatctaggatgaataatggtttggttgatgcaagtgacttcgatgacgaaagaaacggttggcctgtcgagcaggtgtggaaggaaatgcataagttacttccattttctcctgattctgttgtaacccacggtgatttttccctagacaaccttatattcgatgagggcaagttgattggttgtattgacgtcggcagagtgggtatcgccgataggtatcaagatttagcaatactgtggaattgtctaggagaattttcacccagtctgcaaaagagattgttccagaaatacggaattgacaaccccgatatgaataagttgcagtttcatttgatgttggacgagttcttctaa

SEQ ID NO: 20 - DNA, 1029 bp, synthetic construct
  misc_feature 1..1029: note = Synthetic Polymer
  source 1..1029: mol_type = other DNA
atgggaaagaaaccagagctgaccgcaacgagtgtcgaaaaatttcttattgaaaaatttgatagtgtgtccgatttaatgcagcttagtgaaggcgaagagtcacgtgctttctcattcgacgttggtggacgtggctacgttttgagagttaatagttgtgcagatggcttttataaggatcgttatgtataccgtcattttgctagtgcagccctgccaatcccagaggttttagatataggtgagtttagtgagtctcttacttattgtattagtcgtagagcccaaggtgttacccttcaggatttgccagagactgagcttcctgctgtattgcaacctgtcgctgaggctatggacgccattgccgcagcagatttatctcaaacgtcaggtttcggccccttcggcccacaaggcatcggacagtacacaacgtggcgtgactttatctgtgccatcgctgaccctcatgtctaccactggcaaacggtcatggatgacacggtgtccgcctctgtggcccaagcattggatgaactgatgctttgggctgaggattgtcccgaagtccgtcacctggttcacgctgacttcggctccaacaatgttttgaccgacaatggccgtatcaccgctgtcatcgactggtctgaggcaatgtttggcgactctcagtatgaagtcgccaatatatttttttggagaccctggttggcatgcatggaacagcaaactcgttactttgaaagacgtcatccagagttagctggtagtccacgtctgcgtgcttacatgttgcgtatcggcttagaccaactgtatcagtcacttgtcgatggtaactttgatgacgcagcatgggcacaaggacgttgtgacgctattgtacgttcaggtgcaggcacggtcggccgtacacaaattgcacgtagaagtgcagcagtctggaccgatggttgtgttgaggtccttgcagattcaggaaatagacgtccatctactcgtcctcgtgctaaggaataa

SEQ ID NO: 21 - DNA, 25 bp, synthetic construct
  misc_feature 1..25: note = Synthetic Polymer
  source 1..25: mol_type = other DNA
ggaattgtgagcggataacaattcc

SEQ ID NO: 22 - DNA, 1032 bp, synthetic construct
  misc_feature 1..1032: note = Synthetic Polymer
  source 1..1032: mol_type = other DNA
atggccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcgcatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaa

SEQ ID NO: 23 - DNA, 34 bp, synthetic construct
  misc_feature 1..34: note = Synthetic Polymer
  source 1..34: mol_type = other DNA
ataacttcgtataatgtatgctatacgaacggta

SEQ ID NO: 24 - DNA, 34 bp, synthetic construct
  misc_feature 1..34: note = Synthetic Polymer
  source 1..34: mol_type = other DNA
taccgttcgtataatgtatgctatacgaagttat

SEQ ID NO: 25 - DNA, 673 bp, synthetic construct
  misc_feature 1..673: note = Synthetic Polymer
  source 1..673: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcaca

SEQ ID NO: 26 - protein, 1106 aa, synthetic construct
  REGION 1..1106: note = Synthetic Polymer
MDREQGILPQDPFSNSVHVPKLRASSGGQPQKPVIQNSAPATARMLRNASSSTSAALLKELNTHEHSQRQHTPQKQPSLDAPAALVPVESATKQFHRTSIGDWEFSNTIGAGSMGKVKVAKHRVTHEVCAIKIVIRSAKIWQRNHQNDPEPETEEKRKKLRDEYKKELERDERTVREAALGKIMYHPNICRLFECYTMSNHYYMLFEIVQGVQLLDYIVSHGKLKETRVRQFARSIASALDYCHSNNIVHRDLKIENIMINNKGEIKLIDFGLSNMYDRRNLLKTFCGSLYFAAPELLSCRPYIGPEIDVWSFGVVLFVLVSGKVPFDDDSVPKLHAKIKRGKVEYPEFISPLCHSLLSQMLVVNPDHRVTLKAAMEHPWMTLGFAGPPSNYLPQRSPIVLPLDLSVVREIANLGLGNEEQIARDITNLISSREYEACVERWKLDQQKANIKGYSARDDSAIIAFHPLLSTYYLVDEMRKRKLAKGALKGQTSVLDTVKVSPDIPKTPAIPQKLETTDVEQPLLATVPPAYTSPHGQPAELEAMIEPAQPLSSAHPFEMDMTQQQHASRKTHIKHAPERQDRGGYNVHKNNSGGLNSLFRRLSGKRPHKNEAEWEPSSPPPQVHPFSVNDADRTSVRGVSPITQPAAVKNVTSNNSKNYLDPVDDSKLVRRVGSLRITNKEKQQVTSDFPRLPNFTIPEQPPKNAPIPIHAQPTTTGTTFQSNDHEIKKKLQASTSPNEQRGPPTLAPSQQRRLHPTARAKSLGHSRKQSLNFKFGGPANNQLPALPTKENYDVFEDAQITDNNLLNPEGKYSANTNVHIKPMTESQILFEAEHAPPGTMPSVEYPRTLFLKGFFSVQTTSSKPLPVIRYNIIAALCKLNIQFTEVNGGFVCVYRKTENLQIGDIRSPVIESRVTDDTDSDVANSSKLSSSSTANTRVNVIEDDSSSPSSARLKHRRKFSLGNGILNHIRKPTLDGTEFDDYDATVNTPVTPAPANVHSRSSSYHTESDNESMESLHDIRGGSDMILKNVPERNARQIDTVKEEETDDDDLGSINEGSTHRTPLKFEIHIVKVPLVGLYGVRFKKILGNAWIYKRLASKLLQELNL

SEQ ID NO: 27 - protein, 442 aa, synthetic construct
  REGION 1..442: note = Synthetic Polymer
MGKLAQLVLHPLELRAAIQFKFFKQSLHPRQPTNERETLKHCYELLALTSRSFCTVILELNPELRNAIMIFYLVLRALDTVEDDMTIKPDIKIPLLRSFDEKLNLKSWSFDGNSPDEKDRQVLVDFTDVLEEYHRLKPVYQDVIKDITHKMGNGMADYITDEEFNLNGVATVKDYDLYCHYVAGLVGEGLTHLIVEAGFGDPKLEDNMQLSESMGLFLQKTNIIRDYREDLDDGRSFWPKEIWSKYADSLSDFSKRENYEKGLDCISELVLNTMDHIKDVLVYLSSVYDFSSYNFCVIPQVMAIATLATVFRNEKVFETNVKIRKGTTCYLILKARTFEGACEIFSYYLRQIHHSCPITDANYIKIGIKCGELEQFLESLNPAPHVPPGATIPQTPHFVKAERKRKLDRELVPTLAIESLKCDVFLSLVALGFLGVIYSISS

SEQ ID NO: 28 - protein, 517 aa, synthetic construct
  REGION 1..517: note = Synthetic Polymer
MQFNWDIKTVASILSALTLAQASDQEAIAPEDSHVVKLTEATFESFITSNPHVLAEFFAPWCGHCKKLGPELVSAAEILKDNEQVKIAQIDCTEEKELCQGYEIKGYPTLKVFHGEVEVPSDYQGQRQSQSIVSYMLKQSLPPVSEINATKDLDDTIAEAKEPVIVQVLPEDASNLESNTTFYGVAGTLREKFTFVSTKSTDYAKKYTSDSTPAYLLVRPGEEPSVYSGEELDETHLVHWIDIESKPLFGDIDGSTFKSYAEANIPLAYYFYENEEQRAAAADIIKPFAKEQRGKINFVGLDAVKFGKHAKNLNMDEEKLPLFVIHDLVSNKKFGVPQDQELTNKDVTELIEKFIAGEAEPIVKSEPIPEIQEEKVFKLVGKAHDEVVFDESKDVLVKYYAPWCGHCKRMAPAYEELATLYANDEDASSKVVIAKLDHTLNDVDNVDIQGYPTLILYPAGDKSNPQLYDGSRDLESLAEFVKERGTHKVDALALRPVEEEKEAEEEAESEADAHDEL

SEQ ID NO: 29 - protein, 645 aa, synthetic construct
  REGION 1..645: note = Synthetic Polymer
MPAVGIDLGTTYSCVAHFANDRVEIIANDQGNRTTPSFVAFTDTERLIGDAAKNQAAMNPANTVFDAKRLIGRKFSDAETQADIKHFPFKVVDKGGKPNIQVEFKGETKVFTPEEISSMVLTKMKDTAEQFLGDKVNDAVVTVPAYFNDSQRQATKDAGLIAGLNVMRIINEPTAAAIAYGLDKKAEGEKNVLIFDLGGGTFDVSLLSIEDGIFEVKATAGDTHLGGEDFDNRLVNHFIAEFKRKNKKDLSSNQRALRRLRTACERAKRTLSSSAQTSIEIDSLFEGVDFYTSLTRARFEELCGDLFRSTIEPVEKVLKDAKLDKSQVNEIVLVGGSTRIPKVQKLVSDFFNGKEPNRSINPDEAVAYGAAVQAAILSGDTSSKTQDLLLLDVAPLSLGIETAGGIMTKLIPRNSTIPTKKSETFSTYADNQPGVLIQVYEGERAKTADNNLLGKFELSGIPPAPRGVPQIEVTFDMDANGILNVSAVEKGTGKAQQITITNDKGRLSKEDIEAMISEAEKYKDEDEKEAARIQARNALESYSFSLKNTLNEKEVGEKLDAADKESLTKAIDETTSWIDENQTATTEEFEAKQKELEGVANPIMTKFYQANGGAPGGAAPGGFPGAAGAGAEAPGADGPTVEEVD

SEQ ID NO: 30 - protein, 657 aa, synthetic construct
  REGION 1..657: note = Synthetic Polymer
MGKSIGIDLGTTYSCVAHFANDRVEIIANDQGNRTTPSFVAFTDTERLIGDAAKNQAAMNPANTVFDAKRLIGRKFDDPETQADIKHFPFKVINKGGKPNIQVEFKGETKVFSPEEISSMVLTKMKDTAEQYLGEKINDAVVTVPAYFNDSQRQATKDAGLIAGLNVQRIINEPTAAAIAYGLDKKDAGHGEHNILIFDLGGGTFDVSLLSIDEGIFEVKATAGDTHLGGEDFDNRLVNHFIAEFKRKTKKDLSTNQRSLRRLRTACERAKRTLSSSAQTSIEIDSLFEGIDFYTSITRARFEELCADLFRSTIEPVERVLKDSKLDKSQVHEIVLVGGSTRIPKVQKLVSDFFNGKEPNKSINPDEAVAYGAAVQAAILSGDTSSKTQDLLLLDVAPLSLGIETAGGIMTKLIPRNSTIPAKKSEIFSTYADNQPGVLIQVFEGERTRTKDNNLLGKFELSGIPPAPRGVPQIEVTFDMDANGILNVSAVEKGTGKTQKITITNDKGRLSKEDIERMVSEAEKFKDEDEKEAERVAAKNGLESYAYSLKNSAAESGFKDKVGEDDLAKLNKSVEETISWLDESQSASTDEYKDRQKELEEVANPIMSKFYGAAGGAPGGAPGGFPGGFPGGAGAAGGAPGGAAPGGDSGPTVEEVD

SEQ ID NO: 31 - protein, 613 aa, synthetic construct
  REGION 1..613: note = Synthetic Polymer
MADGVFQGAIGIDLGTTYSCVATYDSAVEIIANEQGNRVTPSFVAFTPEERLIGDAAKNQAALNPKNTVFDAKRLIGRAFDDESVQKDIKSWPFKVVNDNGNPLIEVEYLGETKQFSPQEISSMVLTKMKEVAEAKIGQKVEKAVVTVPAYFNDAQRQATKDAGAISGLNVLRIINEPTAAAIAYGLGAGKSEEEKHVLIFDLGGGTFDVSLLHIAGGVFTVKATAGDTHLGGQDFDTNLLEFFKKEFQKKTGKDISDDARALRRLRTACERAKRTLSSVAQTTVEVDSLFDGEDFTAEISRAKFEAINADLFKSTLEPVEQVLKDSKIEKSKVDDVVLVGGSTRIPKVQKLLSDFFDGKQLEKSINPDEAVAYGAAVQGAILTGQSTSEETKDLLLLDVIPLSLGVAMQGNVFAPVVPRNTTVPTIKRRTFTTVDDHQTTVQFPVYQGERVNCSENTLLGEFDLKNIPPMSAGEPVLEAIFEIDANGILKVTAVEKSTGRSANITISNSIGRLSSSEIEKMINDADKFKKADEDFANRHESKQKLEAYVSSIESTITDPILSSKLKRSAKDKIESALSDALAALELEDASGDDFRKAELALKRVVTKAMATR

SEQ ID NO: 32 - protein, 743 aa, synthetic construct
  REGION 1..743: note = Synthetic Polymer
MRDGEFFSFSLNSVARPMQSFFGKTNILANLRRNSETMSVPFGVDLGNNNTVIGVARNRGIDILVNEVSNRQTPSIVGFGAKSRAIGESGKTQQNSNLKNTVEHLVRILGLPADSPDYEIEKKFFTSPLIEKDNEILSEVNFQGKKTTFTPIQLVAMYLNKIKNTAIKETKGKFTDICLAVPVWFTEKQRSAASDACKVAGLNPVRIVNDITAAAVGYGVFKTDLPEDEPKKVAIVDIGHSTYSVLIAAFKKGELKVLGSASDKHFGGRDFDYAITKHFAEEFKSKYKIDITQNPKAWSRVYTAAERLKKVLSANTTAPFNVESVMNDVDVSSSLTREELEKLVQPLLDRAHIPVERALAMAGLKAEDVDTVEVVGGCTRVPTLKATLSEVFGKPLSFTLNQDEAIARGAAFICAMHSPTLRVRPFKFEDVNPYSVSYYWDKDPAAEDDDHLEVFPVGGSFPSTKVITLYRSQDFNIEARYTDKNALPAGTQEFIGRWSIKGVVVNEGEDTIQTKIKLRNDPSGFHIVESAYTVEKKTIQEPIEDPEADEDAEPQYRTVEKLVKKNDLEITGQTLHLPDELLNSYLETEAALEVQDKLVADTEERKNALEEYIYELRGKLEDQYKEFASEQEKTKLTAKLEKAEEWLYDEGYDSTKAKYIAKYEELASIGNVIRGRYLAKEEEKKQAIREKEESKKASAIAEKMAAERASREAAGSTNEQAQKNEENTKDADGDVSMNQDELD

SEQ ID NO: 33 - protein, 304 aa, synthetic construct
  REGION 1..304: note = Synthetic Polymer
MPVDSSHKTASPLPPRKRAKTEEEKEQRRVERILRNRRAAHASREKKRRHVEFLENHVVDLESALQESAKATNKLKQIQDIIVSRLEALGGTVSDLDLAVPEVDFPKFSDLELSTDLSSSTKSEKASTSTCRSSTEDLDEDGVAEYDDEEDEELPRKKNVLNDKSKNRTIKQEKLNELPSPLSSDFSDVDEEKSTLTHFQLQQQQQQQPVDNYVSTPLSLPEDSIDFINPGSLKIESDENFLLGSSTLQIKHENDTEYIPTAPSGSINDFFNSYDISESNRLHHPAAPFTANAFDLNDFVFFQE

SEQ ID NO: 34 - protein, 831 aa, synthetic construct
  REGION 1..831: note = Synthetic Polymer
MNGKHLLLQVLLVQLVAAVLDTQVGYIDWLVTSTGSFLDLSSCLFNYEQIYCLTEANDLIGLDSDAQITYRLHLDGPDQGKLTKLNNKKFGSVRGNYLDIFNEKGHLLHTEKFPSPIVDVYLDNSLLAVDLEGVVREIDLSTHSSKEVATLQSLACAMFSKVDDKVTIAFKGSNSDFVKIAILEDKVSTISTNISSVVHIKNNLLETDEGIYSIEGSTVKKILDGTAYLTDIGAISVDTVKNSVRSSGNSFEPQSKILKVHAEDEFIVVLTVDEVLEIDLETFDLSSVKENSLTEEYLNSVDYEIFFKNQEVQLIIQDRSARELIITNGVIQKVLDLSLNDVVDYSIVTLQPQLKAIEDEIIEEENSTFFKAYTSRLFNTLAALKENIKKREFTSLFQYDTSGQDQSFGLDKRLVIGCSHGKLSAYHLLTKTPQLSWEIQLPLIDEVSSFNEGEVSVLSGTTVFTIDAETGDILSETVATAEDPQKEFDIKSDDRTISGLKLINNEYSSTWTFKASPEEKILKVVRREDDNSNVASAGHILGNNSVLFKYLFQNLISAVLLNEHTNDIRFVILNAITGQQVYSDVHSGIDSNTNVNLIYDENFIVVSYFGSDPIPEQHIVVYDLYESLTPNKRVEPKDGLVSNFDTDTPIPQISSQSFLFPSRINFIAASRSKFGIASKWIISVLENGQIFAIPKVVLNSRRVVGRDLTSTEKQEYGMSVYSPFISLPENIFTISNIRNLVLDNNSNTLPSGKPILTVEPTGLASTSFVCLINSFNVYCTQISPSKKFDMLRENFDQYKLLLSIFGLLAIVLLVRPYVYSRNVQKLWTTKI

SEQ ID NO: 35 - protein, 677 aa, synthetic construct
  REGION 1..677: note = Synthetic Polymer
MLSLKPSWLTLAALLYAMLMVVVPFAKPVRADDVESYGTVIGIDLGTTYSCVGVMKSGRVEILANDQGNRITPSYVSFTEDERLVGDAAKNLAASNPKNTIFDIKRLIGMKFDSPEVQRDLKRLPYSVKSKNGQPIVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISDNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDFFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGAPPGQGFDDDDGDFDYDYDYDHDEL

SEQ ID NO: 36 - protein, 441 aa, synthetic construct
  REGION 1..441: note = Synthetic Polymer
MPIDIINTLVVKGTDGIPGWPIIKRYGLPFVALSLLKVYCGGKLNPWQRDVHGKVYILTGATAGVGSQLAEELAKGGAQLILLVKDPSSSWTVEFVDDLRERTGNPLVYAEQCDLADLHSVRKFATRWLDNTPPRRLDGIVGCAGEALPLGAARSTSSDGVERQVAVNYLGHFHLLALLSPSLRAQPADRDVRVVLTTCTTQAMGQVSLDDPLWLDSQYPSKRPWQVFGGAKLMLGCFAQEFQRRLDATPRGDKMPSKLRVNVVNPGFMRTASTARVLSFGSLWGLLLYLLLYPIWFILFKTPIQGAQSYLAALFAEHFIELPGGQFIQDCKIVKPARKELSDFTFQNKLYEKTEKLIDQLERQSAKQRVRSKPKSNSKSKPSKKSGTANVGPEKENDVFASALKATPPDLFPHQRADPAGNKYLDQLEKKLAEQSKKHST

SEQ ID NO: 37 - protein, 441 aa, synthetic construct
  REGION 1..441: note = Synthetic Polymer
MSFFSQLTGALDKPGFNWKLLIAGFSSAEFAFEAYLSYRQIKKLQEKGHQVPQSLKGKIEEDVALKSQDYSFTKLKFGIFSDAVNLLYNLTWIKFDILPKLWNLSGNLLANSLAFLPWKGTLVQSLVFVNLLSIAGLVVSLPLSYYSTFVIEEKFGFNKQTLKLWITDAIKGLLLSFVFGTAIYAGFLKIVDYFSDTFMFYMSVFMFVIQIFFIIFYPKFIQPLFNKLTPLEDGELKQSIEKLAADQKFPLDKLYVIDGSKRSSHSNAYFLGLPWGTKQIVIFDTLIEKSSVDEVTAVLGHEIGHWALSHTTKLLLINQVQLFSIFSLFALFFKNKSLYQSFGFSGQPVIIGFTLFSDVLKPFNAVLSFATNLLSRNYEYQADEYAVDLGYSSDLSSALISLHKENLSSLHVDWLYSAYSHSHPHLTERLQAIEFNAKKEK

SEQ ID NO: 38 - protein, 257 aa, synthetic construct
  REGION 1..257: note = Synthetic Polymer
MSREDSVYLAKLAEQAERYEEMVENMKTVASSGLELSVEERNLLSVAYKNVIGARRASWRIVSSIEQKEEAKGNQSQVSLIREYRSKIETELANICEDILSVLSEHLIPSARTGESKVFYFKMKGDYHRYLAEFAVGDKRKEAANLSLEAYKSASDVAVTELPPTHPIRLGLALNFSVFYYEILNSPDRACHLAKQAFDDAIAELETLSEESYKDSTLIMQLLRDNLTLWTSDMSETGQEESSNSQDKTEAAPKDEE

SEQ ID NO: 39 - protein, 527 aa, synthetic construct
  REGION 1..527: note = Synthetic Polymer
MRIVRSVAIAIACHCITALANPQIPFDGNYTEIIVPDTEVNIGQIVDINHEIKPKLVELVNTDFFKYYKLNLWKPCPFWNGDEGFCKYKDCSVDFITDWSQVPDIWQPDQLGKLGDNTVHKDKGQDENELSSNDYCALDKDDDEDLVYVNLIDNPERFTGYGGQQSESIWTAVYDENCFQPNEGSQLGQVEDLCLEKQIFYRLVSGLHSSISTHLTNEYLNLKNGAYEPNLKQFMIKVGYFTERIQNLHLNYVLVLKSLIKLQEYNVIDNLPLDDSLKAGLSGLISQGAQGINQSSDDYLFNEKVLFQNDQNDDLKNEFRDKFRNVTRLMDCVHCERCKLWGKLQTTGYGTALKILFDLKNPNDSINLKRVELVALVNTFHRLSKSVESIENFEKLYKIQPPTQDRASASSESLGLFDNEDEQNLLNSFSVDQAVISSKEAPEEIKSKPVGKAAYKQNSCPSLGSKSIKEAFHEELHAFIDAIGFILNSYRTLPKLLYTLFLVKSSELWDIFIGTQRHRDTTYRVDL